(19) United States
(12) Patent Application Publication
(10) Pub. No.: US 2023/0072511 A1
(43) Pub. Date: Mar. 9, 2023

(54) A SYSTEM TO ACHIEVE DIGITAL IMMORTALITY

(71) Applicant: ARIZONA BOARD OF REGENTS ON BEHALF OF THE UNIVERSITY OF ARIZONA, Tucson, AZ (US)

(72) Inventor: CHANGXU WU, Tucson, AZ (US)

(21) Appl. No.: 17/785,776

(22) PCT Filed: Dec. 19, 2020
(86) PCT No.: PCT/US20/66276

§ 371 Date: Jun. 15, 2022

Related U.S. Application Data
(60) Provisional application No. 62/951,366, filed on Dec. 20, 2019.

(51) Int. Cl. G06Q 50/26; G06F 16/28; G16H 10/20

(52) U.S. Cl. CPC G06Q 50/265; G06F 16/285; G16H 10/20

(57) ABSTRACT

The present disclosure provides a digital life system that includes personal information and memory collection logic to receive personal information and personal memory data from a first user, the personal information and memory collection logic to generate data tables based on the personal information and personal memory data; personal question and answer logic to generate responses to questions and statements submitted by a second user based on the data tables generated by the personal information and memory collection logic; and active talking logic to generate questions and statements directed towards the second user, without first receiving a question or statement from the second user, based on the data tables created by the personal information and memory collection logic.

### CROSS REFERENCE TO RELATED APPLICATIONS

This application is a U.S. National Stage Application of PCT/US20/66276 filed Dec. 19, 2020, and claims priority to U.S. Provisional Application 62/951,366 filed Dec. 20, 2019, both of which are incorporated herein by reference in their entirety.

### BACKGROUND

“When an old man dies, a library burns to the ground.”

To the individual human being, a digital copy of him/herself that can be preserved indefinitely not only can be a record of major events in his/her life, but also can be a record of thought process, dreams, personal preferences and other important information that cannot be recreated after a person dies. Such a digital copy can help friends and family better understand a person's life, life choices, thinking, preference, emotions, and judgements, etc. In a larger context, such knowledge accumulation of common people and their lives, beliefs, thinking, and preferences may offer significant historical evidence of given places and given times.

### BRIEF DESCRIPTION OF THE DRAWINGS

Features and advantages of various embodiments of the claimed subject matter will become apparent as the following Detailed Description proceeds, and upon reference to the Drawings, wherein like numerals designate like parts, and in which:

FIG. 1 depicts an illustrative functional block diagram for a digital life system, in accordance with at least one embodiment described herein;

FIG. 2 depicts an illustrative functional block diagram for the personal information and memory collection logic of the digital life system, in accordance with at least one embodiment described herein;

FIG. 3 depicts the process executed by experience logic of the digital life system in accordance with at least one embodiment described herein;

FIG. 4 depicts the process executed by personal question and answer preprocessing logic of the digital life system in accordance with at least one embodiment described herein;

FIG. 5 depicts the process executed by experience logic of the digital life system in accordance with at least one embodiment described herein;

FIG. 6A depicts the process executed by pronoun/keyword search logic of the digital life system in accordance with at least one embodiment described herein;

FIG. 6B further depicts the process executed by pronoun/keyword search logic of the digital life system in accordance with at least one embodiment described herein;

FIG. 6C depicts the process executed by interactive table search logic of the digital life system in accordance with at least one embodiment described herein;

FIG. 6D further depicts the process executed by interactive table search logic of the digital life system in accordance with at least one embodiment described herein;

FIG. 6E further depicts the process executed by interactive table search logic of the digital life system in accordance with at least one embodiment described herein;

FIG. 7A depicts the process executed by table specific logic of the digital life system in accordance with at least one embodiment described herein;

FIG. 7B further depicts the process executed by table specific logic of the digital life system in accordance with at least one embodiment described herein;

FIG. 7C further depicts the process executed by table specific logic of the digital life system in accordance with at least one embodiment described herein;

FIG. 7D further depicts the process executed by table specific logic of the digital life system in accordance with at least one embodiment described herein;

FIG. 7E further depicts the process executed by table specific logic of the digital life system in accordance with at least one embodiment described herein;

FIG. 7F further depicts the process executed by table specific logic of the digital life system in accordance with at least one embodiment described herein;

FIG. 7G depicts the process executed by consequence logic of the digital life system in accordance with at least one embodiment described herein;

FIG. 8 depicts the process executed by active talking preprocessing logic of the digital life system in accordance with at least one embodiment described herein;

FIG. 9 depicts the process executed by time management logic of the digital life system in accordance with at least one embodiment described herein;

FIG. 10A depicts the process executed by active dialogue logic of the digital life system in accordance with at least one embodiment described herein;

FIG. 10B further depicts the process executed by active dialogue logic of the digital life system in accordance with at least one embodiment described herein;

FIG. 10C further depicts the process executed by active dialogue logic of the digital life system in accordance with at least one embodiment described herein;

FIG. 10D further depicts the process executed by active dialogue logic of the digital life system in accordance with at least one embodiment described herein;

FIG. 11 depicts the process executed by knowledge update logic of the digital life system in accordance with at least one embodiment described herein;

FIG. 12 depicts an illustrative functional block diagram for the human self-awareness and consciousness logic of the digital life system, in accordance with at least one embodiment described herein;

FIG. 13 depicts an illustrative functional block diagram for a digital life system comprising a personal information collection system and a cloud-based storage and processing system, in accordance with at least one embodiment described herein;

Although the following Detailed Description will proceed with reference being made to illustrative embodiments, many alternatives, modifications and variations thereof will be apparent to those skilled in the art.

### DETAILED DESCRIPTION

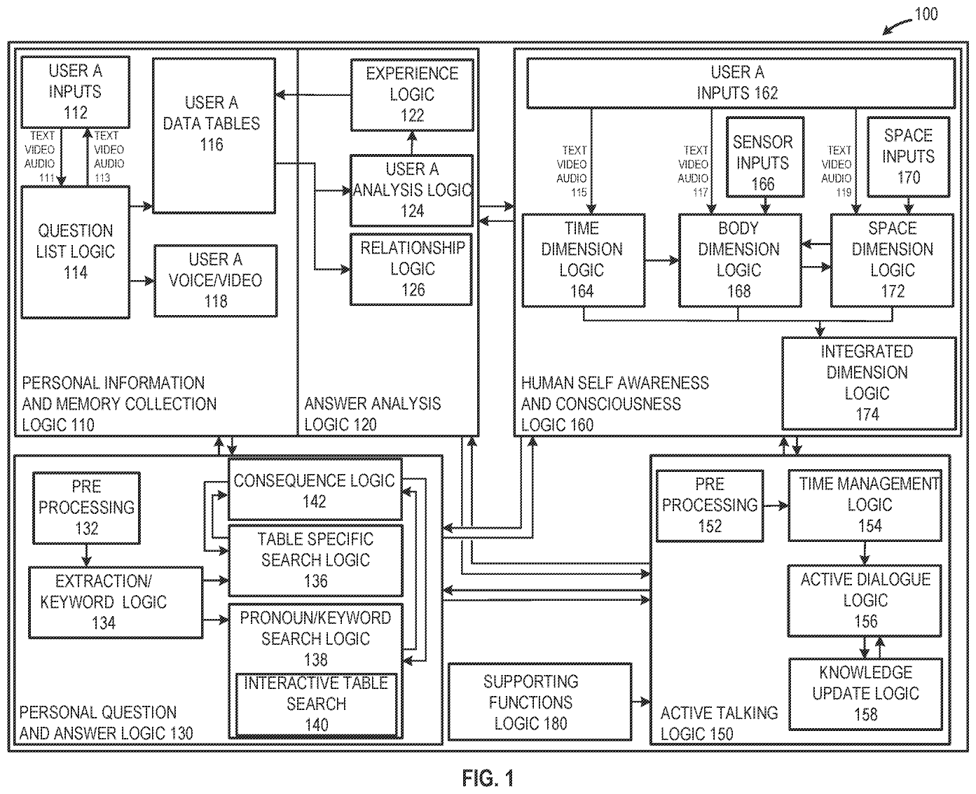

FIG. 1 illustrates an example functional block diagram of a digital life system 100. Consistent with several embodiments of the present disclosure, digital life system 100 collects information from a user, for example User A, and simulates a digital copy of the user. Digital life system 100 further allows other users, for example User B, to interact with the simulated digital copy. The digital copy is generated based on information related to a specific human user. Thus, digital life system is able to beneficially simulates a digital copy of a specific user, rather than simulating a general, non-specific person based on a large corpus of data. Further, digital life system is able to create a digital copy of a user that is able to interact with a human user based and generate verbal responses based on specific logic. Thus, digital life system 100 is beneficially able to create a digital copy to human user interaction without using a traditional AI approach, such as neural networks, deep learning, etc., which require training. According to the embodiment shown in FIG. 1, digital life system 100 includes personal information and memory collection logic 110, answer analysis logic 120, personal question and answer logic 130, active talking logic 150, human self-awareness and consciousness logic 160, and supporting functions logic 180.

### Personal Information and Memory Collection Logic

The personal information and memory collection logic 110 of FIG. 1 creates data tables based on information provided by a user, for example User A, in response to questions and/or prompts provided by question list logic 114. As explained in more detail below, question list logic 114 prompts the user with a series of questions related to various aspects of his or her life that they may not have otherwise considered or been able to remember. Thus, question list 114 logic may beneficially create a digital copy of the user that is able to consider and recall events, experiences, relationships, preferences, beliefs, and other personal information better than the human user. Further, personal information and memory collection logic 110 may store text, audio recordings, and video recordings related to the information provided by the user. According to the embodiment shown in FIG. 1, personal information and memory collection logic 110 includes User A inputs 112, question list logic 114, User A data tables 116, and user a voice/video 118. As User A, interacts with digital life system 100, question list logic 114 prompts User A to provide information. This information may be captured by digital life system 100 in the form of text, audio, and/or video and stored by user voice/video 118. Further, the information provided by User A may be related to, for example, User A's life experiences, relationships with others, favorites and/or personal preferences, dreams, regrets, hopes, attitudes, beliefs, opinions, summaries, personal information, needs, personality, valuable things, and other freestyle or unprompted information. The information provided by User A may be stored in User A data tables 116.

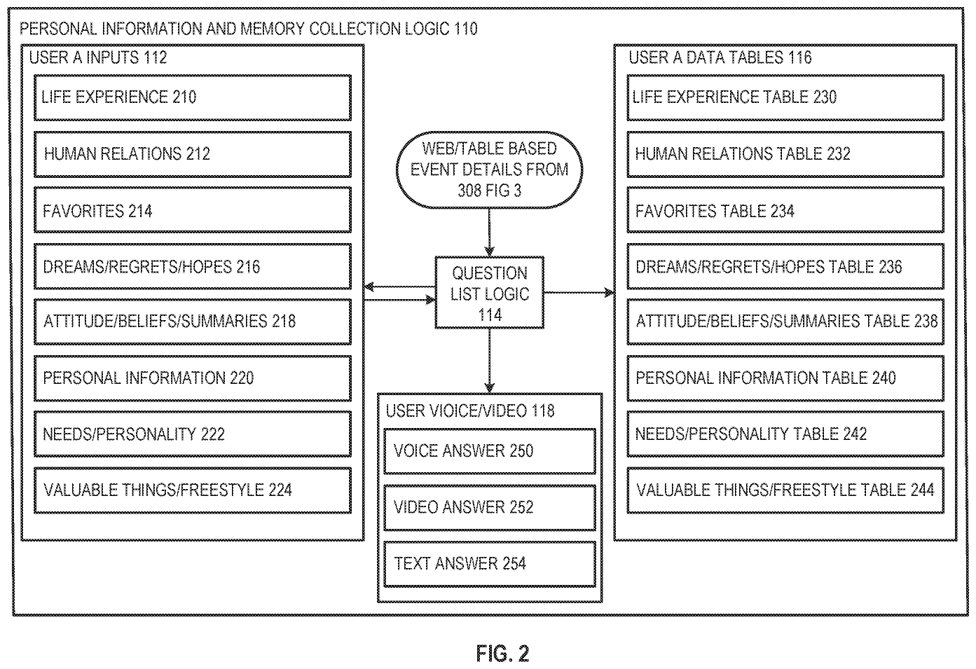

FIG. 2 illustrates a more detailed function block diagram of one embodiment of the personal information and memory collection logic 110 depicted in FIG. 1. For example, User A inputs 112 may include life experience 210, human relations 212, favorites 214, dreams/regrets/hopes 216, attitude/beliefs/summaries 218, personal information 220, needs/personality 222, and valuable things/freestyle 224. In one embodiment, question list logic 114 will prompt User A to answer questions related to each of the inputs comprised within User A inputs 112. Further, question list logic 114 may create User A data tables 116 based on information provided by User A. For example, according to the embodiment shown in FIG. 2, User A data tables 116 comprise life experience table 230, human relations table 232, favorites table 234, dreams/regrets/hopes table 236, attitude/beliefs/summaries table 238, personal information table 240, needs/personality table 242 and valuable things/freestyle table 244. In addition to creating User A data tables 116, question list logic 114 may cause the storage of information provided by the user in the form of audio, video, and text in user voice/video 118. For example, the User A's voice may be recorded while answering questions provided by question list logic 114 and may be stored in voice answer 250. Similarly, a video of User A may be recorded while answering questions provided by question list logic 114 and may be stored in video answer 252. Further, the text provided by User A while answering questions provided by question list logic 114 and may be stored in text answer 254. Question list logic 114 generates to capture a person's important life events, memory, beliefs, favorites, attitude etc. Thus, question list logic 114 may beneficially enable a more complete and efficient record of a person's experience compared to mining a person's daily dialogues with others or web publications (e.g., twitter, facebook, instagram) in which the key private information of that person (e.g., past life events, past memory, positive and negative attitudes, beliefs etc.) may not be usually disclosed completely or systematically. Further, digital life system 100 may offer a user flexibility in the timing needed to completed data collection component of the system. For example, users may answer the questions generated by question list logic 114 in 1-2 days or answer these questions in a daily basis as a diary. Of course, in other embodiments, in addition to the questionnaire provided by the present disclosure, other sources of information may be used, for example, data mining techniques from texts, Instagram, facebook, etc.

Moreover, the question-based data collection system of digital life system 100 beneficially allows User A discloses his/her important life experience, memory, believes, and private info in a voluntary manner with a respect of human privacy. It is not a “spyware” type of system that listens in on or mines a person's communications with others to generate information for a user.

According to one embodiment of the present invention, question list logic 114 will prompt User A to provide inputs related to life experience 210 by generating questions. Question list logic 114 will cause the inputs provided by User A to be organized and stored in life experience table 230. The questions and/or choices generated to prompt the user to provide life experience-related information may include, but are not limited, to the following: When did this event/experience happen? (which may also include a drop-down menu to allow the user to specify a date); How old were you when this event/experience happened? (which may also include a drop down menu to allow the user to specify an age); What happened?; Who were you with?; Where did it happen?; Why did it happen?; What are your feelings about this experience?; How did this experience affect your life?; What do you hope to tell others about this experience/event?; Please quantify the impact of this experience on your life as: −4 (Extremely Negative), −3 (Very Negative), −2 (Negative), −1 (A Little Negative), 0 (No Impact at All), +1 (A Little Positive), +2 (Positive), +3 (Very Positive), +4 (Extremely Positive) (which may also include a drop down menu to allow the user to make one of the foregoing choices). This list of questions may be generated for each event stored in life experience table 230. Further, after providing answers related to a first specific event, question list logic 114 may prompt the user to answer questions related to additional events by generating the same, or a similar, set of questions to the questions listed above. In addition to generating these questions, the question list logic 114 may provide the user with an option to select a photo from the user's phone album to be associated with a specific experience.

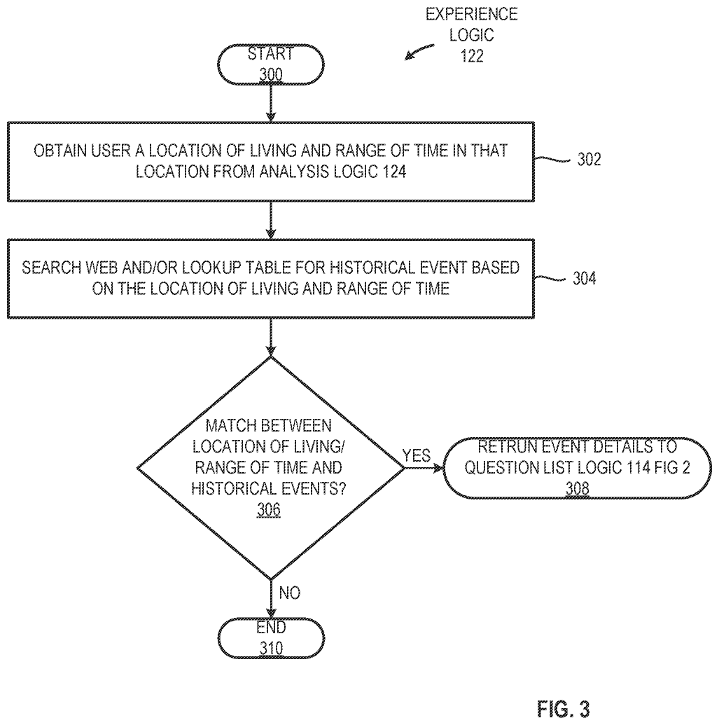

As discussed in more detail below, question list logic 114 may generate questions prompting User A to provide inputs related to life experience 210 based on experience logic 122. In one embodiment, experience logic 122 may determine historic events that occurred in the same or similar geographical area, compared to the User A's location of living during a specific time, based on personal information table 240 and/or analysis logic 124. Further, based on historical events determined by experience logic 122, questions list logic may provide User A with specific statements and/or questions related to that historical event. This is shown as an input to question list logic 114 in FIG. 2 as web/table based event details from 308 FIG. 3. For example, question list logic 114 may indicate to the user that, there was a historical event that occurred at User A's location of living during a specific time period. Further, question list logic 114 may provide the user with details about the historical event. After providing this information to the user, question list logic 114 may ask the user the follow questions about the historical event and store the user's answers in life experience table 230: What is your feeling about this experience?; How did this experience affect your life?; What do you hope to tell others about this experience/event?; Please quantify the impact of this experience on your life: −4 (Extremely Negative), −3 (Very Negative), −2 (Negative), −1 (A Little Negative), 0 (No Impact at All), +1 (A Little Positive), +2 (Positive), +3 (Very Positive), +4 (Extremely Positive) (which may also include a drop down menu to allow the user to make one of the foregoing choices).

According to one embodiment of the present invention, question list logic 114 will prompt User A to provide inputs related to human relations 212 by generating questions. Question list logic 114 will cause the inputs provided by the user to be organized and stored in human relations table 232. For example, question list logic 114 may prompt User A to define their relationships with other people by providing User A with a set of questions and/or choices for each of their family members, relatives, friends, etc. The questions and/or choices generated to prompt the user to provide human relations-related information may include, but are not limited, to the following: Name of this person; First Name; Middle Name; Last Name; User's relation with this person (user may be provided with a drop-down menu with choices such as father, mother, friend, aunt, uncle, classmate, etc.); When was this person born? (user may be provided with a drop-down menu allowing them to select a date); When and where did you meet this person?; When did this person pass away? (if applicable, user may be provided with a drop-down menu allowing them to select a date); If you know, where is/was this person located (now and/or in the past)?; What happened to this person?; What is/was this person's job (if applicable)?; What do you feel about this person?; Please quantify your feeling towards this person −4 (I extremely dislike/hate this person), −3 (I dislike/hate this person very much), −2 (I dislike/hate this person), −1 (I dislike/hate this person a little bit), 0 (Neutral), +1 (I like/love this person a little bit), +2 (I like/love this person), +3 (I like/love this person very much), +4 (I extremely like/love this person) (which may also include a drop down menu to allow the user to make one of the foregoing choices); Please quantify how much this person affects your life: 0 (No impact at all), 1 (Slight impact), 2 (Medium Impact), 3 (High impact), 4 (Highest Impact); If this is your last opportunity to talk with this person, what you hope to tell this person? This list of questions may be generated for each relation stored in human relations table 232. Further, after providing answers related to a first relationship, question list logic 114 may prompt the user to answer questions related to additional relationships by generating the same, or similar, sets of questions as the ones listed above. In addition to generating these questions, the question list logic 114 may provide the user with an option to select a photo from the user's phone album to be associated with the person and/or relationship.

According to one embodiment of the present invention, question list logic 114 will prompt User A to provide inputs related to favorites 214 by generating questions and/or categories of favorites for the user to select. Question list logic 114 will cause the inputs provided by the user to be organized and stored in favorites table 234. For example, question list logic 114 may prompt User A to define their favorite things by providing User A with a set of categories and/or questions related to favorites. The categories and/or questions generated to prompt the user to provide favorites-related information may include, but are not limited, to the following: color, brand, gift, lifestyle, diet, hobby, hope, plan, memory, age, dream, motto, fashion, joke, advice, refuser, drink, food, restaurant, coffee, candy, dish, fruit, dessert, pizza, ice-cream, beverage, alcoholic beverage, vegetable, salad, fish, meat, person, friend, relative, school subject(s), school teacher(s), classmate(s), coworker(s), boss(es), employee(s), holiday, season, day of the week, time of the day, date, celebrity, super hero, idols, star, movie, movie genre, TV program, TV channel, sport, sport game player, athlete, instrument, artist, art, artist, singer, music, song, actor, store, cartoon, game, fictional character, musician, entertainment, clothing, entertainment, game, transportation method, city or town, state, country, vacation, indoor or at home activities, hobby, outdoor activities, fun place, place of living, historical place/site, pet, animal, flower, plant, technology, book or story, car, phone brand, computer brand, toy, fashion brand, clothes brand, and perfume brand. Further, after providing responses related to the categories of favorites provided, question list logic 114 may prompt the user by asking if there is any favorites information the user hopes to record? In addition to generating these categories and/or questions, the question list logic 114 may provide the user with an option to select a photo from the user's phone album to be associated with any of the favorites selected.

According to one embodiment of the present invention, question list logic 114 will prompt the user to provide inputs related to dreams/regrets/hopes 216 by generating questions. Question list logic 114 will cause the inputs provided by the user to be organized and stored in dreams/regrets/hopes table 236. For example, question list logic 114 may prompt the user to define their dreams, regrets, and/or hopes by providing the user with a set of questions and/or choices. The questions and/or choices generated to prompt the user to provide dreams/regrets/hopes-related information may include, but are not limited, to the following: What is your dream, goal, or hope of your own life?; What is something you hope to do, but will never be able to do so, and why?; What is something you have done, but you regret what you did, and why?; What is your hope towards your parents or grandparents?; What is your hope towards your significant others, if you have any?; What is your hope towards your friends or relatives?; What is your hope towards your children or grandchildren, if you have any? In addition to generating these questions, the question list logic 114 may provide the user with an option to select a photo from the user's phone album to be associated with any of their answers.

According to one embodiment of the present invention, question list logic 114 will prompt the user to provide inputs related to attitude/beliefs/summaries 218 by generating questions. Question list logic 114 will cause the inputs provided by the user to be organized and stored in attitude/beliefs/summaries table 238. For example, question list logic 114 may prompt the user to define their attitude, either in general or about specific topics; their beliefs, either in general or about specific topics; or a summary of a life experience by providing the user with a set of questions and/or choices. The questions and/or choices generated to prompt the user to provide attitude/beliefs/summaries-related information may include, but are not limited, to the following: What is your belief for your life in general?; What is your attitude or belief towards society, money, work, study, marriage, love, family, poor people, rich people, regular people, needy people, children, spouse, parents, friends, animals, the world, aliens, environmental protection, technology?; What is your attitude or reaction towards a negative or unhappy event that happens to you?; What is your attitude or reaction towards a happy or good event that happens to you?; Any other attitude or belief you hope to record?; What are your personal interesting or valuable experience of your life, and what you have learned from your life? (summaries); What are your interesting or valuable experience(s) with your family members or relatives? And what you have learned from that? (summaries); What are your interesting or valuable experience(s) of your friends or neighbors or other social life? And what you have learned from that? (summaries); What are your interesting or valuable experience(s) of your love or marriage? And what you have learned from that? (summaries); What are your interests or valuable experience(s) of your study or work? And what you have learned from that? (summaries); What are your interesting or valuable experience(s) of your travel? And what you have learned from that? (summaries); What are your interesting or valuable experience(s) of using or generating technology? And what you have learned from that? (summaries); What are your interesting or valuable experience(s) of between you and animal? And what you have learned from that? (summaries); What is your social experience(s) and what have you learned from that?; What is your religion? Is most of your behavior following the expectations of your religion?; Any other interesting or valuable experience(s) or summary you hope to share or record? In addition to generating these questions, the question list logic 114 may provide the user with an option to select a photo from the user's phone album to be associated with each attituded/beliefs/summary-related answer.

According to one embodiment of the present invention, question list logic 114 will prompt the user to provide inputs related to personal information 220 by generating questions. Question list logic 114 will cause the inputs provided by the user to be organized and stored in personal information table 240. For example, question list logic 114 may prompt the user to provide biographical information by providing the user with a set of questions and/or choices. The questions and/or choices generated to prompt the user to provide personal information-related information may include, but are not limited, to the following: What is your full name (Last, middle, first name)?; What is your gender?; What is the date of your birth?; What day of the week were you born on? (the user may be provided with a drop-down menu from which to select a day of the week); Are there any special event(s) that are related to your birthday?; Where were you born? (City name and country); What is your current body height (the user may be provided with a drop down menu from which to select both feet and inches); What kind of health problem(s) do you have?; What is your life style?; Do you hope to record some secrets of yourself? (If yes, the user will be prompted to begin recording secretes); Are you an early bird or a night owl, or do you not follow a pattern?; Do you wear glasses or optical lenses?; Which school or university did you go to?; What were (are) your major(s) and when did (will) you graduate?; What is your nickname, job, salary, nationality, ethnicity, telephone, address, hair color, eye color?; What music instrument you can play?; Do you have any pets?; What kind of animal(s) is/are your pet(s) and what is/are their name(s)? Is there any other personal information you hope to record? In addition to generating these questions, the question list logic 114 may provide the user with an option to select a photo from the user's phone album to be associated with any personal information related question.

According to one embodiment of the present invention, question list logic 114 will prompt the user to provide inputs related to needs/personality 222 by generating questions and/or choices for the user to select from. Question list logic 114 will cause the inputs provided by the user to be organized and stored in needs/personality table 242. For example, question list logic 114 may prompt the user to rate the importunate of different aspects of their life and/or by providing the user with a set of questions related to their needs. The categories of needs that question list logic 114 prompts the user to rate may include, but are not limited to, the following: money, knowledge, health, beautifulness, cleanness, contribution to society, friendship, love, sex, fame and respect from others, child or children's happiness, personal family happiness, parental happiness, life goal realization. In addition to the categories above, question list logic 114 may prompt the user to select and rate one or more user-defined categories of needs. Further, for each of these categories, question list logic 114 may prompt the user to choose an importance rating between 0 and 10, wherein 0 means not important at all and 10 means the most important. In addition to providing categories to rank questions and/or choices generated to prompt the user to provide needs/personality-related information may include, but are not limited, to the following: Are you an introvert, neutral, or extrovert person?; Are you sometimes in a hurry or do you sometimes feel a sense of urgency?; Are you more interested in working with humans or more interested in working with machines, including computers?; Do you sometimes care more about yourself than others? Is there any other personal information you hope to record? In addition to generating these questions, the question list logic 114 may provide the user with an option to select a photo from the user's phone album to be associated with any needs/personality-related question.

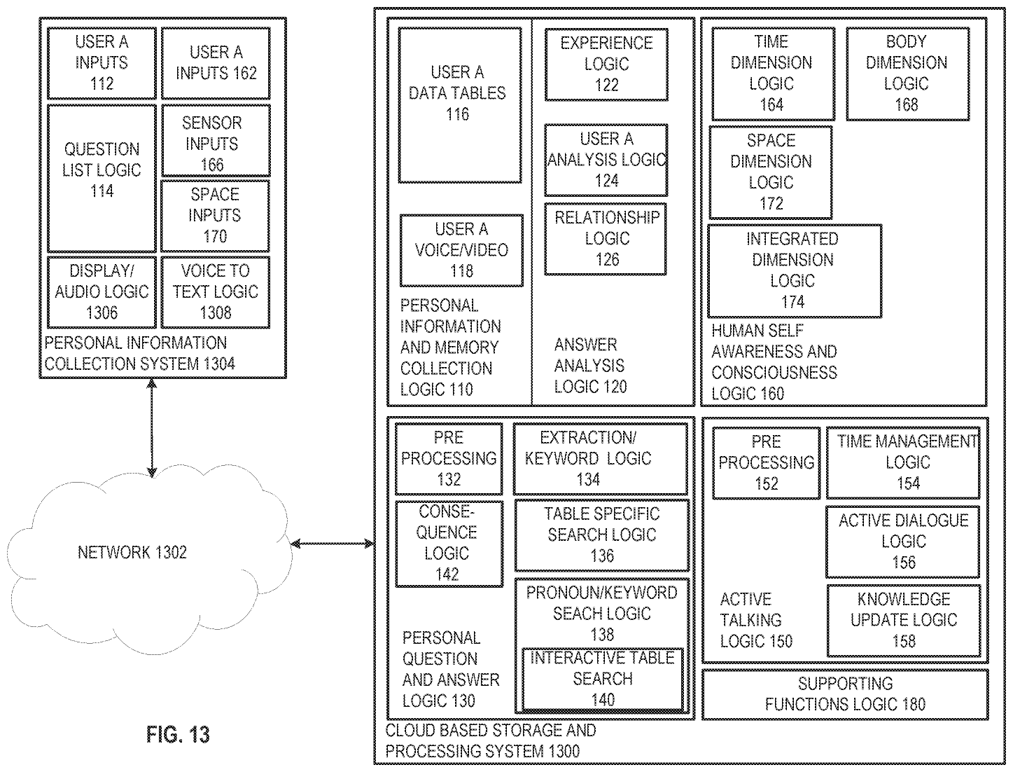

According to one embodiment of the present invention, question list logic 114 will prompt the user to provide inputs related to valuable things/freestyle 224 by generating questions and/or choices for the user to select from. Question list logic 114 will cause the inputs provided by the user to be organized and stored in valuable things/freestyle table 244. For example, question list logic 114 may prompt the user to record any additional information that they think would be valuable and/or useful in the form of text, photographs, audio, video, etc. In one embodiment of the present invention, when the user provides any information related to User A inputs 112, question list logic 114 will cause the user's voice to be recorded and stored in voice answer 250 comprised in user a voice/video 118. In another embodiment, when the user provides any information related to User A inputs 112, question list logic 114 will cause the recognition of the users voice, cause the users voice to be converted to text, and store the text in text answer 254 comprised in user voice/video 118. FIG. 13 shows one example embodiment where user a voice/video 118 is comprised within cloud-based storage and processing system 1300.

Returning to FIG. 1, the exemplary embodiment of the digital life system 100 is shown as further comprising answer and analysis logic 120. The answer and analysis logic 120 of FIG. 1 performs operations related to analyzing the information comprised in the User A data tables 116. Specifically, answer and analysis logic 120 comprises User A analysis logic 124, relationship logic 126, and experience logic 122.

User A analysis logic 124 may perform operations based on information stored in needs/personality table 242 to determine a degree of selfishness of a user. These operations may be performed by personality analysis logic comprised within User A analysis logic 124. In one embodiment, the personality analysis logic comprised within User A analysis logic 124 determines a degree of selfishness of the user based on the answer the user provided to the question “Do you sometimes care more about yourself than others?” as an input for need/personality 222. Based on the answer stored in needs/personality table 242, the personality analysis logic comprised within User A analysis logic 124 will determine degree of selfishness score between 0-5, wherein 0 equates to an answer similar to “No, I always/usually/etc. care more about others,” wherein 1 equates to an answer similar to “No, I do care others,” wherein 2 equates to an answer similar to “No,” wherein 3 equates to an answer similar to “Yes, I sometimes care more about myself than others,” wherein 4 equates to an answer similar to “I always care more about myself than others,” and wherein 5 equates to an answer similar to “I only care about myself.”

Additionally, User A analysis logic 124 may perform operations based on information stored in personal information table 240 to determine a major location in which the user spent his or her childhood. User A analysis logic may further perform operations based on personal information table 240 to determine a major location in which the user spent his or her life. These operations may be performed by geographic and population analysis logic comprised within User A analysis logic 124. In one embodiment, the geographic and population analysis logic comprised within User A analysis logic 124 utilizes pre-prepared look-up table to determine the typical favorite food, and popular religions and beliefs related to the user's major childhood living location and the user's major lifetime living location.

Relationship logic 126 may perform operations based on information stored in human relations table 232. In one embodiment, the relationship logic 126 determines a direction and degree of relationship between the user (for example, User A) and another user (for example, User B). For example, relationship logic 126 may determine a direction and degree of relationship between User A and User B based on the information stored in User A's human relations table 232 related to a question asking User A to quantify his or her feelings towards User B. In another embodiment, relationship logic 126 may determine the degree of closeness and understanding between the user (for example, User A) and another user (for example, User B). For example, relationship logic 126 may determine the degree of closeness and understanding between User A and User B based on a relation word (e.g. father, aunt) stored User A's human relations table 232 related to the question asking User A to identify his or her relationship with User B.

Experience logic 122 may perform operations based on based on the major location in which the user spent his or her childhood and/or the user's major location of living determined by User A analysis logic 124. In one embodiment, experience logic 122 obtains the major location in which the user spent his or her childhood and/or the user's major location of living, searches the web and/or a lookup table for historical events that occurred in the same or similar location during the same or similar time period. If there is a match between the major location in which the user spent his or her childhood and/or the user's major location of living and the historical event, experience logic 122 will provide details related to the historical event to question list logic 114. Based on the event details, question list logic 114 prompts the user to answer questions related to the event. In another embodiment, experience logic 122 may perform operations based on various locations of living of the user other than the major location of living or the major childhood location of living. Thus, experience logic 122 may beneficially identify events from a user's life that they would otherwise not be able to remember.

FIG. 3. shows one embodiment of experience logic 122. In this example, the operations performed by experience logic 122 begin at operation 300. Next, at operation 302, experience logic 122 obtains a user's location of living determined by User A analysis logic 124. In another embodiment, experience logic 122 may obtain a user's location of living determined by geographic and population analysis logic comprised in User A analysis logic 124. At operation 302, experience logic 122 may search the web, a lookup table, or another source other than User A data tables 116 for historical events that occurred at location proximate to the user's location of living and during a time period during which the user lived in that location. A location may be identified as proximate to the user's location of living, for example, by a specified search radius. In some embodiments, the search radius may be specified as being within 10 miles, 25 miles, 50 miles, 100 miles, etc. of the user's address. In other embodiments, a location may be identified as proximate to the user's location of living based on whether the name of the city, town, state, and/or country in which the event occurred matches the name of the city, town, state, and/or country of the user location of living. At operation 306, experience logic 122 determines whether there is a match between the user's location of living and any historical events. If there is a match, experience logic 122 will return details related to the historical even to question list logic 114 at operation 308. Then, question list logic 114 prompts the user to answer specific questions related to the identified even. For example, question list logic 114 may ask the user if they remember this event. If the user remembers the event, question list logic 114 may prompt the user to share any experiences the user remembers related to the event. Experience logic 122 may identify multiple events, and thus return multiple events to question list logic 114. If there are no historical events identified that match the user's location of living, the operations of experience logic 122 end at operation 310.

### Personal Question and Answer Logic

Returning to FIG. 1, the exemplary embodiment of the digital life system 100 is shown as comprising personal question and answer logic 130. As a second user (for example, User B) interacts with digital life system 100, the personal question and answer logic 130 of FIG. 1 performs operations related to responding to questions and/or statements from the second user (for example, User B) based, in part, on information collected about a first user (for example, User A). Specifically, personal question and answer logic 130 comprises pre processing 132, extraction/keyword logic 130, table specific search logic 136, pronoun/keyword search logic 138, interactive table search 140, and consequence logic 142.

### Preprocessing

Preprocessing 132 embodied in FIG. 1 generally performs operations related to identifying User B, determining the relationship between User A and User B, determining any table-specific activation terms were included in a statements and/or questions provided by User B, and greeting User B.

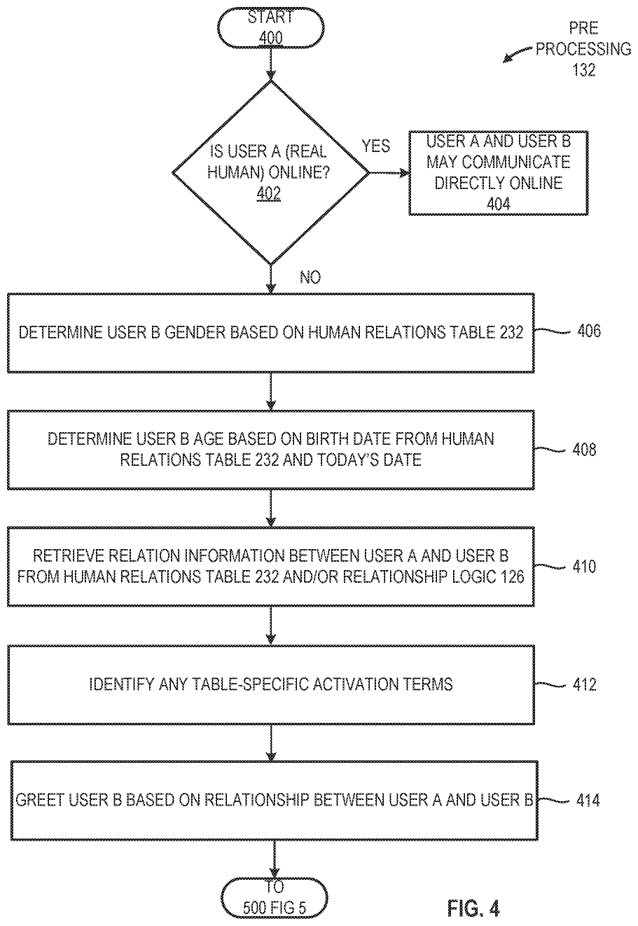

FIG. 4 shows one embodiment of preprocessing 132. In this example, the operations performed by preprocessing 132 begin at operation 400. Next, at operation 402, preprocessing 132 determines if both User A and User B are online. For example, User A may be a real human who can directly talk to User B rather than a digital copy of User A. If User A and User B are online, the process continues at operation 404 and User A and User B may communicate directly online. If User A is not online then the process continues to operation 406. Operation 402 beneficially allows User B to communicate with either User A (a real human), if User A is available, or with a digital copy of User A, if User A is unavailable. Next, at operation 406, preprocessing 132 determines the gender of User B based on human relations table 232. For example, information provided by User A as part of human relations 212 may comprise information about User B, include User B's gender, which is stored in human relations table 242. If human relations table 232 does not explicitly contain User B's gender, then preprocessing 132 may determine User B's gender based on User B's relation to User A as specified in human relations table 232. For example, if User B is User A's uncle, then preprocessing 132 may determine that User B is a male. If User B's relation to User A is not gender-specific, then preprocessing 132 may search other entries related to User B in human relations table 232 for gender-specific pronouns or other indications of User B's gender. For example, User B may be referred to as “he” in an entry under human relations table 232. In that case, preprocessing 132 will determine that User B is a male.

At operation 408, preprocessing 132 determines the age of User B based on human relations table 232 and the current date. For example, information provided by User A as part of human relations 212 may comprise User B's date of birth, which is stored in human relations table 232. Preprocessing 132 may calculate User B's age based on the time different between the current data and User B's date of birth. At operation 410, preprocessing 132 retrieves relation information between User A and User B. For example, information provided by User A as part of human relations 212 may comprise User A's relation to User B's (which is stored in human relations table 232).

At operation 410, preprocessing 132 retrieves the relationship information between User A and User B based on human relations table 242 and/or relationship logic 126. For example, based on the statements and/or questions received by digital life system 100, and based on which user is speaking (for example, User B) and to whom the user is speaking (for example, User A), preprocessing 132 will search human relations table 242 to retrieve corresponding information of the relationship (for example, the relation information between User A and User B).

At operation 412, preprocessing 132 identifies any table specific activation terms that are included in User B's statement and/or question. In one embodiment, each table within User A data tables 116 will be associated with a list of activation terms. Further, preprocessing 132 may contain logic to identify synonyms the table-specific activation terms comprised in User B's statement or question.

For example, life experience table 230 may be associated with the following table-specific activation terms: a string of words containing “you” and “live/visit/stay/travel/go to”; a string of words containing “you” and “lived/visited/stayed/traveled/went to”; “did you live/visit/stay/travel/go to”; “have you been/lived/stayed/traveled/went to”; a string of words containing “when/where” and only “you” as the pronoun in the statement and no other pronouns and no words related to the general name for family member and/or relative member (e.g., aunt), or any person's name within human relations table 232; “when you are/were a child/kid/in school” or “your childhood” (for childhood-related terms, age 0-18); “when you are/were a teenage/in middle school/in high school” (for teenage related terms, age 12-18); “you were/are young” (for terms related to age 0-60); “you were/are in middle age (for terms related to age 40-60); “you were/are old”(for terms related to ages over 60).

Human relations table 232 may be associated with the following table-specific activation terms: any word related to the general name for family member and/or relative member (e.g., aunt); any person's name within human relations table 232; a string of words containing “like/love/dislike/do not like/don't like/hate” and “you” and “me/I”; a string of words containing “you” and “like/love/dislike/hate” and “he/him/she/her/they/them/someone.”

Favorites table 234 may be associated with the following table-specific activation terms: a string of words containing “like/love” but not “You/I” appearing together in the statement, or any word related to the general name for family member and/or relative member (e.g., aunt), or any person's name within human relations table 232; a string of words containing “dislike/don't like/do not like/hate” but not You/I″ appearing together in the statement, or any word related to the general name for family member and/or relative member (e.g., aunt), or any person's name within human relations table 232; a string of words containing “favor/favorites/favorable/favor” but not You/I″ appearing together in the statement, or any word related to the general name for family member and/or relative member (e.g., aunt), or any person's name within human relations table 232.

Dreams/regrets/hopes table 236 may be associated with the following table-specific activation terms: a string of words containing “you/your” and “regret/regrets/regretting/repent/regretted/guilt/guilty/shame/repentance”; a string of words containing “you/your” and “dream/life goal/hope/expectation.”

Attitude/beliefs/summaries table 238 may be associated with the following table-specific activation terms: “your attitude/belief/thinking/reaction/thoughts/understanding/experience/summary/summaries/etc.”; “What you think/believe . . . ”; “What you learned from . . . ”; “What you have learned from . . . ”; “What can you learn from . . . ”; “What do you think about . . . ”; “Can/would you (please) tell/inform me/us (more) about your. . . . ”; “Can/would you (please) share with me/us (more) . . . ”; a string of words not containing “money/cash/property”; “Your summary/summaries of . . . ”; a string of words only containing “you or your” used as pronouns and “experience/experienced”; a string of words only containing “you or you,” with no other pronouns and no words related to the general name for family member and/or relative member (e.g., aunt), or any person's name within human relations table 232, and with the words “attitude/belief/thinking/reaction/thoughts/understanding etc.”; “religion/ religious/Christianity/Islam/Nonreligious/Hinduism/Buddhism/Heaven/Hell/ Confucianism/Jainism/Judaism/Shinto/Sikhism/Taoism/Zoroastrianism.”

Personal information table 240 may be associated with the following table-specific activation terms: a string of words containing only “you or you,” with no other pronouns and no words related to the general name for family member and/or relative member (e.g., aunt), or any person's name within human relations table 232, and with the words “birthday/eye color/hair color/body/weight/job/salary/country/nationality/ethnicity/live/belief/gender/ telephone/education/health/medical/conditions/body type/life/style/birthmarks/body features.”

Needs/personality table 242 may be associated with the following table-specific activation terms: “can you give me cash/money”; “may I get cash/money from you.” In addition to the exemplary list of table-specific activation terms provided above, preprocessing logic may also utilize a list wherein, given a specific statement with some general terms, a specific table is activated.

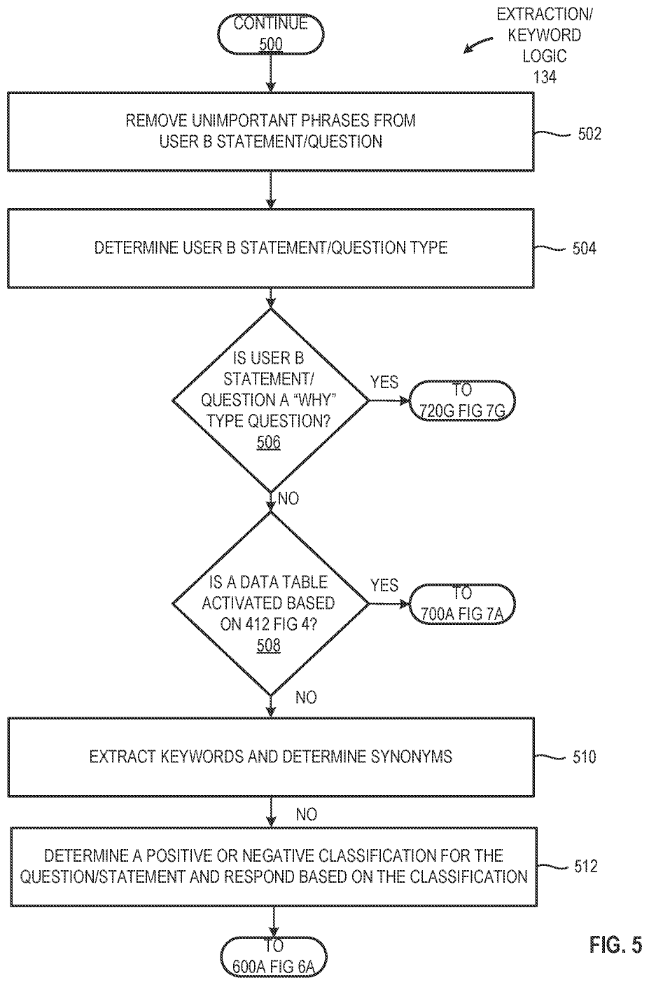

At operation 414, preprocessing 132 causes digital life system 100 to greet User B. The greeting provided to User B may be based on the relationship determined between User A and User B. For example, if User B is at a higher relationship level than User A, digital life system 100 may provide a greeting to User B such as “Hello, [relationship word]” wherein the relationship word is User B′ relation to User A. For example, the relationship word may be “mom/father/mother/grandpa/etc.” User B may be at a higher relationship level when User B's relation to User A is generally a more senior relationship. For example, if User B is the mother of User A, then User B is more senior, and thus at a higher relationship level compared to User A. If User B is at a lower or the same relationship level compared to User A, digital life system 100 may provide a greeting to User B such as “Hello, [User B's first name].” User B may be at a lower or the same relationship level compared to User A when User B's relation to User A is generally a less or equally senior relationship. For example, if User B is the child of User A, then User B is less senior, and thus at a lower relationship level compared to User A. Similarly, if User B is the friend of User A, then User B is equally senior, and thus at the same relationship level compared to User A. Other relations where User B would be at the same or lower relationship level compared to User A may be, for example, brother, sister, friend, daughter, son, etc. Upon the completion of operation 414, preprocessing 132 operations end and extraction/keyword logic 134 operations begin at FIG. 5 operation 500.

### Extraction/Keyword Logic

Extraction/keyword logic 134 embodied in FIG. 1 generally performs operations related determining and extracting terms and keywords from statements and/or questions provided by User B to facilitate the provision of responses to User B based, in part, on in the information comprised in User A data tables 114. More specifically, extraction/keyword logic 134 may operate by removing unimportant phrases from the statements and/or questions provided by User B, determining the type of statement and/or question provided by User B, determining whether the statement and/or question provided by User B is a “why” type question, determining whether a data table comprised in User A data tables 116 is activated based activation-specific terms identified by preprocessing 132, and extracting keywords and determining synonymous of keywords comprised in User B's statement and/or question, and determining a positive or negative classification for User B's question and/or statement. Thus, extraction/keyword logic 134 may beneficially enable to digital life system 100 to analyze statements provided by User B using keywords and grammar classifications, and based on that analysis, cause digital life system 100 to provide a response from a digital copy of User A that considers and recalls events, experiences, relationships, preferences, beliefs, and other personal information better than a human user.

FIG. 5 shows one embodiment of extraction/keyword logic 134. In this example, the operations performed by extraction/keyword logic 134 begin at operation 500. Next, at operation 502, extraction/keyword logic 134 removes unimportant phrases and/or words from the statement and/or questions provided by User B. For example, during operation 502, extraction/keyword logic 134 may remove the following terms from User B's statement and/or question: “Can/Could/Will/Would you [please] tell/inform me . . . ”; “I [just] hope/want to know . . . ”; “I am wondering”; “[Please] Inform/tell me . . . ”; “May I ask a question . . . ”; “I hope to ask a question . . . ”; “Here is my question . . . ”; “My question is . . . ”; “I guess/guessed/think/thought/am wondering/am thinking/”; etc.

At operation 504, extraction/keyword logic 134 determines the type of statement and/or question provided by User B. Extraction/keyword logic 134 may determine the type of statement and/or question based on a pre-prepared question type table. This pre-prepared question type table may store different types of grammatical structures that related to different types of statements and/or questions. For example, a phrase beginning with the words “Do you . . . ?” may related to a specific question type. To perform operation 504, extraction/keyword logic 134 may first determine whether the type of statements and/or question provided by User B is question. Next, if the statements and/or question is determined to be a question, extraction/keyword logic 134 may determine the type of question. For example, question types may be classified as: Yes/No questions, “why” questions (cause questions), “what” questions, “who” questions, “where” questions, “how many/much” questions, etc. Finally, extraction/keyword logic 134 may determine whether there are multiple question types contained in User B's question and/or statement.

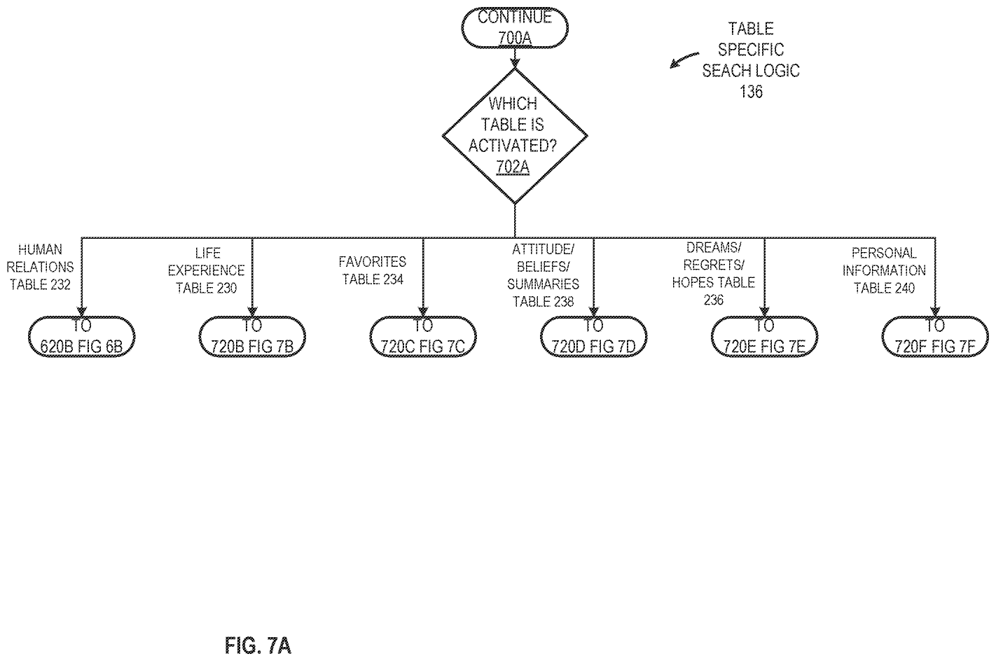

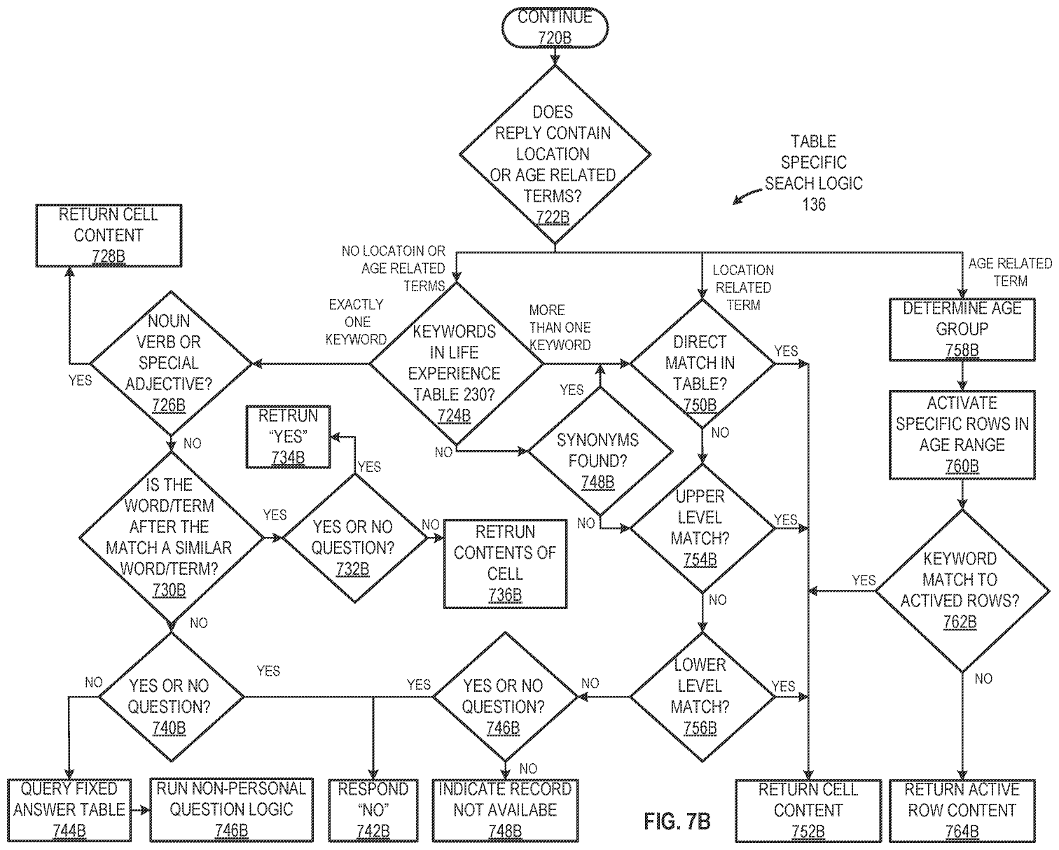

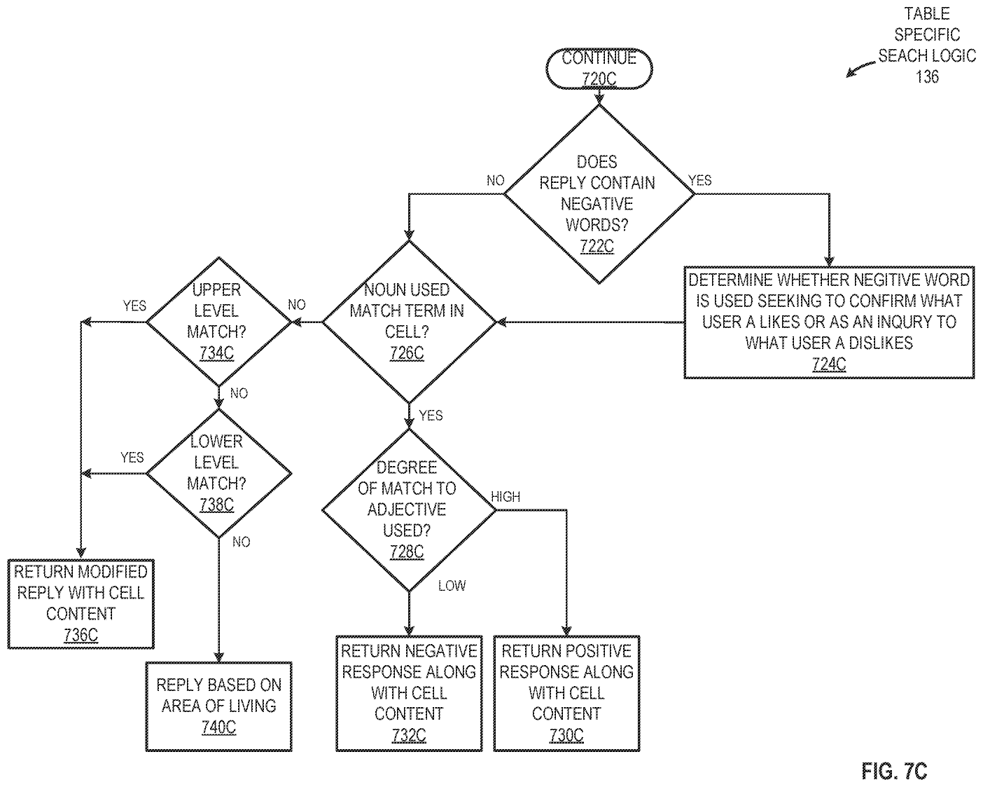

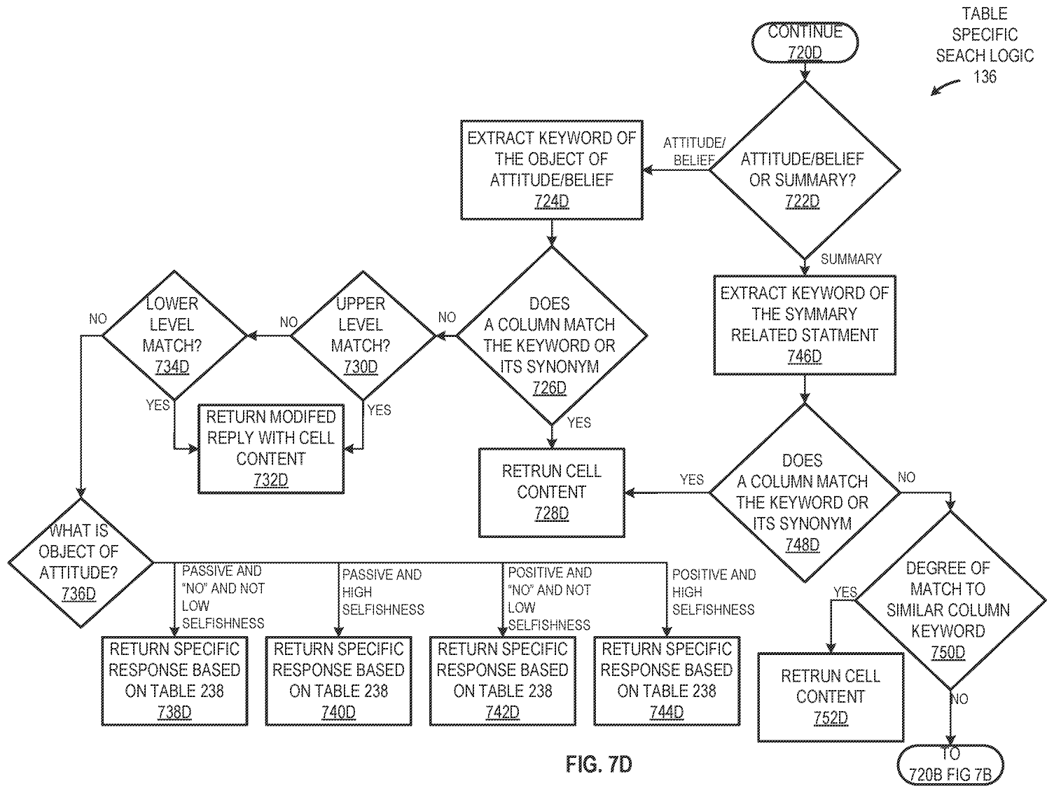

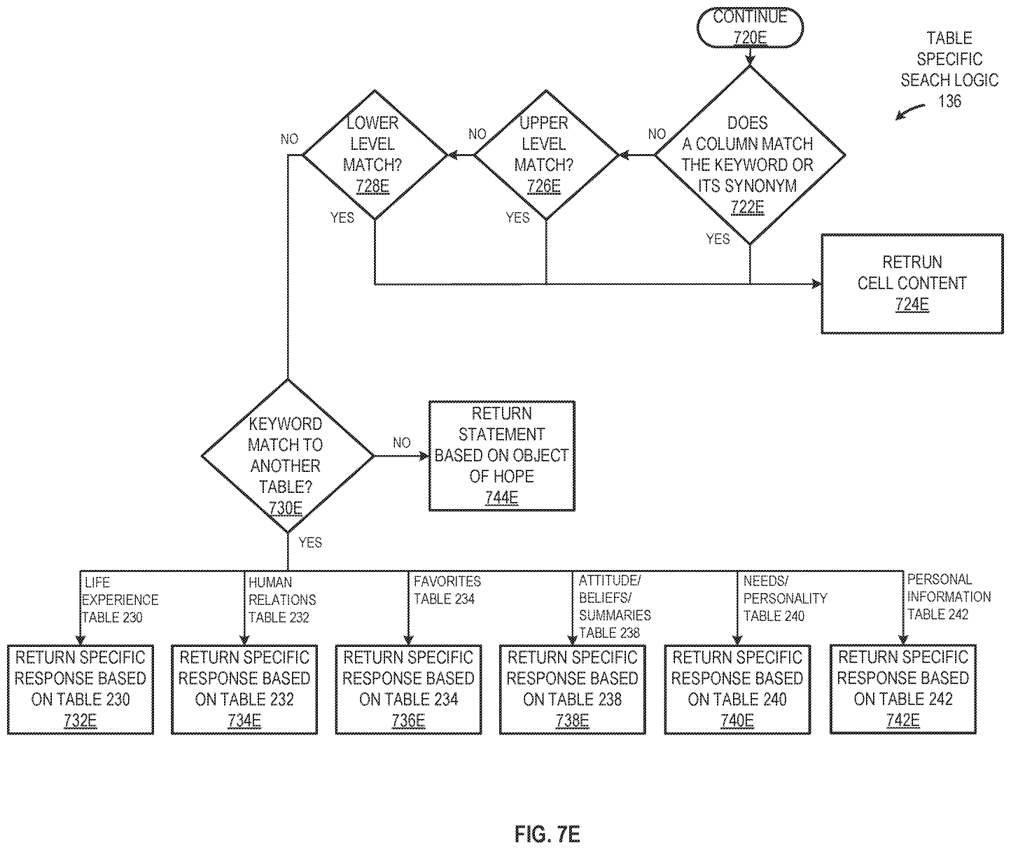

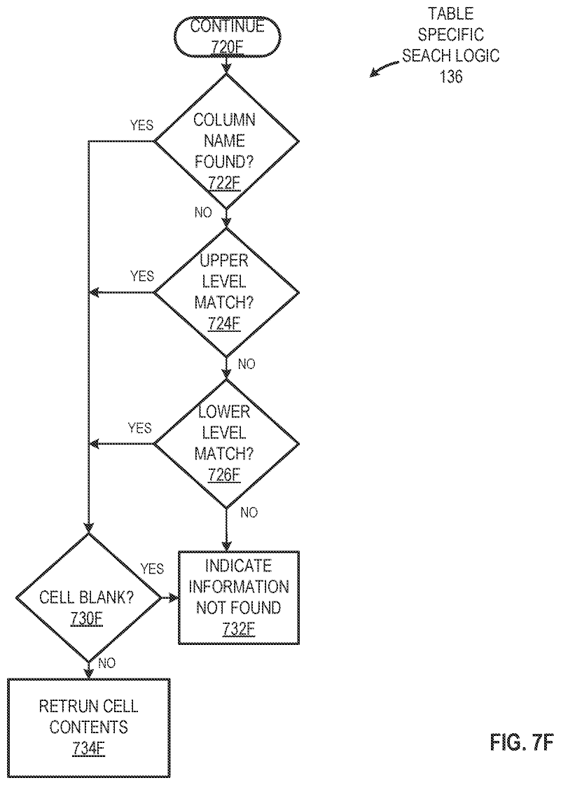

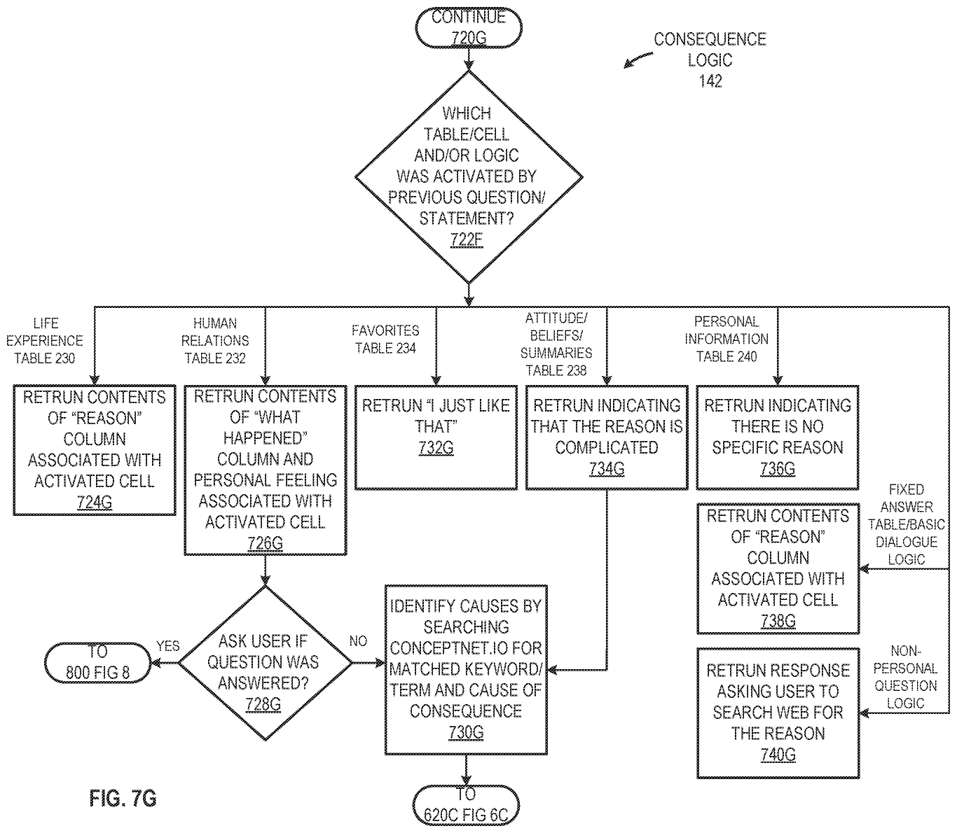

At operation 506, extraction/keyword logic 134 determines whether the process should continue to consequence logic 142. If a “why” type question was identified at operation 504, then extraction/keyword logic 134 will activate consequence logic 142 and the process continues to operation 720G of FIG. 7G. If a “why” type question was not identified, the process to continues to operation 508. “Why” type questions are explained in more detail under the Consequence Logic heading below.

At operation 508, extraction/keyword logic 134 determines whether the process should continue to table specific search logic 136 based on table-specific activation terms identified by preprocessing 132 at operation 412 of FIG. 4. If table-specific activation terms were identified by preprocessing 132, then extraction/keyword logic 134 will activate table specific search logic 136 and the process continues to operation 700A of FIG. 7A. If no table-specific activation terms were identified by preprocessing 132, then the process continues to operation 510.

At operation 510, extraction/keyword logic 134 extracts keywords and/or terms from User B's question and/or statement and determines synonyms of those keywords and/or terms. For example, first, extraction/keyword logic 134 may extract keywords and/or terms such as nouns, verbs, negative words (e.g not, doesn't, don't, didn't), adjectives, terms, and/or word combinations (e.g., country road). Then, according the keyword and/or term's usage, extraction/keyword logic 134 may determine whether the word is used as a verb, noun, adjective, etc. (because some words may use as verb, or noun, or adjective etc. depending on its context). Finally, extraction/keyword logic 134 may determine synonyms of the identified keywords and/or terms. In one non-limiting embodiment, extraction/keyword logic 134 may identify 2-3 synonyms for each keyword and/or term.

At operation 512, extraction/keyword logic 134 determines a positive or negative classification for User B's question and/or statement. In one non-limiting example, extraction/keyword logic 134 may determine a positive or negative classification based on the following criteria using a pre-prepared database. For example, if the statement contains a string of words comprising “you” or “your” with no other pronouns, no negative words (e.g. no, not, dis-, un- etc.), and a negative adjective (e.g. stupid) or a negative noun (e.g., fool), then it will be classified as a negative statement. Similarly, if the statement contains a string of words comprising “you” or “your” with no other pronouns, negative words (e.g. no, not, dis-, un- etc.), and a positive adjective (e.g., smart) or a positive noun (e.g., wisdom), then it will be classified in to negative statement. If extraction/keyword logic 134 classifies User B's statement as a negative statement, as in the two preceding examples, then it will cause digital life system to return a response to User B. In one example, the response to a negative statement may be “sorry, I do not accept that since everyone has something good.” Further, if extraction/keyword logic 134 classifies multiple, separate statements from User B as negative, the it may cause digital life system 100 to return a response indicating that the digital copy of User A no longer wishes to communicate with User B. In one non-limiting example, if more than 3 statements from User B are classified as negative, digital life system may return a response saying “Sorry, I don't hope to talk with you any longer. You don't respect others.” This may cause the system to temporarily end communication with User B.

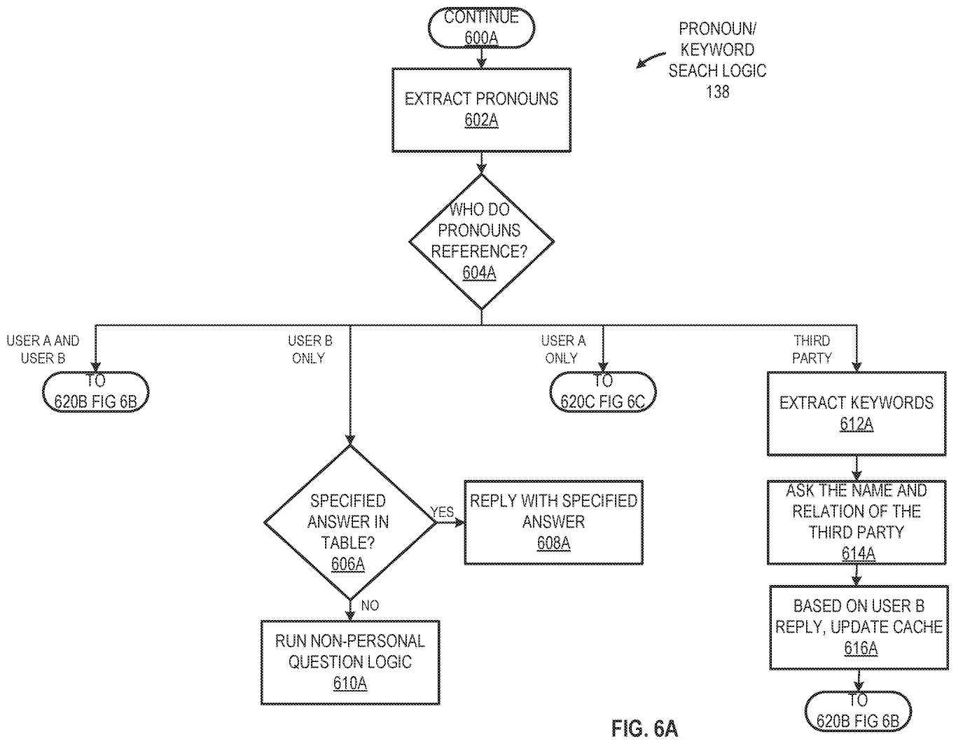

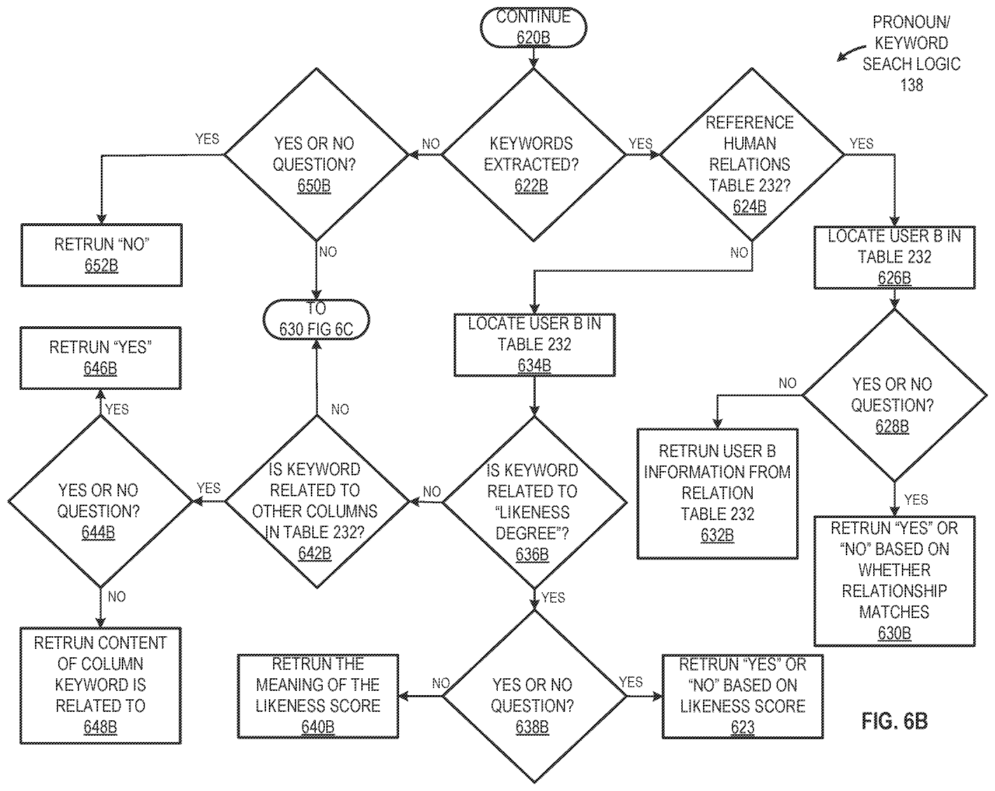

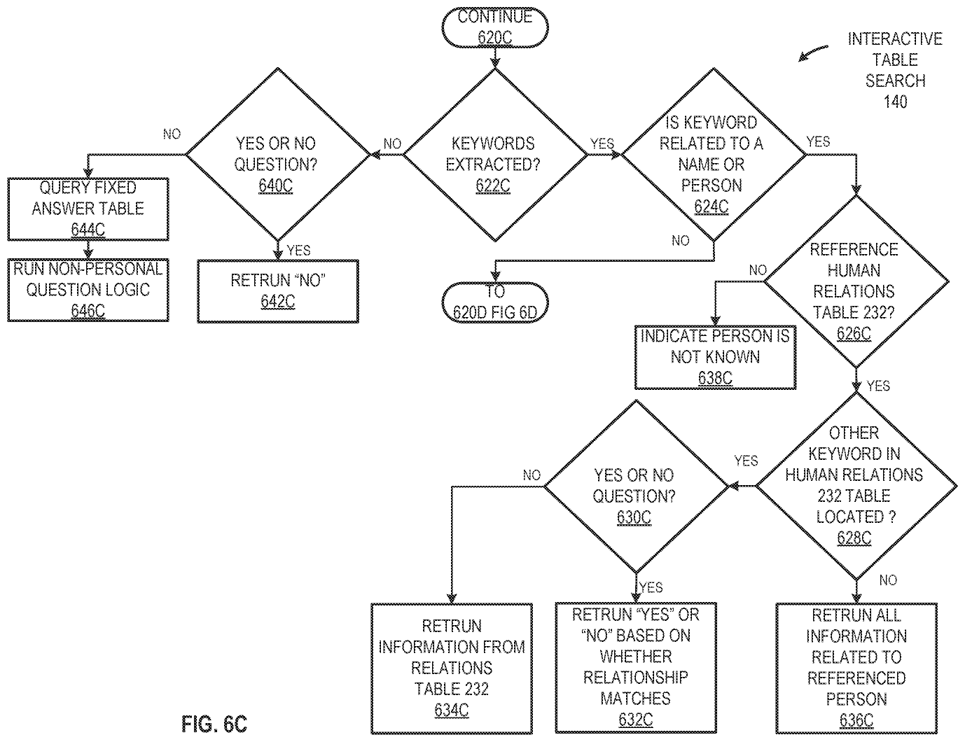

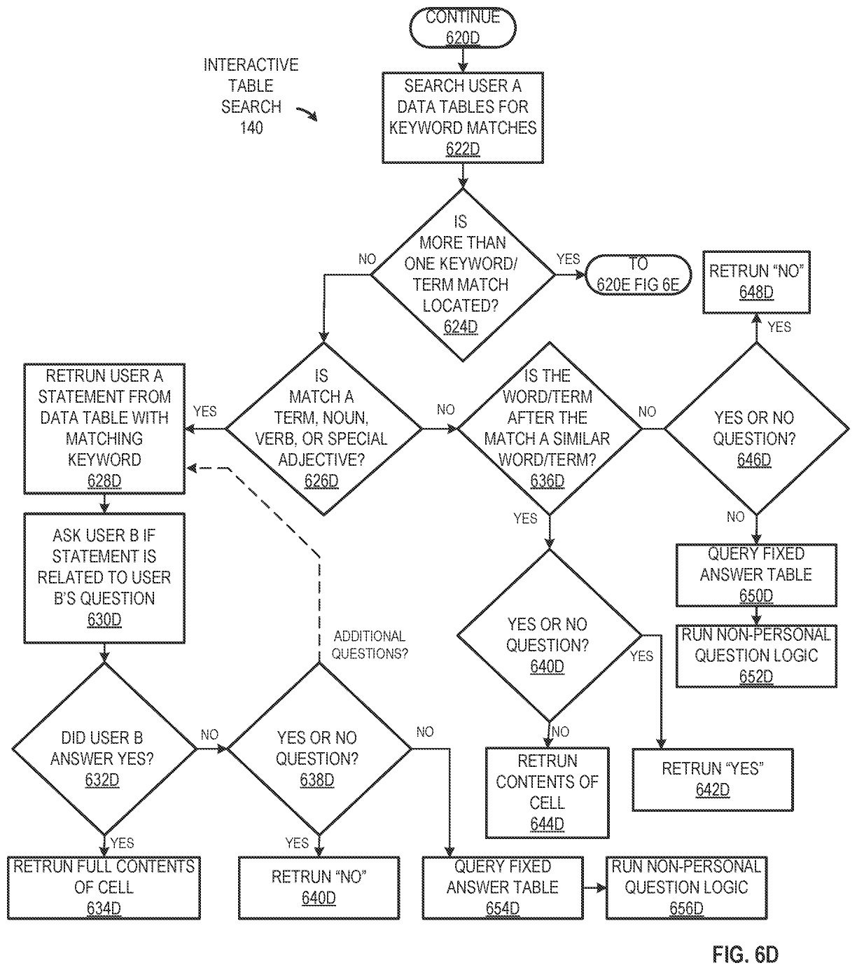

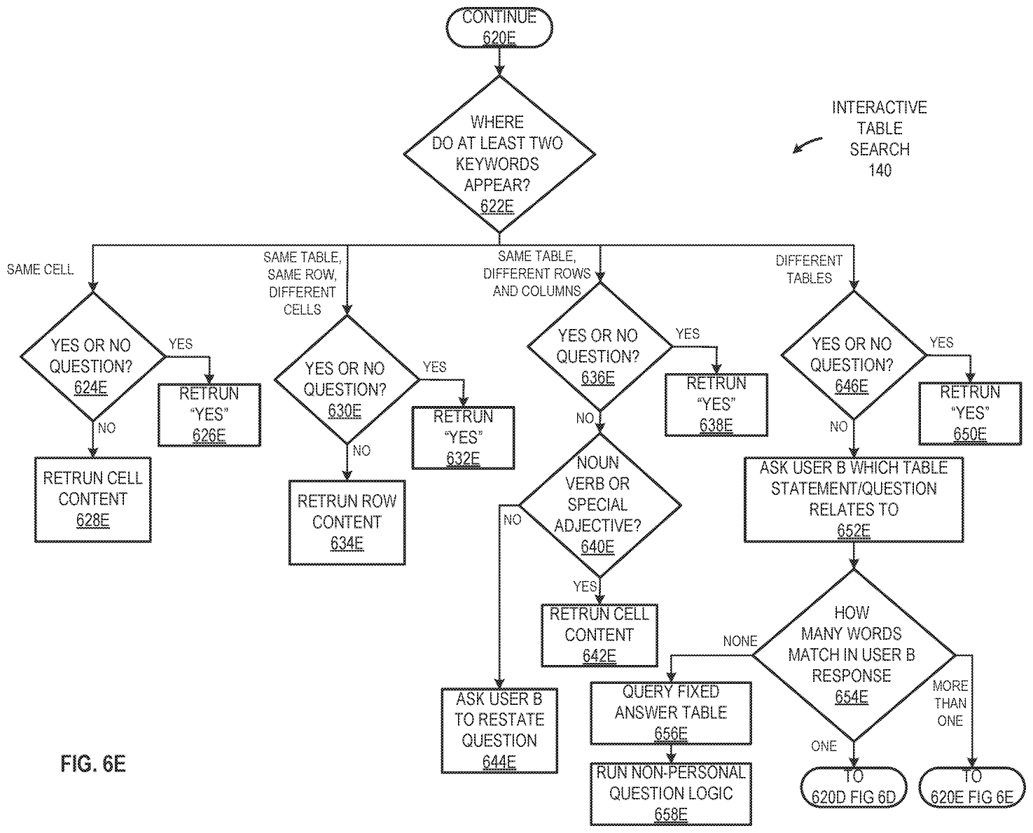

Conversely, if the statement contains a string of words comprising “you” or “your” with no other pronouns, negative words (e.g. no, not, dis-, un- etc.), and a negative adjective (e.g. stupid) or a negative noun (e.g., fool), then it will be classified as a positive statement. Similarly, if the statement contains a string of words comprising “you” or “your” with no other pronouns negative words (e.g. no, not, dis-, un- etc.), no negative words (e.g. no, not, dis-, un- etc.), and a positive adjective (e.g., smart) or a positive noun (e.g., wisdom),), then it will be classified as a positive statement. If extraction/keyword logic 134 classifies User B's statement as a positive statement, as in the two preceding examples, then it will cause digital life system to return a response to User B. In one example, the response to a positive statement may be “thank you.” Following the determination of a positive or negative classification of User B's question and/or statement, the process continues with activation of pronoun/keyword search logic 138 at operation 600A of FIG. 6A.

### Pronoun/Keyword Search Logic

Pronoun/keyword search logic 138 embodied in FIG. 1 generally performs operations related to determining who User B is referencing based on the pronouns comprised in User B's statement and/or question. Further, in the embodiment shown in FIG. 1, pronoun/keyword search logic 138, and/or interactive table search 140, performs operations related to responding to a question from User B's based on who User B is referencing, keywords and/or terms comprised in

User B's question, and the relation of those keywords and/or terms to information comprised in User A data tables 116. For example, User B may reference both User B and User A by including both first-person pronouns (I, me, etc.) and second-person pronouns (you, your, etc.) in a question directed to the digital copy of User A. In another example, User B may reference only User B or User A by including either first-person pronouns (I, me, etc.) or second-person pronouns (you, your, etc.), respectively, in a question directed to the digital copy of User A. Finally, User B may reference a third party or parties by using third-person pronouns (he, she, they, etc.) in a question directed to the digital copy of User A. Based on who User B's question is referencing, pronoun/keyword search logic 138 may further perform additional operations in order to provide a response to User B. The response may be based on comparing keywords and/or terms included in User B's question to data comprised in User A data tables 116. Further, the response provided to User B may include returning the contents of specific cells, rows, and/or columns comprised in User A data tables 116. Additionally, the response may be provided in the form of text, audio, video, etc. from data stored in User A voice/video 118.

FIGS. 6A to 6B show one embodiment of pronoun/keyword search logic 138. Further, FIGS. 6C to 6E show one embodiment of the table specific search 140 comprised within pronouns/keyword search logic 138. In this example, the operations performed by pronoun/keyword search logic 138 begin at operation 600A of FIG. 6A. Next, at operation 602A, pronoun/keyword search logic 138 extracts pronouns from User B's question and/or statement. In one example, pronouns extracted from User B's question and/or statement may include pronouns “you,” “yours,” “I,” “me,” “mine,” “my,” “she,” “we,” “he,” “they,” etc.

At operation 604A, pronoun/keyword search logic determines whether User B is referencing both User A and User B, User B, only, User A, only, or a third party based on the pronouns extracted at operation 602A. In one example, second-person pronouns such as “you,” “your,” “yours,” “yourself,” etc. indicate that User B is referencing User A and/or the digital copy of User A. Further, first-person, signal pronouns such as “I,” “me,” “mine,” “myself,” etc. indicate that User B is referencing him/herself (User B). Finally, third-person pronouns such as “he,” “him,” “his,” “himself,” “she,” “her,” “hers,” “herself,” “they,” “them,” “their,” “theirs,” “themselves,” etc. indicate that User B is referencing a third party or third parties. If User B's question and/or statement references both User A and User B, then process continues at 620B of FIG. 6B. User B's question and/or statement may reference both User A and User B when it comprises both first-person singular pronouns and second person pronouns (e.g. “do you like me?”), when in comprises first-person plural pronouns (e.g. “we,” “us,” “our,” “ours”), but not when in comprises phrases such as “can/could/will/would you give/help/tell/inform/ask me . . . ” If User B's question and/or statement references only User B, then the process continues at operation 606A. User B's question and/or statement may reference only User B when it comprises only first-person singular pronouns (e.g. “can I get better?”). If User B's question and/or statement references only User A, then the process activates interactive table search 140 and continues at 620C of FIG. 6C. User B's question and/or statement may reference only User A when it comprises only second-person pronouns (e.g. “what do you think about . . . ?”). Finally, if User B's question and/or statement references a third party or parties, then process continues at operation 612A. User B's question and/or statement may reference a third party or parties when it comprises third-person pronouns (e.g. “was he your friend?”).

FIG. 6B shows one embodiment of the operations executed by pronouns/keyword search logic 138 when User B's statement and/or question is determined to reference both User A and User B. In this example, operations begin at 620B. Next, at operation 622B, pronoun/keyword extraction logic 138 determines whether extraction/keyword logic 134 extracted at least one keyword and/or term from User B's question and/or statement at operation 510. If there is at least one keyword and/or term in User B's question and/or statement at operation, the process continues to operation 624B. If there is not at least one keyword and/or term in User B's question and/or statement at operation, the process continues to operation to operation 650B.

At operation 624B, human relation table 624B is activated and pronoun/keyword extraction logic 138 determines synonyms for the at least one keyword and/or term. This operation may be performed using a thesaurus or similar database. If the at least one keyword, term, and/or synonym is a word related to and/or referencing a family member, relative, and/or friend (e.g., “Am I your sister?”), then the process continues to operation 626B. At operation 626B pronoun/keyword search logic 138 and/or preprocessing 132 locates User B in human relationship table 232 and identifies User B's relation to User A.

Next, at operation 628B, and pronoun/keyword extraction logic 138 determines whether User B's statement and/or question was identified by extraction/keyword logic 134 as a yes or no question type. If User B's statement is a yes or no question type, then pronoun/keyword extraction logic 138 will return a “yes” response to User B if the appropriate cell in human relation table 232 contains content matching a word related to and/or referencing a family member, relative, and/or friend (“e.g., sister”) at operation 630B. For example, if User B's question was “Am I your sister?, and human relation table 138 indicates that User B is User A's sister, then pronoun/keyword extraction logic 138 will return a “yes” response. Conversely, if User B's statement is a yes or no question type, but the appropriate cell in human relation table 138 does not contain content matching a word related to and/or referencing a family member, relative, and/or friend (“e.g., sister”), then pronoun/keyword extraction logic will return a “no” response at operation 630B. For example, if User B's question was “Am I your sister?, and human relation table 138 indicates that User B is not User A's sister, then pronoun/keyword extraction logic 138 will return a “no” response.

Returning to operation 628B, if User B's statement and/or question is not a yes or no question type (e.g., “Where did we meet for the first time?), then pronoun/keyword extraction logic 138 returns the cell contents of the current row (User B's corresponding row in human relationship table 232) and column corresponding to User B's question type (e.g., the location/where question type) at operation 632B. Further, pronoun/keyword extraction logic 138 may return additional human relation table 232 cell contents based on additional keywords and or terms identified by extraction/keyword logic 134.

Returning to operation 624B, if the at least one identified keyword, term, and/or synonym is not a word related to and/or referencing a family member, relative, and/or friend (e.g., “Am I your sister?”), then the process continues to operation 634B. At operation 634B, pronoun/keyword search logic 138 and/or preprocessing 132 locates User B's row in human relationship table 232 and locates a relevant column based on the extracted keywords, terms, synonyms, and/or question type (e.g., “job” column, or the “likeness degree” column).

Next, at operation 636B, if the keywords, terms, synonyms, and/or question type caused pronoun/keyword search logic 138 to locate the “likeness degree” column, the process will continue to operation 638B. At operation 638B, if User B's statement and/or question is a yes or no question type, then pronoun/keyword extraction logic 138 returns will return either a “yes” response or a “no” response based on the likeness score located in human relations table 232. Likeness score may generally indicate User A's feelings towards User B. For example, if the likeness score is greater than 0, pronoun/keyword extraction logic 138 may return “yes.” Further, the likeness score is less than 0, then pronoun/keyword extraction logic 138 may return “no.” Finally, if the likeness score is equal to 0, the pronoun/keyword extraction logic 138 may return “I need to know more about you.” Returning to operation 638, if User B's statement and/or question is not a yes or no question type, then, at operation 640B, pronoun/keyword extraction logic 138 returns meaning associated with User A's likeness score for User B. For example, the likeness scores may range from −4 to +4. A score closer to +4 may be associated with a more positive response, and a score closer to −4 may be associated with a more negative response.

Returning to operation 636B, if the keywords, terms, synonyms, and/or question type did not cause the pronoun/keyword search logic 138 to locate the “likeness degree” column, the process will continue to operation 642B. Further, at operation 642B if the keywords, terms, synonyms caused the pronoun/keyword search logic 138 to locate columns other than the “likeness degree” column, the then process will continue at operation 644B. At operation 644B, if User B's statement and/or question is a yes or no question type, then the process will continue to operation 646B where pronoun/keyword extraction logic 138 returns a “yes” response. At operation 644B, if User B's statement and/or question is not a yes or no question type, then the process will continue to operation 648B where pronoun/keyword extraction logic 138 will return a response based on the contents of the cell in human relations table 232 that is associated the located column. Returning to operation 642B, if the keywords, terms, synonyms did not cause the pronoun/keyword search logic 138 to locate any columns, the then process will activate interactive table search 140 and continue at operation 630 of FIG. 6C.

Returning to operation 622B, if the pronoun/keyword extraction logic 138 determines that the extraction/keyword logic 134 did not extract at least one keyword and/or term from User B's question and/or statement at operation 510, then the process continues to operation 650B. At operation 650B, if User B's statement and/or question is a yes or no question type, then the process will continue to operation 652B where pronoun/keyword extraction logic 138 returns a “no” response. Conversely, if User B's statement and/or question is not a yes or no question type, then the process will activate interactive table search 140 and continue at operation 630 of FIG. 6C.

Returning to operation 604A of FIG. 6A, pronoun/keyword search logic 138 may determine that User B's statement and/or question is referencing only User B, in which case the process continues at operation 606A. At operation 606A, pronoun/keyword search logic 138 may activate basic answer logic and search a one-to-one specified answer table. In one embodiment of the current invention, the one-to-one specified answer table may be valuable things/freestyle table 244. If there are keywords, terms, and/or synonyms in User B's statement and/or question that are associated with entries in the one-to-one specified answer table, then at operation 608A, the pronoun/keyword search logic 138 will return a response based on the specific entry located in the one-to-one specified answer table. Conversely, if there are no keywords, terms, and/or synonyms in User B's statement and/or question that are associated with entries in the one-to-one specified answer table, then the process will continue at operation 610A. At operation 610A pronoun/keyword search logic 138 may activate non-personal question logic 610A. Non-personal question logic may perform a web-based search based on User B's question and/or statement. Further, non-personal question logic may perform a non-personal table search based on User B's question and/or statement.

Returning again to operation 604A of FIG. 6A, pronoun/keyword search logic 138 may determine that User B's statement and/or question references only User A, in which case interactive table search 140 is activated and the process continues at operation 620C of FIG. 6C.

### Interactive Table Search

FIG. 6C shows one embodiment of the operations executed by interactive table search 140 when User B's statement and/or question is determined to reference User A only. In this example, operations begin at 620C. Next, at operation 622C, interactive table search 140 determines whether extraction/keyword logic 134 extracted at least one keyword and/or term from User B's question and/or statement at operation 510. If there is at least one keyword and/or term in User B's question and/or statement at operation, the process continues to operation 624C. If there is not at least one keyword and/or term in User B's question and/or statement at operation, the process continues to operation to operation 640C.

At operation 640C, interactive table search 140 determines whether User B's statement and/or question is a yes or no question type. Alternately, there may be multiple stored questions and/or statements from User B. In that case, interactive table search 140 determines whether

User B's first statement and/or question is a yes or no question type. If a yes or no question type is determined, then the process continues to operation 642C and interactive table search 140 will return a “no” response. Conversely, if User B's statement is not a yes or no question type, then the process continues to operation 644C where interactive table search may activate basic answer logic and search a one-to-one specified answer table. In one embodiment of the current invention, the one-to-one specified answer table may be valuable things/freestyle table 244. If there are keywords, terms, and/or synonyms in User B's statement and/or question that are associated with entries in the one-to-one specified answer table, then interactive table search will return a response based on the specific entry located in one-to-one specified answer table. Conversely, if there are no keywords, terms, and/or synonyms in User B's statement and/or question that are associated with entries in the one-to-one specified answer table, then the process will continue at operation 646C. At operation 646C interactive table search may activate non-personal question logic. Non-personal question logic may perform a web-based search based on User B's question and/or statement. Further, non-personal question logic may perform a non-personal table search based on User B's question and/or statement.

At operation 624C, after there is at least one keyword and/or term in User B's question and/or statement identified at operation 622C, interactive table search 140 determines if one of the keywords and/or terms extracted by the extraction/keyword logic 134 is a word related to and/or referencing a family member, relative, and/or friend. In another embodiment, interactive table 140 search may extract and analyze keywords. For example, keywords may be extracted from User B's statement and/or question and analyzed according to how many keywords were extracted. Further, interactive table search 140 may identify and store the types of questions the User B provided. If one or more of the keywords and/or terms extracted from User B's statement and/or question is a word related to and/or referencing a family member, relative, and/or friend (“What is the job of your aunt?,” “What is your aunt's job?,” etc.), then process continues to operation 626C. Conversely, if one of the keywords and/or terms is not a word related to and/or referencing a family member, relative, and/or friend, then process continues to operation 620D of FIG. 6D.

At operation 626C, interactive table search 140 searches human relations table 232 for the word related to and/or referencing a family member, relative, and/or friend. If interactive table search 140 determines that neither this word nor its synonyms is related to a row in human relations table 232, then at operation 638C, User B is provided with a response indicating that the person in User B's statement and/or question is unknow. For example, interactive table search 140 may return “I am sorry, I do not know this person or the person who created this digital copy did not enter this person's information. Can you say something else? Thank you.” Conversely, if the word related to and/or referencing a family member, relative, and/or friend or one of its synonyms does not relate to a row in human relations table 232, interactive table search 140 determines which row the word relates, a question type for User B's statement, and identifies other keywords and or terms determined by preprocessing 132 then continues to operation 628C.

Next, at operation 628C, interactive table search 140 searches for a related column within human relations table 232 based on other keywords and or terms determined by preprocessing 132 (e.g., “job”). If interactive table search 140 locates a column (e.g., “job” column) in the human relations table 232, the process continues at operation 630C. If User B's statement and/or question is a yes or no question type (e.g. “Is your aunt a nurse?”), then the process continues at operation 632C and interactive table search 140 returns a response of either “yes” or “no” based on whether the content of the located cell matches with the identified keyword and/or term. For example, User B may ask “Is your aunt a nurse?” and the content of the located cell may indicate “healthcare worker.” Interactive table search 140 may determine if there is a match between the question and the cell content. A match may be determined using a score generated by WordNet. If table search 140 determines a match, then a “yes” response may be returned. Conversely, if a match is not determined, then a “no” response may be returned. Returning to operation 630C, if User B's statement and/or question is not a yes or no question type (e.g. “What is your aunt's job?”), then interactive table search 140 returns a response based on the content of the located cell.

Returning to operation 628C, if interactive table search 140 does not locates a column in the human relations table 232 based on other keywords and or terms determined by preprocessing 132 (e.g., “Can you tell me some information about your brother?”), then the process continues at operation 636C. At operation 636C, interactive table search 140 returns all information of that row related to the person referenced in User B's question and/or statement.

FIG. 6D shows one embodiment of the operations executed by interactive table search 140 when none of the keywords and/or terms extracted from User B's statement and/or question is a word related to and/or referencing a family member, relative, and/or friend as determined by operation 624C of FIG. 6C. For example, User B may ask “Did you ride a high speed train before?” or “Which school did you attend?” In those cases, keywords such as “ride,” “high, “speed,” and “train” as well as “school” and “went” may be extracted. The process begins at operation 620D. Next, at operation 622D, interactive table search 140 will search the extracted keywords, terms, and their synonyms for matches to entries in User A data tables 116. In one embodiment, interactive table search 140 will search User A data tables in the following order: 1) human relations table 232; 2) favorites table 234; 3) life experience table 230; 4) personal information table 240; 5) attitude/beliefs/summaries table 244; 6) dreams/regrets/hopes table 236; 7) needs/personality table 242; 8) valuable things/freestyle table 244.

Next, at operation 624D, interactive table search 140 determines if more than one keyword and/or term was matched to entries in User A data tables 116. If more than one keyword was matched, then the process continues at operation 620E of FIG. 6E. Conversely, if only one keyword, term, or synonym was matched to only one word or term in only one cell of all of the User A data tables 116 (e.g., “high” in the question “Did you ride a high speed train before?”, only matches “high school” in life experience table 230), then the process continues at operation 626D.

At operation 626D, interactive table search 140 determines if the matched word is a term, a solid noun (e.g., “school” rather than a non-solid noun or term, e.g., type (of)/sort of/kind/thing etc.), a verb, or a special adjective (e.g., happy, luckily, joyful, unhappy, sad, heart-broken, horrible, terrible, regrettable, fantastic, surprising, etc.) then the process continues at operation 628D. If there are multiple solid nouns, verbs, and/or special adjectives, then the nouns, verbs, and/or adjectives are ranked based the on frequency that they appear in User B's question and/or statement, wherein words appearing with lower frequency are ranked before words appearing with higher frequency. Conversely, if interactive table search 140 determines that the matched word is a regular adjective, then the process continues to operation 636D.

Next, if a term, solid noun, verb or special adjective is identified at operation 626D, then interactive table search 140 returns a response based on the statement/entry in the specific table related to the matched and/or ranked keyword at operation 628D. For example, if a keyword matched to a statement/entry in life experience table 230, interactive table search 140 may return a response identifying past life events including the certain time period, location, and type of event etc. After returning, at operation 630D, the response to User B, interactive table search 140 asks User B whether the returned response is related to User B's previous statement and/or question. For example, at 630D, interactive table search 140 may return a response to User B stating “[Relationship word (from preprocessing module, e.g., “Sister”) or name of User B], is what you said is related to my previous statement?”

Continuing to operation 632D, if User B responds affirmatively (e.g. “yes”), then interactive table search will return the full contents of the matched cell at operation 643D. For example, interactive table search may return “In that the case, your previous question, my answer is: return the contents of the table unit (cell).” Conversely, if User B responds negatively (e.g. “no”), then at operation 638D, interactive table search 140 determines whether User B's statement is a yes or no question type. If User B's question statement is a yes or no question type, then interactive table search 140 will return a “no” response to User B at operation 640D. For example, interactive table search 140 may return “In this case, your previous question, my answer is: No.” If User B has submitted multiple questions and/or statements, and the first question and/or statement is not a yes or no question type, the process returns to operation 628D. At operation 628D, interactive table search 140 repeats the process of determining whether User B's second statement and/or question contains term, solid noun, and/or special adjective that matches to a table within User A data tables 116. In some embodiments, if the second statement and/or question again reaches operation 638D, and it is again determined that the statement and/or question is not a yes or no question type, the process may again return to operation 628D if User B's has submitted a third question and/or statement.

Returning to operation 638D, if User B's question and/or statement is not a yes or no question type, and there are no additional questions and/or statements submitted by User B, then the process continues to operation 654D where interactive table search may activate basic answer logic and search a one-to-one specified answer table. If there are keywords, terms, and/or synonyms in User B's statement and/or question that are associated with entries in one-to-one specified answer table, then interactive table search will return a response based on the specific entry located that table. Conversely, if there are no keywords, terms, and/or synonyms in User B's statement and/or question that are associated with entries in the one-to-one specified answer table, then the process will continue at operation 656D. At operation 656D interactive table search may activate non-personal question logic. Non-personal question logic may perform a web-based search based on User B's question and/or statement. Further, non-personal question logic may perform a non-personal table search based on User B's question and/or statement.

Returning to operation 626D, if interactive table search 140 determines that the matched word is a regular adjective (e.g., “high” rather than a term, solid noun, or special adjective), then the process continues to operation 636D. At operation 636D, interactive table search determines if the word or term in User B's statement or question after the regular adjective is a synonym (or similar term) of a term after a regular adjective in one of the User A data tables 116. For example, if User B asks a question “Did you have a high GPA at school?,” then “GPA” is the word after the regular adjective “high.” In one of the User A data tables 116, “academic performance” may be the term after “high” in the matched cell. “Academic performance” may be considered and synonym of “GPA”. If there is such a synonym match, then the process continues to operation 640D. If there is not a synonym match, the process continues to operation 646D.

At operation 640D, interactive table search 140 determines whether User B's statement and/or question is a yes or no question type. If the statement and/or question is a yes or no question type, then, at operation 642D, interactive table search 140 will return a “yes” response. Conversely, if the statement and/or question is not a yes or no question type, then, at operation 644D, interactive table search 140 will return the contents of the cell that was matched in one of the User A data tables 116.

Returning to operation, 636D, if there is no synonym match to the word following the regular adjective in User B's question and/or statement, interactive table search determines if User B's question and/or statement is a yes or no question type. If it is a yes or no question type, then interactive table search 140 returns a “no” response at operation 648D. If it is not a yes or no question type, then then the process continues to operation 650D where interactive table search may activate basic answer logic and search a one-to-one specified answer table. If there are keywords, terms, and/or synonyms in User B's statement and/or question that are associated with entries in one-to-one specified answer table, then interactive table search will return a response based on the specific entry located that table. Conversely, if there are no keywords, terms, and/or synonyms in User B's statement and/or question that are associated with entries in the one-to-one specified answer table, then the process will continue at operation 652D. At operation 652D interactive table search may activate non-personal question logic. Non-personal question logic may perform a web-based search based on User B's question and/or statement. Further, non-personal question logic may perform a non-personal table search based on User B's question and/or statement.

FIG. 6E shows one embodiment of the operations executed by interactive table search 140 when more than one keyword and/or term was matched to different tables, cells, and/or entries within the cells of in User A data tables 116 as determined by operation 624D of FIG. 6D. The process starts at operation 620E. Next, at operation 622E, based on User B's statement and/or question, interactive table search determines whether at least two matched words belong to the same cell of a table within User A data tables 116, whether at least two matched words belong to the same row of life experience table 230 or human relations table 232 but different cells, whether at least two matched words belong to the same table within User A data tables 116 but different cells, or whether at least two matched words belong to different tables within User A data tables 116.

At operation 624E, when at least two matched words of User B's statement and/or question belong to the same cell of a table within User A data tables 116, interactive table search 140 determines whether User B's statement and/or question is a yes or no question type. If it is a yes or no question type, then at operation 626E, interactive table search 140 will return a “yes” response. It is not a yes or no question type, then interactive table search 140 will return a response based on the contents of the matched cell at operation 628E.

At operation 630E, when at least two matched words of User B's statement and/or question belong to the same row of life experience table 230 or human relations table 232 but different cells, interactive table search 140 determines whether User B's statement and/or question is a yes or no question type. If it is a yes or no question type, then at operation 632E, interactive table search 140 will return a “yes” response. It is not a yes or no question type, then interactive table search 140 will return a response based on the contents the entire row at operation 634E. In some embodiments, interactive table search will generate a sentence based on the contents of the entire row.

At operation 636E, when at least two matched words of User B's statement and/or question belong to the same table within User A data tables 116 but different cells, interactive table search 140 determines whether User B's statement and/or question is a yes or no question type. If it is a yes or no question type, then at operation 638E, interactive table search 140 will return a “yes” response. It is not a yes or no question type, then at operation 640E, interactive table search 140 determines a classification for the matched words. For example, the matched words may be classified as solid nouns, verbs, and/or adjectives. If at least one matched word is a solid noun, verb, or special adjective, and User B's statement and/or question is a yes or no question type, then interactive table search 140 will return a “yes” response. If at least one matched word is a solid noun, verb, or special adjective, and User B's statement and/or question is not a yes or no question type, then interactive table search 140 will return a response based on the contents of the matched cell or cells at operation 642E. Conversely, if none of the matched words are not solid nouns, verbs, or special adjectives, then, at operation 644E, interactive table search 140 will return a response asking User B to resubmit/restate the question and/or statement. For example, interactive table search may return “Perhaps that you are talk about [statement referencing the e table that the two cells belong to], however, can you rephrase your question [and/or statement] so that I can understand it better.” After User B′ relies, the process may restart at operation 400 of FIG. 4 or operation 500 of FIG. 5.

At operation 646E, when at least two matched words of User B's statement and/or question belong to different tables within User A data tables 116, interactive table search 140 determines whether User B's statement and/or question is a yes or no question type. If it is a yes or no question type, then at operation 646E, interactive table search 140 will return a “yes” response. It is not a yes or no question type, then at operation 652E, interactive table search 140 will determine which tables of the User A data tables 116 that the solid nouns, special adjectives, and/or verb's of User B's statement and/or question belong to. Then, interactive table search will return a response to User B asking which table User B's question and/or statement belongs to. For example, interactive table search may return “I am sorry, is what you want to know is related to [statements referencing first table], [statements referencing second table], [statements referencing third table], etc.” Next, if User B responds indicating a specific table, then at operation 654E, interactive table search 116 determines how many words in User B's initial statement and/or question matched to that table. If there is only one matched word in that table, then the process returns operation 620D of FIG. 6D. In other embodiments of the current invention, the process may continue at operation 626D of FIG. D rather than operation 620D. Further, when interactive search logic 140 returns a response based on the operations followed after returning to operation 620D or 626D, a statement such as “In this case, for your previous question, my answer is” may be included prior to returning the response. Conversely, if there is more than one matched word in the table specified by User B, then the process returns operation 620E of FIG. 6E. Further, when interactive search logic 140 returns a response based on the operations followed after returning to operation 620E, a statement such as “In this case, for your previous question, my answer is” may be included prior to returning the response. Finally, if User B did not respond indicating a table, or if there are no word matches in the table referenced by User B, then then then the process continues to operation 656E where interactive table search may activate basic answer logic and search a one-to-one specified answer table. If there are keywords, terms, and/or synonyms in User B's statement and/or question that are associated with entries in one-to-one specified answer table, then interactive table search will return a response based on the specific entry located that table. Conversely, if there are no keywords, terms, and/or synonyms in User B's statement and/or question that are associated with entries in the one-to-one specified answer table, then the process will continue at operation 658E. At operation 658E interactive table search may activate non-personal question logic. Non-personal question logic may perform a web-based search based on User B's question and/or statement. Further, non-personal question logic may perform a non-personal table search based on User B's question and/or statement.

Returning again to operation 604A of FIG. 6A, pronoun/keyword search logic 138 may determine that User B's statement and/or question references is referencing a third party or third parties. If such a determination is made, then the process continues at 612A. At operation 612A, pronoun/keyword search 138 extracts keywords and terms from User B's. Further, the keywords and terms may be stored in a cache. Next, at operation 614A, the system will return a response asking User B to indicate the name and/or relation of the third party or parties. For example, pronoun/keyword search 138 may return “[User B's name and/or relation to User A], you said [reference to third party or parties]. Could you tell me their/his/her name(s)?” Next, at operation 616A, the cache is updated based on the name(s) and or relationship(s) indicated in User B's response. Further, the previous terms and/or keywords may be extracted from the cache. Then, the process continues at operation 620B of FIG. 6B.

### Table Specific Search Logic

Table specific search logic 136 embodied in FIG. 1 generally performs operations related to identifying and returning responses based on questions and/or statements from User B. The responses are generated based on table-specific activation terms identified in User B's question and/or statement. Further, because User A data tables 116 are used to generate responses, table specific search logical is beneficially able to respond to questions and/or statements as User A. Moreover, because personal information and memory collection logic may prompt User A to provide more extensive, detailed, and specific information that User A might otherwise provide in a typical human to human interaction, User A data tables 116 may contain more extensive, detailed, and specific information about User A's life that User A would typical have access to in a typical human conversation or interaction. Thus, table specific logic 136 may beneficially enable a digital copy of User A to provide more extensive, detailed, and specific information about User A in response to questions from User B than would otherwise be possible in a typical human to human conversation or interaction. Specially, table specific search logic 136 may identify specific tables related to questions and/or statements submitted by User B based on table specific activation terms identified by User B. For example, User B's statement and/or question may include activation terms related to any one of the User A data tables 116. Table specific search logic identifies which table the activation terms are related to and further provides a response based on the information stored in that specific table.

FIG. 7A shows one embodiment of table specific search logic 136. In this example, the operations performed by table specific search logic 136 begin at operation 700A of FIG. 7A. Next, at operation 702A, table specific search logic 136 determines which table of the User A data tables is activated based on the table-specific activation terms identified by preprocessing 132. If the activation term is related to human relations table 232, the process continues at operation 620B of FIG. 6B. In other embodiments, when the activation term is related to human relations table 232, then the process may continue at operation 620B or 620C depending on the pronouns appearing in User B's statement and/or question. For example, User B submits a question as a string of words comprising “I, me, my, mine, or myself” and “you, your, yours, or yourself” (e.g., “Do you like me?”), or if the question has a string of words comprising only the words “we or us” with no other pronouns, but not if the question has a string of words comprising “can, could, will, or would” and “you give, help, tell, inform, or ask me”, then the process may continue at operation 620B. Conversely, if one of the keywords or terms in User B's statement and/or question is a family member, relative, friend or a person's name, then the process may continue at operation 620C of FIG. 6C. If the activation term is related to life experience table 230, the process continues at operation 720B of FIG. 7B. If the activation term is related to favorites table 234, the process continues at operation 620C of FIG. 7C. If the activation term is related to attitude/beliefs/summaries table 238, the process continues at operation 720D of FIG. 6B. If the activation term is related to dreams/regrets/hopes table 236, the process continues at operation 720E of FIG. 7E. If the activation term is related to personal information table 240, the process continues at operation 720F of FIG. 7F.

FIG. 7B shows one embodiment of the operations executed by table specific search logic 136 when User B's question and/or statement comprises table-specific activation terms related to life experience table 230. The process starts at operation 720B. Next, at operation 722B table specific search logic 136 determines whether User B's statement and/or question contains location or age-related terms. For example, a location-related term may be the name of a city. Further, an age-related term might be a reference to a specific time period during User A or User B's life, for example childhood, child, etc. If User B's question and/or statement contains neither location nor age-related terms, then the process continues at operation 724B. If the question and/or statement contains a location-related term, the process continues at operation 750B. Finally, if the question and/or statement contains an age-related term, the process continues at operation 758B.

At operation 724B, when User B's question and/or statement contains neither location nor age-related terms, table specific search logic 136 determines whether there are any words in User B's question and/or statement that match keywords and/or terms in life experience table 230. If there is exactly one matched keyword or term, the process continues at operation 726B. If there are no matched keywords and/or terms, then the process continues at operation 748B. Finally, if there are more than one matched keywords and/or terms, then the process continues at operation 750B.

At operation 726B, when only one keyword or term from User B's statement is matched, table specific search logic determines if the matched word is a term, a solid noun (e.g., “school” rather than a non-solid noun or term, e.g., type (of)/sort of/kind/thing etc.), a verb, or a special adjective (e.g., happy, luckily, joyful, unhappy, sad, heart-broken, horrible, terrible, regrettable, fantastic, surprising, etc.). Next, if a term, solid noun, verb or special adjective is identified at operation 626D, then table specific search logic 728B returns a response based on the cell contents of the matched keyword and/or term at operation 728D. Conversely, if table specific search logic 136 determines that the matched word is a regular adjective (e.g., “high” rather than a term, solid noun, or special adjective), then the process continues to operation 730B. At operation 730B, table specific search logic 136 determines if the word or term in User B's statement and/or question after the regular adjective is a synonym (or similar term) of the word that follows that regular adjective in life experience table 230. For example, if User B asks a question “Did you have a high GPA at school?,” then “GPA” is the word after the regular adjective “high.” In life experience table 230, “academic performance” may be the term after “high” in the matched cell. “Academic performance” may be considered and synonym of “GPA.” If there is such a synonym match, then the process continues to operation 732B. If there is not a synonym match, the process continues to operation 740B.

At operation 732B, table specific search logic 136 determines whether User B's statement and/or question is a yes or no question type. If the statement and/or question is a yes or no question type, then, at operation 734B, table specific search logic 136 returns a “yes” response. Conversely, if the statement and/or question is not a yes or no question type, then, at operation 736B, table specific search logic 136 will return the contents of the cell that was matched life experience table 230.

Returning to operation, 730B, if there is no synonym match to the word following the regular adjective in User B's question and/or statement, table specific search logic 136 determines whether User B's question and/or statement is a yes or no question type. If it is a yes or no question type, then table specific search logic 136 returns a “no” response at operation 742B. If it is not a yes or no question type, then then the process continues to operation 744B where table specific search logic 136 may activate basic answer logic and search a one-to-one specified answer table. If there are keywords, terms, and/or synonyms in User B's statement and/or question that are associated with entries in one-to-one specified answer table, then interactive table search will return a response based on the specific entry located that table. Conversely, if there are no keywords, terms, and/or synonyms in User B's statement and/or question that are associated with entries in the one-to-one specified answer table, then the process will continue at operation 746B. At operation 746B interactive table search may activate non-personal question logic. Non-personal question logic may perform a web-based search based on User B's question and/or statement. Further, non-personal question logic may perform a non-personal table search based on User B's question and/or statement.

Returning to operation 724B, if there are no matched keywords and/or terms in User B's statement, then the process continues at operation 748B. At operation 748B, table specific search logic 136 searches for synonyms of keywords and/or terms that may be in User B's statement and/or question. For example, table specific search logic 136 may search for 3 to 4 synonyms. If one or more synonyms of a keyword and/or term is identified in User B's question and/or statement, the then the process continues to operation 750B. If no synonyms of a keywords and/or term is identified, then the process continues to operation 754B.

At operation 754B, table specific search logic 136 determines if User B's question and/or statement contains a solid noun. For example, in the question “have you ever planted a flower when you were young?”, the word “flower” is a solid noun. Based on the solid noun, table specific search logic 136 searches for upper level words related to the same “type” as the solid noun. In one embodiment, table specific search logic 136 may use Conceptnet.io to search for upper level matches using “a type of” search. For example, Conceptnet.io might identify “plant” as an upper level match to “flower.” In other words, “flower” is “a type of” “plant.” If an upper level match is identified, then table specific search logic 136 searches for matching keywords and/or terms in life experience table. If a match is located between the upper level word and a keyword and/or term in life experience table 230, then, at operation 752B, table specific search logic 136 may return a response based on the content of the matched cell in life experience table 230. For example, table specific search logic 136 may return a response such as “I know you hope to know my [life experience] related to [solid noun, e.g. “flower]. I cannot directly recall that, but I know [solid noun, e.g. “flower”] is a type of [upper level word, e.g. “plant”]. My [life experience] related to [upper level word, e.g. “plant”] is [content of the cell with a keyword matching the upper level word].”

Conversely, if there is not a match between the solid noun's upper level words (e.g., plant) and words in the activated table, then using the upper level word, table specific search logic 136 may to search for “Words as Types of [the solid noun's upper level word].” For example, “tree” is a type of “plant.” Thus “tree” is a “type of” “flower's” upper level word “plant.” Again, if a word is identified using the “Words as Types of [the solid noun's upper level word” search, then table specific search logic 136 searches for matches between that word and keywords and/or terms associated with life experience table 230. If such a keyword and/or term is located, then table specific search logic 136 may return a response based on the cell associated with that keyword and/or term. For example, table specific search logic 136 may return “I know you hope to know my [life experience] related to [solid noun, e.g. “flower”]. I cannot directly recall that, however, I know both [solid noun, e.g. “flower”] and [word that is the type of the solid noun's upper level word, e.g “tree”] belong to [solid noun's upper level word, e.g. “plant”] and my personal experience related to [word that is the type of the solid noun's upper level word, e.g “tree”] is [content of the cell with a keyword matching word that is the type of the solid noun's upper level word, e.g “tree”].

Returning to operation 754B, if an upper level word is not identified, or if the identified upper level word does not return a match in the life experience table, or if a type of the upper level word is not identified, or if the type of the upper level word does not return a match in the life experience table, then the process continues at operation 756B. Based on the solid noun identified in operation 754B, table specific search logic 136 searches for lower level words that are a “type” of that solid noun. In one embodiment, table specific search logic 136 may use Conceptnet.io to search for lower level matches using “words as types of [solid noun]” search. For example, if the sold noun is “plant,” Conceptnet.io may identify “tree” as a type of “plant.” If such a lower lever term is identified, table specific search logic searches for keyword and/or term matches in life experience 230. If a match is located between the lower level word and a keyword and/or term in life experience table 230, then, at operation 752B, table specific search logic 136 may return a response based on the content of the matched cell in life experience table 230. For example, table specific search logic 136 may return a response such as “I know you hope to know my [life experience] related to [solid noun, e.g. “plant”]. I cannot directly recall that, but I know [lower level word, e.g. “tree”] is a type of [solid noun, e.g. “plant”]. My [life experience] related to [lower level word, e.g. “tree”] is [content of the cell with a keyword matching the lower level word].”

If no lower level or upper level matches are identified, then the process continues at operation 746B. At operation 746B, table specific search logic 136 determines whether User B's question and/or statement is a yes or no question type. If it is a yes or no question type, then, at operation 742B, table specific search logic 136 returns a “no” response. If it is not a yes or no question type, then, at operation 748B, table specific search logic 136 returns a response indicating that no record is available. for example, table specific search logic 136 may return a response such as “I know that you hope to know something about my [life experience]. Unfortunately, it seems that the real [User A's name] did not record this specific information.”

At operation 750B, when User B's question and/or statement contains a location-related term as determined in operation 722B, table specific search logic 136 identifies whether User B's statement and/or question contains an keywords and/or terms associated with specific cells in life experience table 232. Similarly, returning to operation 724B, if there are more than one matched keywords and/or terms in User B's statement, then the process continues also continues at operation 750B by identifying keywords and or terms associated with specific cells in life experience table 232. Further, if synonyms of keywords and or terms are identified in terms in User B's statement, then the process continues also continues at operation 750B. If table specific search logic 136 identifies a direct match to a cell within life experience table 232 based on such keywords and/or terms, then a response based on that cell's content is returned to User B. If no direct match is located. If no direct matches to a cells within life experience table 232 are located based on the identified keywords and/or terms, the process continues at operation 754B as described above.

At operation 758B, when User B's question and/or statement contains an age-related term as identified in operation 722B, table specific search logic 136 determines an age group of the age-related term. The age group may be identified based on the following criteria: Childhood (age 0-18), key terms: “When you are/were a child/kid/in school,” “Your childhood,” etc.; Teenage/adolescent (age 12-18), key terms: “When you are/were a teenage/in middle school/in high school” etc.; Young (age 0-60), key terms: “You were/are young” etc.; Middle Age (40-60), key terms “You were/are in middle age” etc.; Old (age over 60), key terms: “You were/are old” etc.; Married (age over 20, key terms: “marriage/marry” etc. Next, at operation 760B, the specific rows related to the determined age range are activated in life experience table 230. If User A's digital copy's current age is less that the age of the determined age group, then table specific search logic 136 may return a response indicating that User A is younger than that age group. For example, table specific search logic 136 may return, “I am still young and I have experienced that age yet.”

At operation 762B, table specific search logic 136 searches for keywords and/or terms from User B's question and/or statement that match the rows in life experience table 230 activated based on the determined age group. If there is a cell with keywords and/or terms that match words in User B's question and/or statement, then table specific search logic 136 returns a response based on the content of the cell. If User B's question and/or statement contains no keywords matching cells within the activated row(s), then table specific search logic 136 returns a response based on the content of the entire activated row(s).

FIG. 7C shows one embodiment of the operations executed by table specific search logic 136 when User B's question and/or statement comprises table-specific activation terms related to favorites table 234. The process starts at operation 720C. Next, at operation 722C table specific search logic 136 extracts the words after the term or word from User B's question and/or statement that activated favorites table 234. For example, User B may submit a question with the words “like,” “enjoy,” “favorite,” etc. that activate favorites table 234. Such a question may be “do you like playing poker?” from which table specific search logic 136 will extract the words “playing poker.” Further, at operation 722C table specific search logic 136 determines whether there are negative words in User B's question and/or statement. For example, “not,” “dis,” “do not,” “don't,” etc. may be negative words in a question such as “what kind of food don't you like to eat?” If there are no negative words in User B's question and/or statement, then the process continues at operation 726C. If there are negative words, then the process continues at operation 724C.

At operation 724C, when User B's question and/or statement contains negative words, table specific search logic 136 determines what type of sentence structure the negative words appear in. For example, one type of sentence structure that contains negative words may be “what kind/type of [thing] don't you like?” If this type of sentences structure is determined, then the process continues at operation 726C, but the phrase “I don't like others” is added to the end of the response submitted to User B. Another type of sentence structure may be “don't you like [thing]” or “you don't like [thing], right?” If this type of sentences structure is determined, then the process continues at operation 726C, but the phrase User B's question will be treated as a yes or no question type.

At operation 726C, table specific search logic 136 determines if User B's question and/or statement contains a solid noun. For example, as explained above in relation to operation 754B, in the question “do you like flowers?”, the word “flower” is a solid noun. If a solid noun is determined, then table specific search logic 136 further determines if the solid noun matches any keyword and/or term in favorites table 234. If a matched is determined, the process continues to operation 728C. If a match is not determined, the table specific search logic 136 will search for synonymous of the identified solid noun that match keywords and/or terms in favorites table 234. If synonyms are matched, then the process continues to operation 728C. If there are no matches, then the process continues to operation 734C.

At operation 728C, table specific search logic 136 determines a degree of match between the adjective describing the identified solid noun and the adjective describing that same solid noun in the cell of favorites table 234. If the degree of match is low then the process continues to operation 732C. A low degree of match may be, for example, the adjective noun combination “green tea” in User B's question and “red tea” in favorites table 234. At operation 732C, when the degree of match is low, then table specific search logic 136 will return a “no” response if User B's question is a yes or no question type. If User B's question is not a yes or no question, then table specific search logic 136 will return “I like [contents of the cell in favorites table 234], but not [favorite term in User B's question].” At operation 730C, when the degree of match is high, then table specific search logic 136 will return a “yes” response if User B's question is a yes or no question type. If User B's question is not a yes or no question, then table specific search logic 136 will return “I like [contents of the cell in favorites table 234].”

At operation 734C, when a solid noun match is not determined at operation at operation 726C, table specific search logic 136 will run an upper level search similar to the upper level search described for operation 754B. For example, if an upper level match is identified, then table specific search logic 136 searches for matching keywords and/or terms in favorites table 234. If a match is located between the upper level word and a keyword and/or term in favorites table 234, then, at operation 736C, table specific search logic 136 may return a response based on the content of the matched cell in favorites table 234. The response may be modified based on the identified upper level match and the content of the matched cell in a way similar to the modified response described at operation 752B when an upper level match is identified in life experience table 230. For example, table specific search logic 136 may return a response such as “I am not sure if I like [favorite term from User B's question], but I know [favorite term from User B's question] is a kind of [upper level match]. I like [matched cell's content].” If an identified upper level word does not match a keyword or term in favorites table 234, the table specific search 136 may search identify a type of the upper level word and search for matches in favorites table 234 based on the type of upper level word following a similar process as described in operation 754B.

Returning to operation 734C, if an upper level word is not identified, or if the identified upper level word does not return a match in favorites table 234, or if a type of the upper level word is not identified, or if the type of the upper level word does not return a match in favorites table 234, then the process continues at operation 738C. Based on the solid noun identified in operation 726C, table specific search logic 136 searches for lower level words that are a “type” of that solid noun, similar to the process described at operation 756B. If such a lower lever term is identified, table specific search logic searches for keyword and/or term matches in favorites table 234. If a match is located between the lower level word and a keyword and/or term in favorites table 234, then, at operation 736C, table specific search logic 136 may return a response based on the content of the matched cell in favorites table 234. For example, table specific search logic 136 may return a response such as “I only like one kind of [favorite term from User B's response], which is [match cell's content].”

If no lower level or upper level matches are identified, then the process continues at operation 740C. At operation 740C, table specific search logic 136 may return a response based on User A's location of living when User A was less than 16 years old. For example, table specific search logic 136 may return a response based on pre-prepared favorites for people in the area that matches User A's location of living.

FIG. 7D shows one embodiment of the operations executed by table specific search logic 136 when User B's question and/or statement comprises table-specific activation terms related to attitude/beliefs/summaries table 238. The process starts at operation 720D. Next, at operation 722D table specific search logic 136 determines whether User B's question and/or statement relates to an attituded/belief or a summary. If User B's question and/or statement relates to an attituded/belief then the process continues at operation 724D. If it relates to a summary, then the process continues at operation 746D.

At operation 724D, when User B's question and/or statement relates to an attituded/belief, then table specific search logic 136 extracts the words related to the keyword or term from User B's question and/or statement that activated attitude/beliefs/summaries table 238.

For example, User B may submit a question such as “what is your attitude/belief towards religion” from which table specific search logic 136 will extract the words “religion.” Next, at operation 726D, table specific search logic 136 searches for keywords or terms in attitude/beliefs/summaries table 238, and synonyms of keywords or terms in attitude/beliefs/summaries table 238 that match the extracted word from User B's statement that relates to a term of attitude or belief. If there is a keyword, term, or synonym match in attitude/beliefs/summaries table 238, then process continues at operation 728D where table specific search logic 136 returns a response based on the matched cell's content. If the content of the matched cell is blank, for example, if User A never submitted information related to the specific cell, then table specific search logic 136 may return a response indicating that User A did not submit information related to User B's question. Returning to operation 726D, if there are no keywords, terms, or synonyms matched in attitude/beliefs/summaries table 238, then process continues at operation 730D.

At operation 730D, when no keywords, terms, or synonyms are match to the extracted word from User B's statement that relates to a term of attitude or belief, the table specific search logic 136 will run an upper level search similar to the upper level search described for operation 754B. For example, if an upper level match is identified, then table specific search logic 136 searches for matching keywords and/or terms in attitude/beliefs/summaries table 238. If a match is located between the upper level word and a keyword and/or term in attitude/beliefs/summaries table 238, then, at operation 732D, table specific search logic 136 may return a response based on the content of the matched cell in attitude/beliefs/summaries table 238. The response may be modified based on the identified upper level match and the content of the matched cell in a way similar to the modified response described at operation 752B when an upper level match is identified in life experience table 230. For example, table specific search logic 136 may return a response such as “as far as I know [object of attitude/belief] is a kind/type of [upper level match to object of attitude/belief from Conceptnet.io], and my attitude towards the [upper level match to object of attitude/belief from Conceptnet.io] is [matched cell's content].”If an identified upper level word does not match a keyword or term in attitude/beliefs/summaries table 238, the table specific search 136 may search identify a type of the upper level word and search for matches in attitude/beliefs/summaries table 238 based on the type of upper level word following a similar process as described in operation 754B.

Returning to operation 730D, if an upper level word is not identified, or if the identified upper level word does not return a match in attitude/beliefs/summaries table 238, or if a type of the upper level word is not identified, or if the type of the upper level word does not return a match in attitude/beliefs/summaries table 238, then the process continues at operation 734D. Based on the object of attituded/belief extracted at operation 724D, table specific search logic 136 searches for lower level words that are a “type” of that extracted attituded/belief, similar to the process described at operation 756B. If such a lower lever term is identified, table specific search logic searches for keyword and/or term matches in favorites table 234. If a match is located between the lower level word and a keyword and/or term in attitude/beliefs/summaries table 238, then, at operation 732D, table specific search logic 136 may return a response based on the content of the matched cell in attitude/beliefs/summaries table 238. For example, table specific search logic 136 may return a response such as “what you mentioned is a general concept, my attitude is more specific. [return matched cell's content].”

If no lower level or upper level matches are identified, then the process continues at operation 736D. At operation 736DC, table specific search logic 136, or another logic such as general judgment/attituded logic, may determine a response based on whether the extracted object of attitude/belief is a passive thing or a positive thing, and based on whether User A's personality has a high or low selfishness score. The extracted object of attitude/belief may be a passive thing, for example, when it is related to things such as “people are starving,” “dying,” “death,” “accident,” etc. The extracted object of attitude/belief may be a positive thing, for example, when it is related to things such as “happy” etc.

If User B's question and/or statement contains an extracted object of attitude/belief that is passive, it is negatively modified (e.g. “not,” “doesn't,” “don't,” etc.), and if User A's selfishness score is relatively low, then the process continues at operation 738D. At operation 738D, table specific search logic 136 may return a response such as, “generally speaking I am sorry to hear that and I would like to help if possible.” Further, table specific search logic 136 may return, “when something is wrong, my thought is [contents of cell from attitude/beliefs/summaries table 238 related to User A's response to the question “what is your attituded or reaction towards a negative or unhappy event that happens to you?”].”

If User B's question and/or statement contains an extracted object of attitude/belief that is passive, and if User A's selfishness score is high, then the process continues at operation 740D.

At operation 740D, table specific search logic 136 may return a response such as, “generally speaking I am sorry to hear that and I would like to help if possible.” Further, table specific search logic 136 may return, “when something is wrong, my thought is [contents of cell from attitude/beliefs/summaries table 238 related to User A's response to the question “what is your attituded or reaction towards a negative or unhappy event that happens to you?”].”

If User B's question and/or statement contains an extracted object of attitude/belief that is positive, it is negatively modified (e.g. “not,” “doesn't,” “don't,” etc.), and if User A's selfishness score is relatively low, then the process continues at operation 742D. At operation 742D, table specific search logic 136 may return a response such as, “I feel happy to hear that.” Further, table specific search logic 136 may return, “generally speaking, when something good happens, my thought is [contents of cell from attitude/beliefs/summaries table 238 related to User A's response to the question “what is your attituded or reaction towards a happy or good event that happens to you?”].”

If User B's question and/or statement contains an extracted object of attitude/belief that is positive, and if User A's selfishness score is high, then the process continues at operation 744D. At operation 744D, table specific search logic 136 may return a response such as, “good to hear that.” Further, table specific search logic 136 may return, “generally speaking, when something good happens, my thought is [contents of cell from attitude/beliefs/summaries table 238 related to User A's response to the question “what is your attituded or reaction towards a happy or good event that happens to you?”].”

Returning to operation 746D, when User B's question and/or statement relates to a summary, then table specific search logic 136 extracts the words related to the keyword or term from User B's question and/or statement that activated attitude/beliefs/summaries table 238. For example, the extracted summary-related word may be “life.” Next, at operation 748D, table specific search logic 136 searches for keywords or terms in attitude/beliefs/summaries table 238, and synonyms of keywords or terms in attitude/beliefs/summaries table 238 that match the extracted word from User B's statement that relates to a summary term. If there is a keyword, term, or synonym match in attitude/beliefs/summaries table 238, then process continues at operation 728D where table specific search logic 136 returns a response based on the matched cell's content. If the content of the matched cell is blank, for example, if User A never submitted information related to the specific cell, then table specific search logic 136 may return a response indicating that User A did not submit information related to User B's question. Returning to operation 748D, if there are no keywords, terms, or synonyms matched in attitude/beliefs/summaries table 238, then process continues at operation 750D.

At operation 750D, table specific search logic 136 determines a degree of match between the extracted summary-related keyword, term, or synonym, and the most similar keyword and/or term from attitude/beliefs/summaries table 238. If the degree of match is low, then the process continues to operation 720B of FIG. 2. A low degree of match may be, for example, based on a minimal closeness score, where a minimal closeness score less than 0.5 is a low degree of match. If the degree of match is high, then the process continues at operation 752D. At operation 752D, when the degree of match is high, then table specific search logic 136 will return a response based on the contents of the cell with the most similar keyword and/or term from attitude/beliefs/summaries table 238.

FIG. 7E shows one embodiment of the operations executed by table specific search logic 136 when User B's question and/or statement comprises table-specific activation terms related to dreams/regrets/hopes table 236. The process starts at operation 720E. Next, at operation 722E, table specific search logic 136 searches for keywords or terms in dreams/regrets/hopes table 236, and synonyms of keywords or terms in dreams/regrets/hopes table 236 that match the extracted word from User B's statement that relates to a term of dreams, regrets, and/or hopes. If there is a keyword, term, or synonym match in dreams/regrets/hopes table 236, then process continues at operation 724E where table specific search logic 136 returns a response based on the matched cell's content. If the content of the matched cell is blank, for example, if User A never submitted information related to the specific cell, then table specific search logic 136 may return a response indicating that User A did not submit information related to User B's question. Returning to operation 722E, if there are no keywords, terms, or synonyms matched in dreams/regrets/hopes table 236, then process continues at operation 726E.

At operation 730D, when no keywords, terms, or synonyms are match to the extracted word from User B's statement that relates to a term of dreams, regrets, or hopes, the table specific search logic 136 will run an upper level search similar to the upper level search described for operation 754B. For example, if an upper level match is identified, then table specific search logic 136 searches for matching keywords and/or terms in dreams/regrets/hopes table 236. If a match is located between the upper level word and a keyword and/or term in dreams/regrets/hopes table 236, then, at operation 724E, table specific search logic 136 may return a response based on the content of the matched cell in dreams/regrets/hopes table 236. The response may be modified based on the identified upper level match and the content of the matched cell in a way similar to the modified response described at operation 752B when an upper level match is identified in life experience table 230. If an identified upper level word does not match a keyword or term in dreams/regrets/hopes table 236, the table specific search 136 may search identify a type of the upper level word and search for matches in dreams/regrets/hopes table 236 based on the type of upper level word following a similar process as described in operation 754B.

Returning to operation 726E, if an upper level word is not identified, or if the identified upper level word does not return a match in dreams/regrets/hopes table 236, or if a type of the upper level word is not identified, or if the type of the upper level word does not return a match in dreams/regrets/hopes table 236, then the process continues at operation 728E. Based on the term(s) of dreams, regrets, or hopes in User B's question and/or statement, table specific search logic 136 searches for lower level words that are a “type” of that dreams, regrets, or hopes, similar to the process described at operation 756B. If such a lower lever term is identified, table specific search logic searches for keyword and/or term matches in favorites table 234. If a match is located between the lower level word and a keyword and/or term in dreams/regrets/hopes table 236, then, at operation 724E, table specific search logic 136 may return a modified response based on the content of the matched cell in dreams/regrets/hopes table 236.

If no lower level or upper level matches are identified, then the process continues at operation 730E. For example, if User B's may submit a question stating, “what is your hope towards your new job?” but “new job” is not a column in dreams/regrets/hopes table 236. In this case, table specific search logic 136 will search life experience table 230, human relations table 232, favorites table 234, attitude/beliefs/summaries table 238, needs/personality table 240, and personal information table 242 for matches to the object of User A's hopes, regrets, or dreams identified in User B's statement and/or question.

If the object of User A's hopes, regrets, or dreams matches a keyword or term in a cell of the life experience table 230, then at operation 732E, table specific search logic 136 will return a response based on the matched cell. For example, table specific search logic 136 may return “my previous experience related to [object of dreams/regrets/hope] is [cell's content] and I hope things get better.”

If the object of User A's hopes, regrets, or dreams matches a keyword or term in a cell of the human relations table 232, then at operation 734E, table specific search logic 136 will return a response based on the matched cell. For example, table specific search logic 136 may return “I recall that [object of dreams/regrets/hope] is related to [relation to person or person's name from human relations table 232] and I hope things get better with [relation to person or person's name from human relations table 232].”

If the object of User A's hopes, regrets, or dreams matches a keyword or term in a cell of the favorites table 234, then at operation 736E, table specific search logic 136 will return a response based on the matched cell. For example, table specific search logic 136 may return “I like that [object of dreams/regrets/hope] and I hope things get better.”

If the object of User A's hopes, regrets, or dreams matches a keyword or term in a cell of the attitude/beliefs/summaries table 238, then at operation 738E, table specific search logic 136 will return a response based on the matched cell. For example, table specific search logic 136 may return “my general attituded towards [object of dreams/regrets/hope] is [cell's content] and I hope things get better.”

If the object of User A's hopes, regrets, or dreams matches a keyword or term in a cell of the needs/personality table 240, then at operation 740E, table specific search logic 136 will return a response based on the matched cell. For example, table specific search logic 136 may return “I do need [object of dreams/regrets/hope] and I hope I can get more.”

If the object of User A's hopes, regrets, or dreams matches a keyword or term in a cell of the personal information table 242, then at operation 742E, table specific search logic 136 will return a response based on the matched cell. For example, table specific search logic 136 may return “I do not have [object of dreams/regrets/hope] but I hope to achieve more of that.”

If there is no match the object of User A's hopes, regrets, or dreams matches a keyword or term in any of User A data tables 116, then at operation 744E, table specific search logic 136 will return a response such as “I hope things get better with [object of dreams/regrets/hope].”

FIG. 7F shows one embodiment of the operations executed by table specific search logic 136 when User B's question and/or statement comprises table-specific activation terms related to personal information table 242. The process starts at operation 720F. Next, at operation 722F, table specific search logic 136 searches for keywords or terms in personal information table 242, and synonyms of keywords or terms in personal information table 242 that match the extracted word from User B's statement that relates to a term of personal information. If there is a keyword, term, or synonym match in personal information table 242, then process continues at operation 730E where table specific search logic 136 determines whether the matched cell is blank. If the content of the matched cell is blank, for example, if User A never submitted information related to the specific cell, then table specific search logic 136 may return a response indicating that User A did not submit information related to User B's question at operation 732E. If the matched cell is not blank, then at operation 734F, table specific search logic 136 returns a response based on the contents of the cell. Returning to operation 722F, if there are no keywords, terms, or synonyms matched in dreams/regrets/hopes table 236, then process continues at operation 724F.

At operation 724F, when no keywords, terms, or synonyms are matched to the extracted word from User B's statement that relates to a term of personal information, the table specific search logic 136 will run an upper level search similar to the upper level search described for operation 754B. For example, if an upper level match is identified, then table specific search logic 136 searches for matching keywords and/or terms in personal information table 242. If a match is located between the upper level word and a keyword and/or term in personal information table 242, then, at operation 734E, table specific search logic 136 may return a response based on the content of the matched cell in personal information table 242. The response may be modified based on the identified upper level match and the content of the matched cell in a way similar to the modified response described at operation 752B when an upper level match is identified in life experience table 230. If an identified upper level word does not match a keyword or term in personal information table 242, the table specific search 136 may search identify a type of the upper level word and search for matches in personal information table 242 based on the type of upper level word following a similar process as described in operation 754B.

Returning to operation 724F, if an upper level word is not identified, or if the identified upper level word does not return a match in personal information table 242, or if a type of the upper level word is not identified, or if the type of the upper level word does not return a match in personal information table 242, then the process continues at operation 726F. Based on the a term of personal information in User B's question and/or statement, table specific search logic 136 searches for lower level words that are a “type” of term of personal information, similar to the process described at operation 756B. If such a lower lever term is identified, table specific search logic searches for keyword and/or term matches in personal information table 242. If a match is located between the lower level word and a keyword and/or term in personal information table 242, then, at operation 734F, table specific search logic 136 may return a modified response based on the content of the matched cell in personal information table 242. If no lower level or upper level matches are identified, then the process continues at operation 732F where table specific search logic 136 returns a response indicating that the personal information User B is referencing is unavailable. For example, table specific search logic may return “I'm sorry, this is personal information, and I am unable to inform you about it.”

### Consequence Logic

FIG. 7G shows one embodiment of the operations executed by consequence logic 142 when User B submits a “why” type question. A “why” type question may generally be a question submitted by User B, in response to an answer or response submitted to User B by digital life system 100, where User B further asks about the answer or response. For example, User B may receive a response from digital life system 100, and in response, ask “why?” Further, conversations between the digital copy of User A and User B may be stored and recorded by consequence logic 142. For example, consequence logic 142 may record who asked a question (based on the user ID of User B) and who responded (based on the user ID of User A). Additionally, digital life system 100 may record a consequence keyword from the question and/or statement (e.g. which keyword or term was extracted and/or matched), which cell in which table was activated, and/or which logic (e.g. extraction keyword logic 134, table specific search logic 136, etc.) was activated.

The embodiment of consequence logic 142 shown in FIG. 7G generally shows the operations performed when such a “why” type question is submitted. As explained above, extraction/keyword logic 134 may identify a “why” type question in User B's question and/or statement at operation 504 of FIG. 5. Further, at operation 506, extraction/keyword logic 134 may activate consequence logic 142. When activated, the process executed by consequence logic 142 begins at operation 720G. Next, at operation 722G, consequence logic 142 determines if one of the User A data tables 116 was activated based on User B's previous question and/or statement, if User B's previous question and/or statement caused the activation of fixed answer table/based dialogue logic, or if User B's previous question and/or statement caused the activation of non-personal question logic. Based on which logic was activated, the process continues as explained below.

If the life experience table 230 was activated, then the process continues at operation 724G. At operation 724G, consequence logic 142 locates the “reason” column in life experience table 230 associated with the cell of life experience table 230 that was activated in the previous response. If the cell is blank, then consequence logic 142 may return a response indicating that User A did not submit information related to User B's question. If the cell is not blank, then consequence logic 142 may return a response based on the contents of the cell.

If the human relations table 232 was activated, then the process continues at operation 726G. At operation 726G, consequence logic 142 locates the “what happened” column in human relations table 232 associated with the cell of human relations table 232 that was activated in the previous response. Further, consequence logic 142 locates User A's feeling towards the person associated with the previously activated cell. For example, User A's feeling may be recorded as a feeling score. Based on the “what happened” column contents, and the feeling score, consequence logic 142 may return a response either indicating that User A either likes or dislikes the referenced person. For example, if the feeling score is +3, consequence logic 142 may return a response such as “I like this person.”

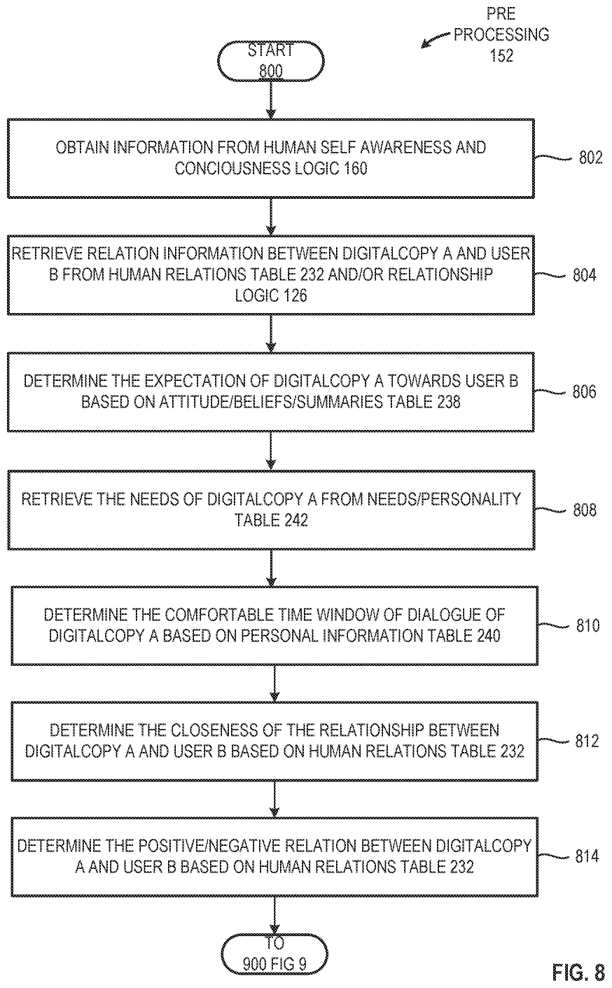

At operation 728G, consequence logic 142 returns a response to User B asking if User B's question was answered. For example, consequence logic 142 may return a response such as “did I answer your question?” If User B responds affirmatively (e.g. “yes”), then the process continues to operation 800 of FIG. 8. If User B responds negatively (e.g. “no”), then the process continues to operation 730G.

At operation 730, consequence logic 142 causes conceptnet.io to search for a “cause” of the previously matched keyword and/or term. Further, consequence logic 142 uses the “causes” identified in concept.io to, and activates interactive table search 140 to search the identified causes by continuing the process at operation 620C of FIG. 6C.

If favorites table 234 was activated, then the process continues at operation 732G. At operation 732G, consequence logic 142 returns a response such as “I just like that.”

If the attitude/beliefs/summaries table 238 was activated, then the process continues at operation 734G. At operation 734G, consequence logic 142 retruns a response such as “the reason is complicated and mainly due to my personal experience.” The process then continues at operation 730G where “causes” of the previously matched keywords and/or term are searched using concept.io.

If personal information table 240 was activated, then the process continues at operation 736G. At operation 732G, consequence logic 142 returns a response such as “no specific reason, it is a result of either genetics or environment.”

If fixed answer table/basic dialogue logic was activated, then the process continues at operation 738G. At operation 738G, consequence logic 142 locates the “reason” column in fixed answer table associated with the cell of fixed answer table that was activated in the previous response, then consequence logic 142 may return a response based on the contents of the cell.

Finally, if non-personal question logic was activated, then the process continues at operation 740G. At operation 740G, consequence logic 142 may return a response such as “please google the reason for this answer.”

### Active Talking Logic

Returning to FIG. 1, the exemplary embodiment of the digital life system 100 is shown as comprising active talking logic 150. As a second user (for example, User B) interacts with digital life system 100, the active talking logic of FIG. 1 performs operations related to generating questions and/or statements directed towards the second user (for example, User B) based, in part, on information collected about a first user (for example, User A). Further, these questions and/or statements may be generated without first receiving a question and/or statement from User B. In addition, active talking logic 150 may monitor timing related aspects of a conversation between User B and the digital copy of User A and generate questions and/or statements based on the timing of the conversation. For example, active talking logic 150 may track how long User B has been talking to the digital copy of User A, how long User B has been silent, the time of the day, and the digital copy of User A's comfortable window of talking. Thus, based on these timing-related aspects of a conversation between User B and the digital copy of

User A, active talking logic 150 is beneficially able to initiate a conversation with User B based on multiple, specific parameters that a human in a conversation may not otherwise be able to monitory. Further, active talking logic 150 is beneficially able to generate questions and/or statements based on User A data tables 116 which comprise information that may not otherwise be stored or readily recalled by the human mind. Moreover, the topics that each question and/or statement is related to may be chosen based on specific probabilities, thus active talking logic 150 is beneficially able to initiate conversations that may otherwise be more diverse that conversations generated by the human mind. As show in the exemplary embodiment of digital life system 100 in FIG. 1, active talking logic 150 comprises pre-processing 152, time management logic 154, active dialogue logic 156, and knowledge update logic 158.

### Preprocessing

Preprocessing 132 embodied in FIG. 1 generally performs operations related to retrieving information about User A and User B to prepare for the activation of active dialogue logic 156. Specifically, preprocessing 152 may perform operations related to retrieving self-awareness information from human self-awareness and consciousness logic 160, determining the relationship between User A and User B, determining User A's expectations of User B, retrieving the needs of User A, determining the comfortable time window of dialogue of the digital copy of User A, determining the closeness of User A's relation to User B, and determining how positive or negative the relationship between User A and User B is.

FIG. 8 shows one embodiment of preprocessing 152. In this example, the operations performed by preprocessing 152 begin at operation 800. Next, at operation 802, preprocessing 152 retrieves information form human self-awareness and consciousness logic 160. The process then continues at operation 804 where preprocessing 152 retrieves relationship information between User A and User B. For example, the relationship information may be retrieved based on information stored in human relations table 232 and/or based on relationship logic 126.

At operation 806, preprocessing 152 determines the expectation of User A and/or the digital copy of User A towards User B. For example, the expectation towards User B may be based on information stored in attitude/beliefs/summaries table 238. Preprocessing 152 determines a population group to which User B belongs based on the relationship information between User A and User B. Then, based on User B's population group, and based on User A's attitude and/or belif towards that population group from information stored in attitude/beliefs/summaries table 238, preprocessing 152 determines User A's expectation towards User B. If preprocessing 152 is unable to locate User A's attitude and/or belief towards that population group from information stored in attitude/beliefs/summaries table 238, then using a pre-prepared table (e.g. non-personal table), preprocessing 152 determines and estimated expectation of User A towards User B. In one embodiment, the pre-prepared table may comprise general expectations based on relationships. For example, the pre-prepared table may indicate that a father's expectation towards his son usually involves growth of knowledge, health, maturity, etc.

At operation 808, preprocessing 152 retrieves the needs of User A and/or of the digital copy of User A. For example, the needs of User A may be based on information stored in needs/personality table 242.

At operation 810, preprocessing 152 determines the comfortable time window of dialogue of the digital copy of User A. For example, personal information table 240 may contain information in response to the question “are you an early bird, a night owl, or do you not follow a pattern?” Based on the information provided by User A in response to this question, the comfortable time window of dialogue may be determined. In one embodiment, an early bird's comfortable time window of dialogue may be between 6 am to 10 pm and a night owl's comfortable time window of dialogue may be between 8 am to 2 am.

At operation 812, preprocessing determines the closeness of the relationship between User B and User A (and/or the digital copy of User A). For example, the closeness of the relationship may be determined based on the relationship information retrieved at operation 804. In one embodiment, if User A and User B are members of the same nuclear family, then the relationship between User A and User B is close. If User A and User B are relatives or friends, and if the number of words in the row of human relations table 232 related to User B has more than 40 words, then the relationship between User A and User B is close. No matter what the relationship between User A and User B, if the number of words in the row of human relations table 232 related to User B has more than 60 words, then the relationship between User A and User B is close. If none of the above conditions are satisfied, then the relationship between User A and User B is not close.

At operation 814, preprocessing determines if the relationship between User B and User A (and/or the digital copy of User A) is positive or negative. For example, whether the relationship between User A and User B is positive or negative may be determined based on humans relations table 232. In one embodiment, human relations table 232 has information related to an evaluation score of User A towards User B. If the evaluation score is negative, then the relationship is negative. If the evaluation score is positive, then the relationship is negative. If the score is zero, the relationship may be positive. Following the completion of 814, time management logic 154 is activated and the process continues at operation 900.

### Time Management Logic

Time management logic 154 embodied in FIG. 1 generally performs operations related to determining when to generation and submit questions and/or statements to a user interacting with digital life system 100 (for example, User B) based on timing-related aspects of a conversation between the user (User B) and a digital copy of a second user (User A). For example, timing-related aspects of the conversation between User B and a digital copy of User A may be when the dialogue between User B and the digital copy of User A starts, the current time for User B, the comfortable window of dialogue for User A, and the silence time between a question and/or statement submitted to User B and User B's next statement and/or question.

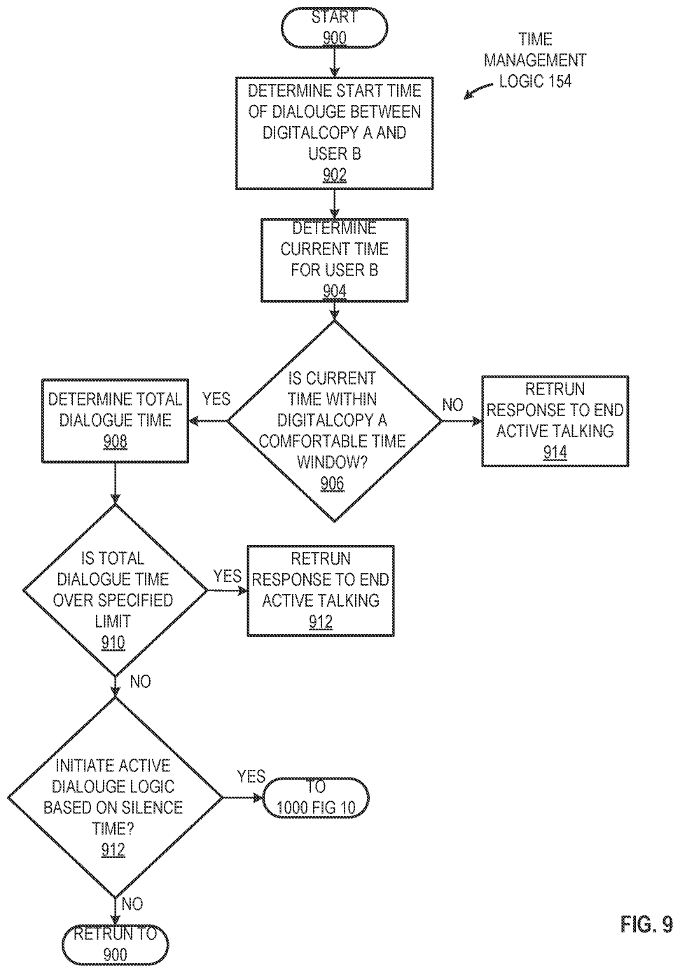

FIG. 9 shows one embodiment of the operations performed by time management logic 154. In this example, the operations performed by time management logic 154 begin at operation 900. Next, at operation 902, time management logic 154 determines the time when dialogue between User B and the digital copy of User A starts. For example, the start time may be defined by a time of day such as 8:30 pm. Next, at operation 904, time management logic 154 retrieves the current time for User B. Again, the current time may be defined by a time of day such as 9:15 pm. Next, at operation 904, time management logic 154 determines if the current time is within the comfortable window of dialogue for the digital copy of User A. If the current time is outside of the comfortable time window, and if User A is classified as either an early bird or a night owl, then the process continues to operation 914 and time management logic 154 returns a response such as “You know I am [a night owl/early bird] and it is [current time] right now. I hope to get some rest, let's talk next time.” If the current time is within the comfortable window of dialogue, then the process continues at operation 908.

At operation 908, time management logic 154 determines the total dialogue time of the conversation between User B and the digital copy of User A and the process continues to operation 910. Next, at operation 910, time management logic 154 determines if the total dialogue time of the conversation between User B and the digital copy of User A is over a prespecified limit based on the digital copy of User A's age. In one embodiment, if the age of the digital copy of User A is over 60 and the total time of dialogue is over 30 minutes, then the process continues to operation 912 and time management logic 154 a response such as “We have talked over 40 min, I am tired, can we talk next time?” Similarly, if the age of the digital copy of User A is equal to or less than 60 and the total time of dialogue is over 60 minutes, then the process similarly continues to operation 912 and time management logic 154 a response such as “We have talked over 70 min, I am tired, can we talk next time?” If the total time of dialogue is within the prespecified limit, then the process continues to operation 912.

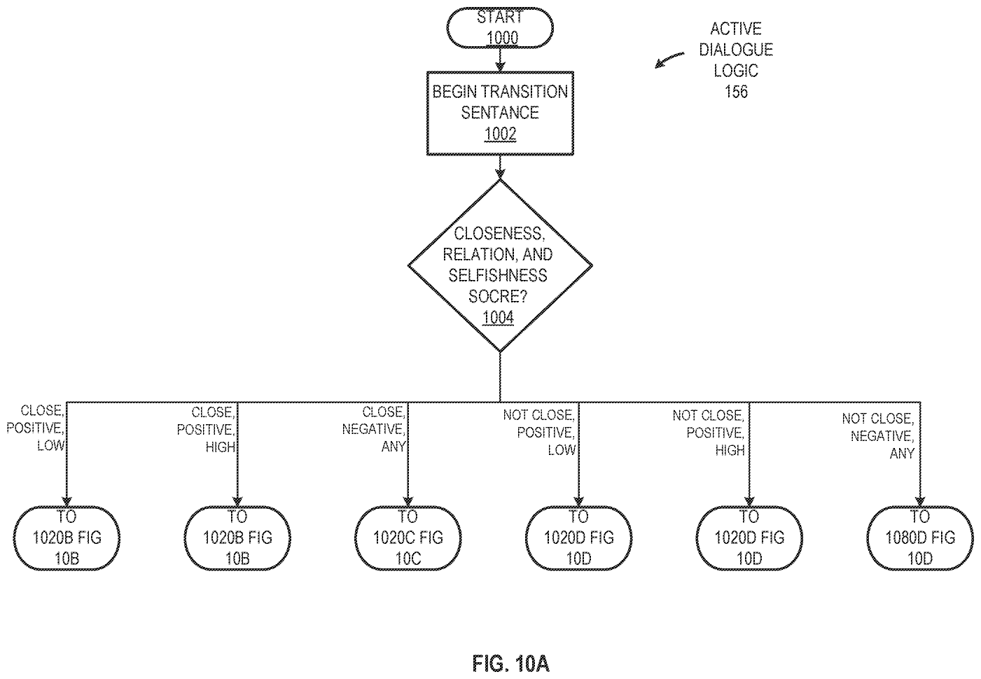

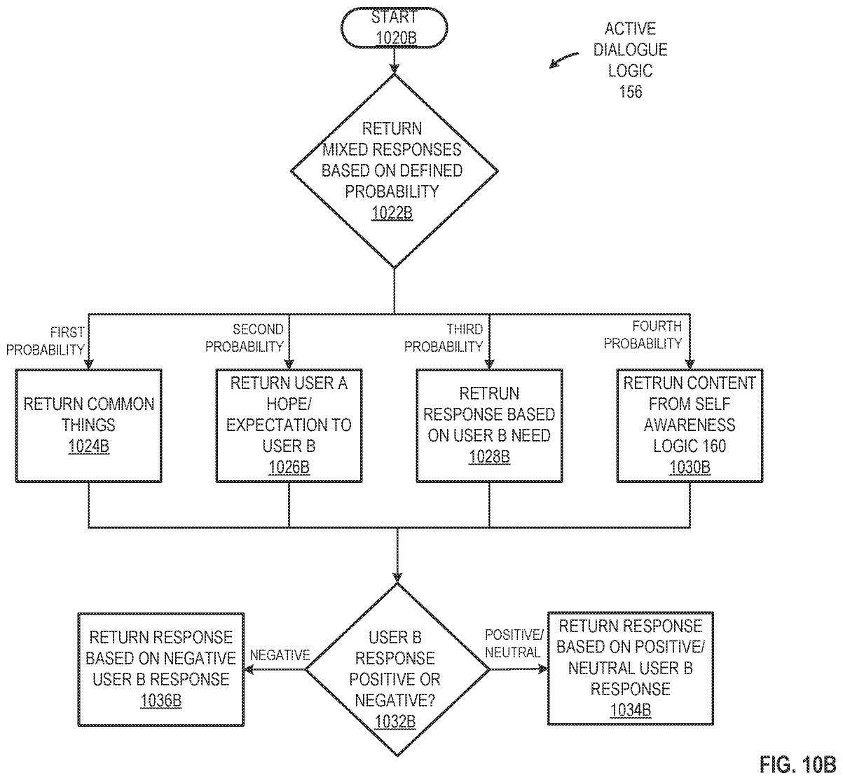

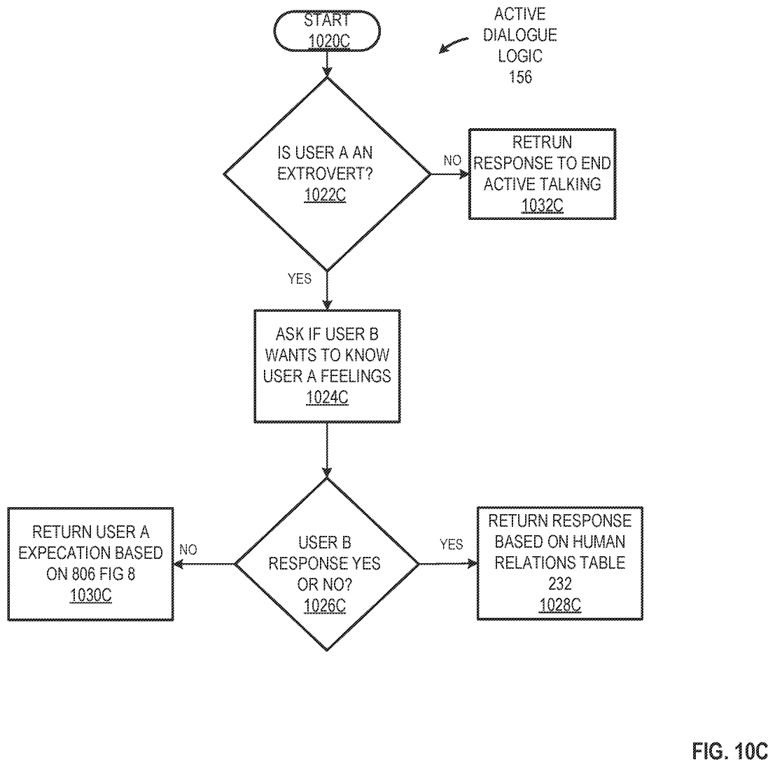

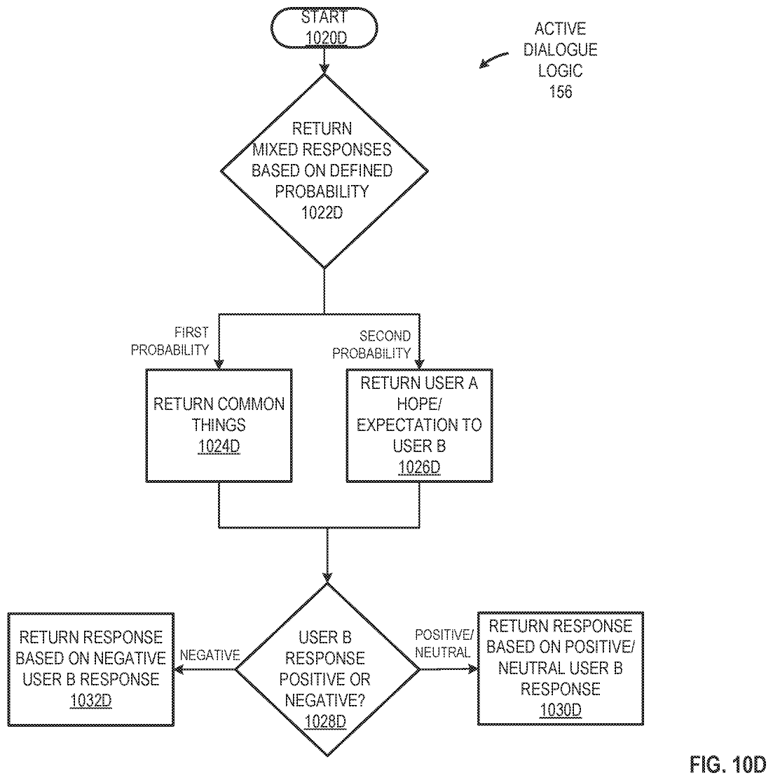

At operation 912, time management logic 154 may determine whether to active dialogue logic 156 based on the silence time between a first statement/question from User B and a second statement/question from User B. In one embodiment, if User A is classified as an extrovert and a type-A person, then time management logic 154 may activate active dialogue logic 156 if the time of silence is greater than 3 seconds. If User A is classified as an extrovert and but not a type-A person, then time management logic 154 may activate active dialogue logic 156 if the time of silence is greater than 5 seconds. If User A is classified as an introvert and a type-A person, then time management logic 154 may activate active dialogue logic 156 if the time of silence is greater than 3 seconds. Finally, if User A is classified as an introvert and not a type-A person, then time management logic 154 may activate active dialogue logic 156 if the time of silence is greater than 6 seconds. In any of the above-mentioned scenarios, when the active dialogue logic 156 is activated, the process continues to operation 1000 of FIG. 10.

### Active Dialogue Logic

Active dialogue logic 156 embodied in FIG. 1 generally performs operations related to generating questions and/or statements to submit to User B based on the information stored in User A data tables 116. Further, these questions and/or statements may be generated without a prior question and/or statement from User B. Moreover, the generation of questions and/or statements may be based on the closeness of the relationship between User A and User B as determined by preprocessing 152 at operation 812, whether User A and User B's relationship is positive or negative as determined by preprocessing 152 at operation 814, and the selfishness score of User A.

FIG. 10A shows one embodiment of the operations performed by active dialogue logic 156. In this example, the operations performed by active dialogue logic 156 begin at operation 1000. Next, at operation 1002, active dialogue logic 156 generates a transition sentence to submit to User B. For example, the transition sentence generated may be “I hope/want to say/talk/speak something . . . ” The process then continues at operation 1004.

At operation 1004, active dialogue logic 156 determines which type of question and/or statement to submit to User B based on the closeness of the relationship between User A and User B as determined by preprocessing 152 at operation 812, whether User A and User B's relationship is positive or negative as determined by preprocessing 152 at operation 814, and the selfishness score of User A. If User A and User B have a close, positive relationship and User A has a low selfishness score, then the process continues to operation 1020B of FIG. 10B. If User A and User B have a close, positive relationship and User A has a high selfishness score, then the process continues to operation 1020B of FIG. 10B. If User A and User B have a close, negative relationship and User A has any selfishness score, then the process continues to operation 1020C of FIG. 10C. If User A and User B have a not close, positive relationship and User A has a low selfishness score, then the process continues to operation 1020D of FIG. 10D. If User A and User B have a not close, positive relationship and User A has a high selfishness score, then the process continues to operation 1020D of FIG. 10D. Finally, if User A and User B have a not close, negative relationship and User A has a any selfishness score, then the process continues to operation 1020D of FIG. 10D.

FIG. 10B shows one embodiment of the operations performed by active dialogue logic 156 when User A and User B have a close, positive relationship and User A has a either a high or low selfishness score. In this example, the operations performed by active dialogue logic 156 begin at operation 1020B. Next, at operation 1022B, active dialogue logic 156 returns mixed responses of questions and/or statements based on predefined probability. The responses returned may be related to common things, User A's expectation or hope towards User B, User B's needs if User B has submitted information to the needs/personally 242 for User B's profile, and content from human self-awareness and consciousness logic 160. Responses returned related to common things may be, for example, questions asking how the weather is at User B's location, who User B's job is going, etc. Responses returned related to User B's needs may be, for example, statements like “I know you need . . . ,” “I hope you are lucky to get that, but you should try your best,” etc.

In one embodiment, when User A and User B have a close, positive relationship and User A has a low selfishness score, then active dialogue logic 156 may return responses related to common things with a 20% probity at operation 1024B, responses related to User A's expectation or hope towards User B with a 20% probability at operation 1026B, responses related to User B's needs with a 20% probability at operation 1028B, and content from human self-awareness with a 20% probability at operation 1030B. Similarly, when User A and User B have a close, positive relationship and User A has a high selfishness score, then active dialogue logic 156 may return responses related to common things with a 13.3% probity at operation 1024B, responses related to User A's expectation or hope towards User B with a 13.3% probability at operation 1026B, responses related to User B's needs with a 13.3% probability at operation 1028B, and content from human self-awareness with a 60% probability at operation 1030B.

At operation 1032B, for each of the above-mentioned responses submitted to User B, active dialogue logic 156 may determine whether User B's reply is positive, neutral, or negative. If User B's reply is positive or natural, then active dialogue logic 156 may return an additional response such as “Ok/yep/fine, I am happy/glad to talk with you” at operation 1034B. Conversely, if User B's reply is negative, then active dialogue logic 156 may return an additional response such as “It looks you are not happy or something is wrong, let me think/suggest discuss with a real person/maybe your other friends or relatives can help you” at operation 1036B.

FIG. 10C shows one embodiment of the operations performed by active dialogue logic 156 when User A and User B have a close, negative relationship and User A has a either any selfishness score. In this example, the operations performed by active dialogue logic 156 begin at operation 1020C. Next, at operation 1022C, active dialogue logic 156 determines if User A is an extrovert type person. If User A is not an extrovert (an introvert) then active dialogue logic 156 will return a response after a delay such as “I hope to not speak with you at this time” or another similar statement at operation 1032C. The delay may, for example, be 2-3 seconds. Conversely, if User a is an extrovert, then the process continues at operation 1024C.

At operation 1024C, active dialogue logic 156 will submit a response asking User B if User B wants to know User A's true feeling towards User B. For example, active dialogue logic 156 may submit a response such as “do you want to hear my true feelings towards you? They may not be positive.” Next, at operation 1026, based on User B's reply, active dialogue logic 156 determines if User be replied affirmatively (“yes”) or negatively (“no”). If User B's reply was affirmative, then the process continues at operation 1028C and active dialogue logic 156 returns a response based on User A's feelings towards User B′ from human relations table 232. If User B replies further, then active dialogue logic 156 may return the whole row related to User B in human's relation table 232. Conversely, if User B's reply was negative, then the process continues at operation 1030C and active dialogue logic 156 returns a response based on User A's expectation towards User B′ from operation 806 of FIG. 8 of preprocessing 152.

FIG. 10D shows one embodiment of the operations performed by active dialogue logic 156 when User A and User B have a not close, positive or negative relationship and User A has a high, low, or any selfishness score. In this example, the operations performed by active dialogue logic 156 begin at operation 1020D. Next, at operation 1022D, active dialogue logic 156 returns mixed responses of questions and/or statements based on predefined probability. The responses returned may be related to common things, related to User A's expectation or hope towards User B, or indicating that User A no longer currently wishes to speak to User B. Responses returned related to common things may be, for example, questions asking how the weather is at User B's location, who User B's job is going, etc.

In one embodiment, when User A and User B have a not close, positive relationship and User A has a low selfishness score, then active dialogue logic 156 may return responses related to common things with a 50% probity at operation 1024D, and responses related to User A's expectation or hope towards User B with a 50% probability at operation 1026D. Similarly, when User A and User B have a not close, positive relationship and User A has a high selfishness score, then active dialogue logic 156 may return responses related to common things with a 70% probity at operation 1024D, responses related to User A's expectation or hope towards User B with a 30% probability at operation 1026D. Finally, when User A and User B have a not close, negative relationship and User A has a any selfishness score, then active dialogue logic 156 may return responses related to common things with a 30% probity at operation 1024D, responses indicating that User A no longer currently wishes to speak to User B with a 70% probability at operation 1026D. A response indicating that User A no longer currently wishes to speak to User B may be, for example “I hope to not speak with you at this time” or similar statements.

At operation 1028D, for each of the above-mentioned responses submitted to User B, with the exception of responses indicating that User A no longer currently wishes to speak to User B, active dialogue logic 156 may determine whether User B's reply is positive, neutral, or negative. If User B's reply is positive or natural, then active dialogue logic 156 may return an additional response such as “Ok/yep/fine, I am happy/glad to talk with you” at operation 1030D. Conversely, if User B's reply is negative, then active dialogue logic 156 may return an additional response such as “It looks you are not happy or something is wrong, let me think/suggest discuss with a real person/maybe your other friends or relatives can help you” at operation 1032D. If User B replies to responses indicating that User A no longer currently wishes to speak to User B, then active dialogue logic 156 may return the whole row related to User B in human relations table 232.

### Knowledge Update Logic

Knowledge update logic 158 embodied in FIG. 1 generally performs operations related to storing and classifying information based on User B's replies to questions and/or statements submitted by active dialogue logic 156. Specifically, knowledge update logic 158 may determine if a reply from User B contains a new relation word that is not already in human relations table 232 and respond based on that new relation word. For example, if User B's reply contains a new relation word, knowledge update logic 158 may return “I am sorry that I don't have such a person in my pre-prepared memory; However, I am happy to learn that person if you can answer the following questions.” Then, knowledge update logic 158 will prompt User B to answer questions as described with respect to human relations 212 of User A inputs 112. Further, knowledge update logic 158 and may determine if User B's reply is positive or negative and return responses based on how many positive and/or negative responses User B has submitted. Additionally, knowledge update logic 158 may update User A and User B's relationship classification from positive or neutral to negative based on User B's replies.

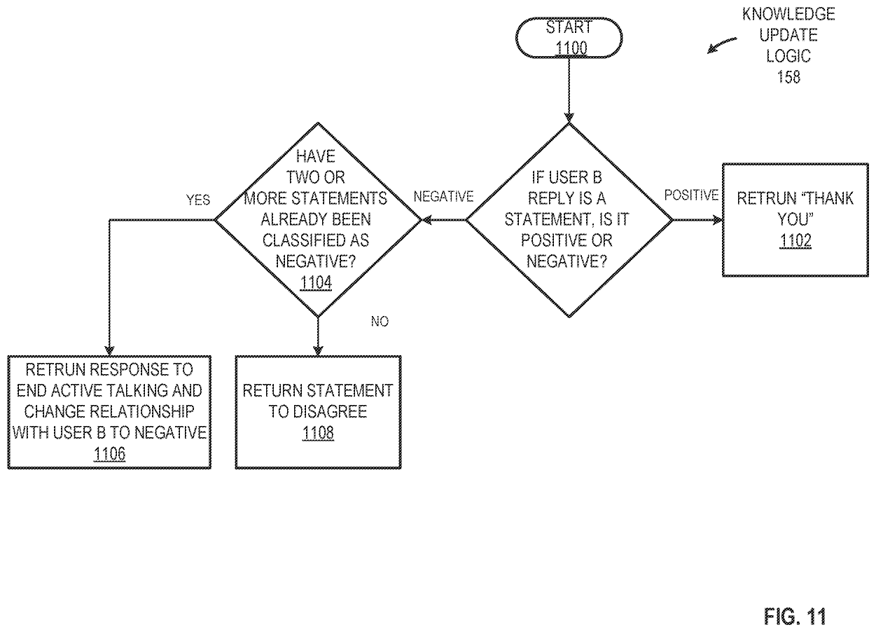

FIG. 11 shows one embodiment of the operations performed by knowledge update logic 158. In this example, the operations performed by knowledge update logic 158 begin at operation 1100. Next, at operation 1102, knowledge update logic 158 classifies User B's reply as positive or negative. In one embodiment, User B's reply must be a statement rather than a question. Further, the statement may be classified as positive or negative based on a pre-prepared database. If the statement is classified as positive, then, at operation 1104 knowledge update logic 158 may return a response such as “thank you.” If the statement is classified as negative, then the process continues at operation 1106. At operation 1106, knowledge update logic 158 determines how many statements from User B have previously been classified as negative. If more than two statements have already been classified as negative, then the process continues at operation 1108 and knowledge update logic 158 returns a statement such as “Sorry, I don't hope to talk with you any longer. You don't respect others.” Further, knowledge update logic 158 may change the relationship classification between User A and User B to negative. If more than two or fewer statements have been classified as negative, then the process continues at operation 1106 and knowledge update logic 158 returns a statement such as “Sorry. I do not accept that since everyone has something good.”

In one embodiment statements may be classified as positive or negative based on the following criteria. For example, if User B's statement contains only “You” or “Your” (with no other pronouns), negative words (such as no, not, dis-, un- etc.), and a negative adjective (e.g., stupid) or a negative noun (e.g., fool), then it will be classified as positive. Similarly, if User B's statement contains only “You” or “Your” (with no other pronouns), no negative words (such as no, not, dis-, un- etc.), and a positive adjective (e.g., smart) or a negative noun (e.g., wisdom), then it will be classified as positive. Conversely, if User B's statement contains only “You” or “Your” (with no other pronouns), no negative words (such as no, not, dis-, un- etc.), and a negative adjective (e.g., stupid) or a negative noun (e.g., fool), then it will be classified as negative. Similarly, if User B's statement contains only “You” or “Your” (with no other pronouns), negative words (such as no, not, dis-, un- etc.), and a positive adjective (e.g., smart) or a negative noun (e.g., wisdom), then it will be classified as negative.

### Human Self Awareness and Consciousness Logic

Returning to FIG. 1, the exemplary embodiment of the digital life system 100 is shown as comprising human self-awareness and consciousness logic 160. Human self-awareness and consciousness logic 160 generally records, models, generates and causes visualization a digital copy of a user's (User A's) individual self-awareness, consciousness, sensation, and feeling towards that digital copy's body, time, date, and different natural, human-made, and social environments. Thus, human self-awareness and consciousness logic 160 is beneficially able to capture the environment and surroundings and generate a self-awareness and consciousness for a digital copy of a User. As depicted by the embodiment in FIG. 1, human self-awareness and consciousness logic 160 comprises time dimension logic 164, body dimension logic 168, space dimension logic 172, and integrated dimensions logic 174.

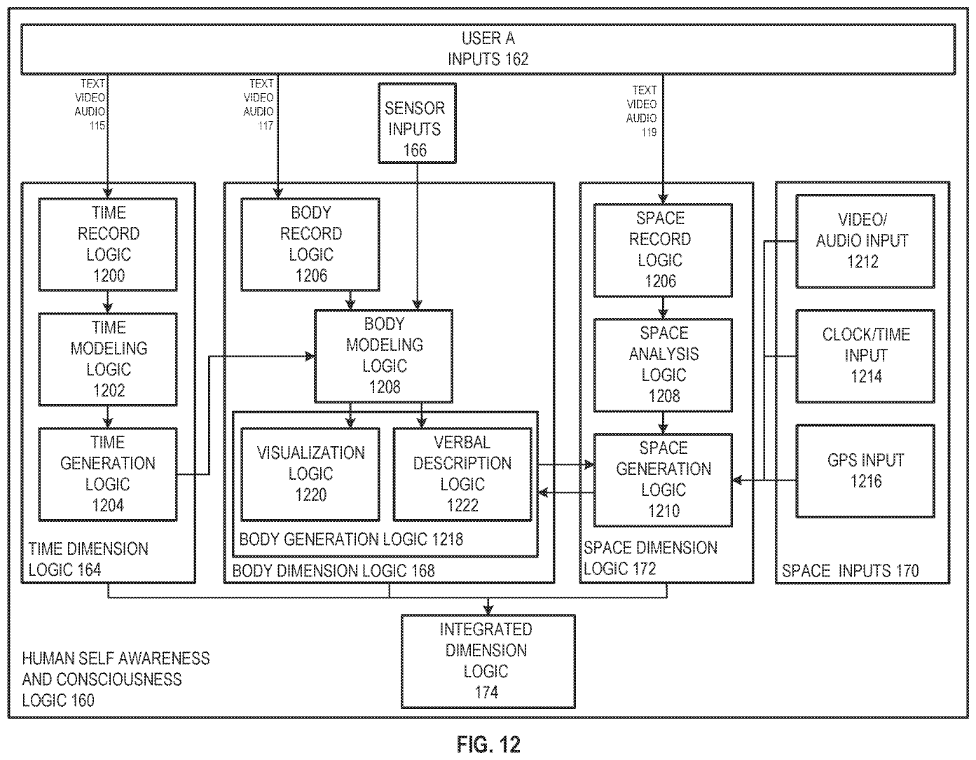

FIG. 12 shows one embodiment of human self-awareness and consciousness logic 160. Time dimension logic 164 generally records, models, and generates an individual self-awareness related to biological clock, time and date for the digital copy of User A. The time dimension logic 164 of FIG. 12 comprises time record logic 1200, time modeling logic 1202, and time generation logic 1204. First, time record logic 1200 records User A's biological clock and energy level. For example, time record logic 1200 may prompt User A to answer questions related to User A's typical biological clock cycle and energy levels throughout a typical day. FIG. 12 shows the inputs from User A in response to these questions as User A inputs 162. User A inputs may be capture as may be captured by digital life system 100 in the form of text, audio, and/or video. In one embodiment, time record logic 1200 prompts User A to answer questions such as: normal wake up time, normal breakfast time (or no breakfast), normal lunch time (or no lunch), do you normally take a nap after lunch (and how long is it), normal dinner time, normal sleep time, after how many hours do you usually get tired after continuously working, and are you an energetic person. Further, time record logic 1200 may prompt User A ask to answer questions related to their individual human's feeling/awareness towards of holiday and birthdays such as: “Please list 1-10 most important dates (e.g., holiday, birthday etc.) in a year for you, and your feeling/awareness of each of these dates.”

Time modeling logic 1202 generally models User A's individual human biological clock which may be reflected with statuses such as hungry, sleepy and fatigue levels. For example, based on User A inputs 162 provided to time record logic 1200 (e.g. WakeupT (0-24 format), BreakfastT, LunchT, NapT, DinnerT, SleepT, TiredT, EnergyPerson (range: 1-4)), time modeling logic 1202 models the digital copy of User A's individual human hungry level (Hungryt,i), sleepy level (Sleept,i), and fatigue level (Fatiguet,i) at current time (t) (0-24 format). For example, in one embodiment, hungry level may be modeled by the following equation:

Hungryt, i=EnergyPerson×(t−Max(Breakf sastT, LunchT, DinnerT))

For example, if it is 13:00, if User A with EnergyPerson=1 may have just had lunch at noon (12). In that case, User A's hungry level of that person will be, for example 1×(13−Max(7, 12, unknown))=1×(13−12)=1. Similarly, sleepy level may be modeled by the following equation;

$$Sleept , i = Max ( \frac{1}{EnergyPerson} ⨯ \vert \frac{1}{t - SleepT} \vert , \frac{1}{EnergyPerson} ⨯ \vert \frac{1}{t - NapT} \vert )$$

For example, if it is 21:00, User A's EnergyPerson=1 if User A′ normally sleeps around 22:00 (no nap after lunch). Then the sleepy level of User A will be Sleept,i=(1/1)×|1/(21-22) |=1. In comparison, if it is 21:45, and User A's EnergyPerson=1 because User A normally sleep around 22:00, then the sleepy level of that person will be Sleept,i=(1/1)×|1/(21.75-22)|=4. Similarly, fatigue level may be modeled by the following equation:

$$Fatigue , i = \frac{1}{EnergyPerson} ⨯ \frac{1}{TiredT} ⨯ Ttask ⨯ Sleept , i ⨯ \\ ( MenstrualPeriod , female only )$$

Where Ttask is the length of time the digital copy of User A is engaged in certain tasks (e.g., a conversation). When the digital copy of User A experiences a longer conversation, its fatigue level will increase. MenstrualPeriod(female only) defines the effect of menstrual period on female's fatigue level. It is a non-linear decline function given: days in a menstrual cycle=[current date-starting date of a regular menstrual cycle of female, if current date>=starting date and days in a menstrual cycle<=15], the current date, and the female's age. For example:

$$MenstrualPeriod = \frac{1}{days in a menstrual cycle ( user defined )}$$

Time generation logic 1202 generally generates verbal expressions and visual display related to biological clock and time perception. First, time generation logic 1202 it retrieves the value of the variables from the User A as user a inputs 162 (e.g., BreakfastT, LunchT, NapT, DinnerT, SleepT, TiredT, EnergyPerson), then it compares the current time (t) when digital life system 100 is in usage (e.g., during a conversation with a questioner, or when the system is running by itself) and length of conversation (Ttask). All of these values are entered into the equations above by time generation logic 1202. After solving the equations, time generation logic 1202 generate verbal expressions and causes visual display if hungry level (Hungryt,i), sleepy level (Sleept,i), or fatigue level (Fatiguet,i) reach certain thresholds. For example, if Fatiguet,i is too high, the time generation logic 1202 will generate verbal expression such as “I feel a bit tired in continuing our conversation, can we talk next time?” Further, time generation logic 1202 may cause display/audio logic 1306 to display a red icon over a an image of the digital copy of User A's stomach area to indicate the hungry level. Display/audio logic 1306 may also display sleepy icon (e.g. ZZZ) and the animation of posture (e.g., head nodding and longer closure of eyes) of the the digital copy of User A to indicate the sleepy level and fatigue level.

Body Dimension Logic 168 embodied in FIG. 12 generally records, models, and generates an individual self-awareness related to the human body for the digital copy of User A by recording, organizing and visualizing an individual human's whole body sensation. The body dimension logic 168 of FIG. 12 comprises body record logic 1206, body modeling logic 1208, and body generation logic 1218. Further body generation logic 1218 may comprise visualization logic 1210 and verbal description logic 1204. Body record logic 1206 may prompt an individual human to use a graphic interface (implemented in APP/Webpage etc.) to indicate certain regions of their body or their whole body's sensations in terms of triggering sources (disease/injuries, desire, external environment, etc.), feeling itself (pain, discomfort, desire/arousal, emotion (e.g., pleasant), itchy, etc.), time and frequency of these sensations (all the time, hourly, day/night, etc.), and degree of strength (1: Very light to 10: Strongest). As User A makes selections, the selections are provided as User A inputs 162. For example, if User A has experienced rheumatic pain at his left knee, User A can put an icon on that location using the graphic interface. Further, body dimension logic 168 may prompt User A to indicate trigger sources, feeling itself, time and frequency, and degree of strength. In addition, body dimension logic 168 may ask User A for his/her: 1) comfort range of environmental temperature, body motion movement (acceleration, deceleration, rotation), noise, brightness, air-pressure, vibration, gustatory perception, and olfactory perception, and 2) the weight (Wi) of his/her body parts in determining his/her overall (whole) body sensation.

Body modeling logic 1208 generally models components of human body parts' sensations. For example, Based on the self-reported data from the body dimension logic 168, the body modeling logic 1208 will adjust the value of a parameter Ai (A1, A2, A3, etc. for different body parts) according to the following function: Sensation of Body Part i (Content, Location, Strength, Length and Frequency)=Ai(Individual self-entry, sensor's input from the current environment, fatigue/hungry/sleepy level, current time). For example, if the environment's temperature is out of the comfort range of User A, the level of discomfort for the digital copy of User A will increase. body modeling logic 1208 also models the digital copy of User A's overall feeling of the digital copy's own body by integrating all of sensations from different body parts together by the following equation:

Overall Sensation=Σ(Wi×Sensatoin of Body Part i)

Where Wi is the weight of a body part in affecting the overall sensation which came from the self-report by body dimension logic 168. In addition, based on the body movement from different body parts, the body modeling logic 1208 classifies the body movement into: walking, staying still in one location, in a car/airplane/bike etc., indicating the status of the body for “what I am doing.” Further, body modeling logic 1208 may receive input from time generation logic 1204 and space dimension logic 172.

Body generation logic 1218 generally generates body parts sensations by causing visualization logic 1220 and verbal description logic 1222 to cause the display or verbalization of the digital copy of User A's sensations. Body generation logic 1218 may receive inputs from sensor inputs 166, which may comprise various sensors, and clock to determine the time of the external world. Embodiments of body generation logic 1218 and body dimension logic 168 may be implemented in a complex version or as a smartphone version.

Visualization logic 1220 may integrate all of the body parts self-entry data and causes the display of 3D graphics representing the digital copy of User A on display/audio logic 1306. For example, visualization logic 1220 may cause the display of different colors to represent feeling of the whole body and body parts (e.g., red indicates pain or discomfort, blue indicates comfort etc., the density of dots will be correlated with the strength of the feeling/sensation). For example, visualization logic 1220 may cause the display of the digital copy of User A with red colored dots to indicate an User A's pain on left knee under wet/cold environment.

The table 1 and table 2 below specifie the color, density and transparency level of colored dots, animations of color dots and emotion of face, and their meanings in the visualization and design as implemented by and exemplary embodiment of visualization logic 1220.

TABLE 1

| Color | Meaning |
|---|---|
| Red | Negative Feelings (e.g., Pain, sore, swollen etc.) |
| Light blue | Positive Feelings (e.g., Pleasant, comfortable |
| | enjoyment) |

TABLE 2

| | | | Animation of | |
|---|---|---|---|---|
| | | | Emotion of the | |
| | Density and | Animation of | Human Face | |
| Color | transparency | Body Parts | and Gesture | Meaning |
| Red | High, 0-20% | High frequency | High frequent | Frequent and |
| | transparency | blink rate (e.g. over | strong | Sharp and strong |
| | | 3 times per sec) | unpleasant | negative feeling |
| | | | emotion on the | |
| | | | face | |
| Red | High, 0-20% | Low frequency | Low frequent | Low frequency |
| | transparency | blink rate (e.g., 1 | strong | pain but each time |
| | | time per sec or low) | unpleasant | is strong negative |
| | | | emotion on the | feeling |
| | | | face | |
| Red | Medium, 40- | High frequency | High frequent | Frequent and |
| | 60% | blink rate (e.g. over | medium | medium level of |
| | transparency | 3 times per sec) | unpleasant | negative feeling |
| | | | emotion on the | |
| | | | face | |
| Red | Medium, 40- | Low frequency | Low frequent | Low frequency |
| | 60% | blink rate (e.g., 1 | medium | pain but each time |
| | transparency | time per sec or low) | unpleasant | is medium level of |
| | | | emotion on the | negative feeling |
| | | | face | |
| Red | Low, 80-90% | High frequency | High frequent | Frequent and low |
| | transparency | blink rate (e.g. over | slight unpleasant | level of negative |
| | | 3 times per sec) | emotion on the | feeling |
| | | | face | |
| Red | Low, 80-90% | Low frequency | Low frequent | Low frequency |
| | transparency | blink rate (e.g., 1 | slight unpleasant | pain but each time |
| | | time per sec or low) | emotion on the | is low level of |
| | | | face | negative feeling |
| Light | High, 0-20% | High frequency | High frequent | Frequent and |
| Blue | transparency | blink rate (e.g. over | strong pleasant | strong positive |
| | | 3 times per sec) | emotion on the | feeling |
| | | | face | |
| Light | High, 0-20% | Low frequency | Low frequent | Low frequency |
| Blue | transparency | blink rate (e.g., 1 | strong pleasant | pain but each time |
| | | time per sec or low) | emotion on the | is strong positive |
| | | | face | feeling |
| Light | Medium, 40- | High frequency | High frequent | Frequent and |
| Blue | 60% | blink rate (e.g. over | medium pleasant | medium level of |
| | transparency | 3 times per sec) | emotion on the | positive feeling |
| | | | face | |
| Light | Medium, 40- | Low frequency | Low frequent | Low frequency |
| Blue | 60% | blink rate (e.g., 1 | medium pleasant | pain but each time |
| | transparency | time per sec or low) | emotion on the | is medium level of |
| | | | face | positive feeling |
| Light | Low, 80-90% | High frequency | High frequent | Frequent and low |
| Blue | transparency | blink rate (e.g. over | slight pleasant | level of positive |
| | | 3 times per sec) | emotion on the | feeling |
| | | | face | |
| Light | Low, 80-90% | Low frequency | Low frequent | Low frequency |
| Blue | transparency | blink rate (e.g., 1 | slight pleasant | pain but each time |
| | | time per sec or low) | emotion on the | is low level of |
| | | | face | positive feeling |

Verbal description logic 1222 may will also generate a verbal description of the digital copy of User A's sensation based on body modeling logic 1208. The verbal description generated by verbal description logic 1222 may be composed of: “My whole body/body part+feels/sees/hears+sensation degree (e.g., strong)+sensation content (pain, discomfort, pleasant, arousal, etc.).” In addition, when verbal description logic 1222 may communicate with inputs from a video camera, a microphone and/or face recognition logic, to enable the recognition of a user's voice and face.

Space dimension logic 172 embodied in FIG. 12 generally records, models, and generates an individual self-awareness related to the inputs related to the digital copy of User natural, human-made, and social environment. The space dimension logic 172 of FIG. 12 comprises space record logic 1206, space analysis logic 1208, and space generation logic 1210. Space record logic 1206 prompts User A to provide User A's sensation/feeling given different categories of natural, man-made, and social environments. First, Table 3 below may be used by space record logic to cause display/audio logic 1306 to present the stimuli (image or videos) to User A and collect User A's sensation/feelings/and self-awareness as User A inputs 162. Second, a graphic UI interface may be presented to the User A prompt User A to input sensation/feelings/and self-awareness and the degree of likeness/unlikeness User A experiences under different categories of environments, and User A's sensation/feelings/and self-awareness related to User A's whole body or specific body part as User A experiences the environment, also as User A inputs 162.

TABLE 3
Natural Environment (Example Location Category List)

| | | |
|---|---|---|
| Mountain | Frost | Grassland |
| Beach | Desert | River |
| Ocean | Lake | Sky |
| Underwater | Cave | Swamp |

Natural Environment (Example Weather and Season List)

| | | |
|---|---|---|
| Windy | Snow | Rain |
| Storm | Sunny | Cloudy |
| Winter | Spring | Summer |
| Fall | . . . | |

Further, space record logic 1206 may classify the outdoor human-made environment via the following dimensions: Road (Highway, local roads without lots of building nearby, local roads with lots of building nearby), Non-Road/Infrastructure (Size of human-made infrastructure: Huge, medium, small). Additionally, space record logic 1206 may classify the indoor human-made environment via the following two dimensions: level of brightness in the environment and size of the environment (e.g., room or hallway etc) Similar to the interface above described above, a graphic UI interface may be presented to User A to prompt User A to input his/her sensation/SA and the degree of likeness/unlikeness User A feels under different categories of environments as User A inputs 162.

Space record logic 1206 may classify the social environment via the following dimensions: Number of people in the environment based on computer vision (N), among these people how many of them are looking at the system and their facial expressions (e.g., positive or negative) based on facial recognition in computer vision (N_{Look}, N_{Positive}, N_{negative},), among these people how many of them are known friends/relatives/family members. Similar to the interface above described above, a graphic UI interface may be presented to User A to prompt User A to input his/her sensation/SA and the degree of likeness/unlikeness User A feels under different categories of environments as User A inputs 162.

Space record logic 1206 may classify general sensation/awareness related to familiar persons (friends/relatives/family members), based on the User A's feeling/awareness towards a specific familiar person, User A's degree of likeness/dislikeness towards that person, and the body party related to User A's feelings/awareness towards that person. Similar to the interface above described above, a graphic UI interface may be presented to User A to prompt User A to input his/her sensation/SA and the degree of likeness/unlikeness User A feels under different categories of environments as User A inputs 162.

All of the sensation and awareness information recorded by Space record logic 1206 under different natural, human-made, social environments for a specific human may be recorded to User A's personal database as a result of recording. On one embodiment, the personal database may be called database of recording component (DRC).

Space analysis logic 1208 may generally use computer vision to recognize visual objects and text in the three environments above described above. Further, space analysis logic 1208 uses speech recognition to recognize text from speech in the environments. In one embodiment, speech recognition may be implemented by voice to text logic 1308 of FIG. 13. Additionally, space analysis logic 1208 may use conceptnet.io/to obtain the sensation and descriptions of these objects and text in general.

Space generation logic 1210 generally relates generating a sensation and feeling for the digital copy of a user (for example, User A). This sensation and feeling for the digital copy of User A may be specific to the personal sensation and awareness of User A. Space generation logic 1210 may generate the sensation and feeling of “where I am” and “who I am with”.

Space generation logic 1210 may receive inputs from video/audio input 1212, clock/time input 1214, and GPS input 1216. For example, video/audio input 1212 send video and audio signals of a surrounding environment to space generation logic 1210. Similarly, clock/time input 1214 may send signals related to the local time of the surrounding environment to space generation logic 1210. GPS input 1216 may send signals related to the location of personal information collection system 1304 of FIG. 13. Space generation logic 1210 may receive the foregoing signals via information classification logic. Information classification logic may classify the signals received from video/audio input 1212, clock/time input 1214, and GPS input 1216 and store the information provided in such signals in a database of recording component (DRC). Space generation logic 1210 may then generate an individual specific sensation and awareness of the digital copy of User A based on the information stored in the DRC. For example, the individual specific sensation and awareness generated by space generation logic 1210 may cause the digital copy of User A to have self-awareness of who the digital copy of User A is, the type of environment that the digital copy of User A (or for example, the personal information collection system 1304 of FIG. 13) is in, the location of the digital copy of User A (or for example, the personal information collection system 1304 of FIG. 13), and who the digital copy of User A (or for example, the personal information collection system 1304 of FIG. 13) is with.

Space generation logic 1210 may also receive information from body dimension logic 168. Based on the information received from body dimension logic 168, space generation logic 1210 may cause the digital copy of User A to have self-awareness of what the digital copy of User A is doing. Additionally, space generation logic 1210 may generate a general sensation and self-awareness for the digital copy of user A.

In addition, the self-awareness information generated by space dimension generation logic 1210 may be integrated the output from body dimension logic 168. Table 4 below describes one embodiment of how the outputs generated by body dimension logic 168 may be integrated by space dimension generation logic 1210.

| Location (Based on | | |
|---|---|---|
| GPS input 1216 and | | |
| Video/audio input | Body Sensation (From body | Body Status “What I am doing” |
| 1212) | dimension logic 168) | (Basic classifications) |
| Natural Environment | Body Movement Speed | Walking; riding a biking (if my |
| | (Based on GPS): <5 KM per | legs are moving on a bike), running, |
| | hour; 5-30 KM per hour 30- | or on a slow car; car; train; high |
| | 150 KM per hour; >150 | speed train or airplane; |
| | KM/hour | |
| Human-made | Body Movement Speed | Working/studying/shopping/talking/ |
| environment (Inside or | (Based on GPS). Speed = 0: | sleeping depending on body |
| near a building) | Stationary; Speed <5 KM per | sensation; Walking; Running |
| | hour; Speed >10 KM per hour | |

Integrated dimension logic 174, as shown in FIG. 12, generally relates to integrating the information generated by time dimension logic 164, body dimension logic 168, and space dimension logic 172 to simulate an individual human's self-awareness for the digital copy of user A. To simulate an individual human's self-awareness for the digital copy of user A, integrated dimension logic 174 may first integrate all of sensations/feelings (for example, in text format) generated by time dimension logic 164, body dimension logic 168, and space dimension logic 172 into a textbox in a graphic interface of digital life system 100. For example, the text box may include body sensations/feelings (“what I feel”) related to body parts, time/date, self-awareness of space (“where I am” and “who is with me”), and “what I am doing” (see the figure below). Thus integrated dimension logic 174 generating the feeling of existence for the digital copy of User A which is close to the concept of a “soul”.

Second, digital life system 100 may be implemented in two versions. The full version of digital life system 100 may be implemented in a robot with sensors and one or more video cameras. The robot may be equipment with sensors as described in this disclosure. The smartphone version digital life system 100 may be composed of: 1) A smartphone, 2) a 3D hologram platform or any graphical representation of the person, and 3) a display for the hologram (e.g., IPad or anther smartphone or TV). All or portions of each of the personal information and memory collection logic 110, answer analysis logic 120, personal question and answer logic 130, active talking logic 150, human self-awareness and consciousness logic 160, and supporting functions logic 180 may be installed in the smartphone as an APP. The smartphone's GPS, light sensors, motion sensors etc. may serve as input devices of digital life system 100. The APP may be connected with a 3D hologram of human body via USB or Bluetooth. The 3D hologram platform may be implemented using commercially available 3D hologram platforms (e.g., Holographic Projection Pyramid available on Amazon).

For both full and smartphone versions, there may be an integrated view of the human self-awareness including a visual display showing a first-person view, an inner speech textbox of the all of sensations/feeling (text format) from time dimension logic 164, body dimension logic 168, and space dimension logic 172, and a 3D hologram of the human (or any graphical representation of the human) with graphical visualization of the feeling/sensation of the human (e.g., Red areas may indicate the feelings/sensation; Blue areas may indicate positive feeling/sensation).

The inner speech textbox may be visible or not visible to the user of digital life system 100 (for example, User B) depending on the privacy setting of the human whose self-awareness is being generated.

During the usage of digital life system 100 by a human (for example, User A or User B), the human may re-record or add new body part, time, or feelings towards different environments.

In some embodiments, third-party elements may be incorporated into digital life system 100. For example, computer vision may be used to recognize human face and their eyes. Similarly, face and voice recognition, smartphones, and commercially available 3D hologram platforms (for example, the 3D hologram platform available at https://www.amazon.com/Universal-Holographic-Projection-Pyramid-MediaPad/dp/B00UJRPB9Y/ref=sr_1_3?s=electronics&ie=UTF8&qid=1521920062&sr=1-3&keywords=3D+Hologram+Projector+for+tablet1) may be used.

The generated self-awareness of the digital copy of User A caused by human self-awareness and consciousness logic 160 may be verified against human User A's self-awareness under different situations. For example, in one embodiment, 12 scenarios (4 scenarios in different natural environments, 4 in human-made environments, and 4 in social environments) and 4 different times of day may be sampled to ask the 10 different humans to verbally report their subjective feelings/sensations in those different times and spaces. The same 10 humans may also use a graph of a human model to report their subjective feelings/sensations. Digital life system 100 may will also be used generate feelings/sensations in the same 12 scenarios. Pearson correlation (R) and root mean square (RMS) statistics may be used to measure the degree of match between the real human feelings/sensations and the feelings/sensations generated by digital life system 100.

FIG. 13 illustrates an example functional block diagram of a digital life system 100 comprising personal information collection system 1304 cloud-based storage and processing system 1300. In this embodiment, Personal information and collection system 1304 may be a user device such as a smartphone, table, personal computer, laptop, etc. Personal information and collection system 1304 may comprise User A inputs 112, User A inputs 162, sensor inputs 166, space inputs 170, display/audio logic 1306, voice to text logic 1308, and question list logic 144. Thus, personal information and collection system 1304 may allow a user to interact with digital life system 100 as described in the embodiments explained above. Personal information collection system 1304 may further communicate with cloud-based storage and processing system 1300 via network 1302.

Cloud-based storage and processing system 1300 may comprise components of personal information and memory collection logic 110 such as User A data tables 116, User A voice/video, experience logic 122, User A analysis logic 124, relationship logic 126. Further, cloud-based storage and processing system 1300 personal question and answer logic 130, active talking log 150, supporting functions logic 180, and components of human self-awareness and consciousness logic 160 such at time dimension logic 164, body dimension logic 168, space dimension logic 172 and integrated dimension logic 174. Thus cloud-based storage and processing system 1300 may comprise components of digital life system to support a user's interaction with the personal information collection system 1304 as described in the embodiments explained above by storing information and executing supporting processes.

Consistent with several embodiments of the present disclosure, digital life system 100 collects information from a user, for example User A, and simulates a digital copy of the user. Digital life system 100 further allows other users, for example User B, to interact with the simulated digital copy. According to the embodiment shown in FIG. 1, digital life system 100 includes personal information and memory collection logic 110, answer analysis logic 120, personal question and answer logic 130, active talking logic 150, human self-awareness and consciousness logic 160, and supporting functions logic 180.

As used in any embodiment herein, the term “logic” may refer to an application, software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices.

“Circuitry,” as used in any embodiment herein, may comprise, for example, singly or in any combination, hardwired circuitry, programmable circuitry, state machine circuitry, logic and/or firmware that stores instructions executed by programmable circuitry. The circuitry may be embodied as an integrated circuit, such as an integrated circuit chip. In some embodiments, the circuitry may be formed, at least in part, by the processors 108 executing code and/or instructions sets (e.g., software, firmware, etc.) corresponding to the functionality described herein, thus transforming a general-purpose processor into a specific-purpose processing environment to perform one or more of the operations described herein. In some embodiments, the various components and circuitry of the memory controller circuitry or other systems may be combined in a system-on-a-chip (SoC) architecture.

Embodiments of the operations described herein may be implemented in a computer-readable storage device having stored thereon instructions that when executed by one or more processors perform the methods. The processor may include, for example, a processing unit and/or programmable circuitry. The storage device may include a machine readable storage device including any type of tangible, non-transitory storage device, for example, any type of disk including floppy disks, optical disks, compact disk read-only memories (CD-ROMs), compact disk rewritables (CD-RWs), and magneto-optical disks, semiconductor devices such as read-only memories (ROMs), random access memories (RAMs) such as dynamic and static RAMs, erasable programmable read-only memories (EPROMs), electrically erasable programmable read-only memories (EEPROMs), flash memories, magnetic or optical cards, or any type of storage devices suitable for storing electronic instructions.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

What is claimed:

1. A digital life system comprising:
personal information and memory collection logic to receive personal information and personal memory data from a first user, the personal information and memory collection logic to generate data tables based on the personal information and personal memory data;
personal question and answer logic to generate responses to questions and statements submitted by a second user based on the data tables generated by the personal information and memory collection logic; and
active talking logic to generate questions and statements directed towards the second user, without first receiving a question or statement from the second user, based on the data tables created by the personal information and memory collection logic.

2. The digital life system of claim 1 wherein the data tables generated by the personal information and memory collection logic comprise a plurality of categories.

3. The digital life system of claim 2 wherein the personal question and answer logic further comprises:
question and answer preprocessing logic to extract pronouns from questions and statements from the second user, identify table-specific activation terms associated with one or more of the plurality of categories of the data tables, and determine a type of question submitted by the second user.

4. The digital life system of claim 3 wherein the personal question and answer logic further comprises:
table specific search logic to generate responses to questions and statements submitted by a second user based on the table-specific activation terms identified by the question and answer preprocessing logic.

5. The digital life system of claim 4 wherein the personal question and answer logic further comprises:
pronoun/keyword search logic to generate responses to questions and statements submitted by a second user based on pronouns extracted by the question and answer preprocessing logic.

6. The digital life system of claim 5 wherein the personal question and answer logic further comprises:
extraction/keyword logic to activate the table specific search logic and/or the pronoun/keyword search logic based on the pronouns extracted by the question and answer preprocessing logic and the table-specific activation terms identified by the question and answer preprocessing logic.

7. The digital life system of claim 6 wherein the personal question and answer logic further comprises:
consequence logic to generate responses based on a reply submitted by the second user in response to responses generated by the personal question and answer logic and the table specific search logic.

8. The digital life system of claim 7 wherein the personal question and answer logic further comprises:
extraction/keyword logic to activate the table specific search logic and/or the pronoun/keyword search logic based on the pronouns extracted by the question and answer preprocessing logic and the table-specific activation terms identified by the question and answer preprocessing logic.

9. The digital life system of claim 8 wherein the table specific search logic performs an upper-level search for table-specific activation terms when a match to the table-specific activation term is not identified, and further performs a lower-level search for table-specific activation terms when the upper-level search fails to identify table-specific activation terms.

10. The digital life system of claim 9 wherein the extraction/keyword logic is to further:
activate the consequence logic based on the question type determined by the question and answer preprocessing logic.

11. The digital life system of claim 10 wherein the active talking logic further comprises:
active talking preprocessing logic to;
determine a closeness of relationship between the first user and the second user;
determine whether the relationship between the first user and the second user is positive or negative; and
determine a degree of selfishness for the first user.

12. The digital life system of claim 11 wherein the active talking logic further comprises:
active dialogue logic to generate the questions and statements directed towards the second user based on the closeness of relationship between the first user and the second user, whether the relationship between the first user and the second user is positive or negative, and the degree of selfishness of the first user.

13. The digital life system of claim 12 wherein the active dialogue logic is to further generate different types of questions and statements directed towards the second user based on predefined probabilities.

14. The digital life system of claim 13 wherein the active dialogue logic is to further determine whether a reply submitted by the second user, in response to the questions and statements responses generated by the active dialogue logic, is positive or negative, and generate additional questions and statements directed towards the second user based whether the reply is positive or negative.

15. The digital life system of claim 14 where the active talking logic further comprises:
time management logic to:
determine a start time of dialogue between a digital copy of the first user and the second user;
determine a current time for the second user;
determine whether the current time for the second user is within a comfortable time window of the digital copy of the first user; and
determine a total time of dialogue between a digital copy of the first user and the second user based on the start time of dialogue and the current time for the second user.

16. The digital life system of claim 15 where the time management logic to further:
terminate operations of the active talking logic based on whether the current time for the second user is within a comfortable time window of the digital copy of the first user and based on the total time of dialogue between a digital copy of the first user and the second user.

17. The digital life system of claim 16 where the time management logic to further:
determine whether the first user is an extrovert type person based on the data tables;
determine a time of silence between responses of the second user; and
activate the active dialogue logic based on the time of silence between responses and based on whether the first user is an extrovert type person.

18. The digital life system of claim 17 wherein the active talking logic further comprises:
knowledge update logic to:
determine whether a reply submitted by the second user, in response to the questions and statements responses generated by the active dialogue logic, is positive or negative;
determine a total number of negative replies submitted by the second user;
modify the relationship between the second user and the digital copy of the first user based on the total number of negative replies submitted by the second user.

19. The digital life system of claim 1 further comprising:
human self-awareness and consciousness logic comprising
time dimension logic to receive biological clock information from the first user, the time dimension logic to cause the generation of verbal and visual expressions associated with the digital copy of the first user based on the biological clock information;
body dimension logic to receive body dimension information from the first user, the body dimension logic to cause the generation of verbal and visual expressions associated with the digital copy of the first user based on the body dimension information; and
space dimension logic to receive space dimension information from the first user, the space dimension logic to cause the generation of verbal and visual expressions associated with the digital copy of the first user based on the space dimension information.

20. The digital life system of claim 19 wherein the human self-awareness and consciousness logic further comprises:
integrated dimension logic to receive information from the time dimension logic, body dimension logic, and space dimension logic, the integrated dimension logic to cause the generation of integrated verbal and visual expressions associated with the digital copy of the first user based on the information from the time dimension logic, body dimension logic, and space dimension logic.
